(19) United States
(12) Patent Application Publication
(10) Pub. No.: US 2006/0167106 A1
(43) Pub. Date: Jul. 27, 2006

(54) Compounds acting at the centrosome

(76) Inventors: Mei Zhang, Lexington, MA (US); Andras Ladanyi, Budapest (HU)

Correspondence Address: HAMILTON, BROOK, SMITH & REYNOLDS, P.C., 530 VIRGINIA ROAD, P.O. BOX 9133, CONCORD, MA, 01742-9133, US

(21) Appl. No.: 11/283,570

(22) Filed: Nov. 18, 2005

Related U.S. Application Data
(60) Provisional application No. 60/629,858, filed on Nov. 19, 2004.

(51) Int. Cl. A61K 31/16; C07C 327/40

(52) U.S. Cl. 514/599; 564/74; 564/76

(57) ABSTRACT

The present invention relates to compounds, and methods utilizing compounds, which exhibit one or more of the following properties: i) disrupts organization of an actin cytoskeleton of a cell; ii) disrupts organization of a microtubule network of a cell; iii) induces accumulation of tubulin at centrosomes but does not induce accumulation of tubulin in a nucleus of a cell; iv) induces accumulation of tubulin at centrosomes at a concentration of 500 nM or less within four hours; v) induces accumulation of Hsp70 and has weak-to-moderate proteasome inhibitory activity; and vi) does not have proteasome inhibitory activity when assayed on purified proteasomes.

### RELATED APPLICATIONS

This application claims the benefit of U.S. Provisional Application No. 60/629,858, filed Nov. 19, 2004, which is incorporated by reference herein in its entirety.

### FIELD OF THE INVENTION

The invention relates to certain bis(thio-hydrazide amide) compounds affecting activity at the centrosome of the cell and their use in treating diseases.

### BACKGROUND OF THE INVENTION

The centrosome of the cell is responsible for nucleating and organizing microtubules. Microtubules (which are composed of the protein tubulin) and other polymers, such as actin filaments, make up the cytoskeleton. The cytoskeleton is involved in cell shape, structure, movement and cellular division, and thus disruption of the organization of the cytoskeleton can affect many important biological processes. For instance, microtubule assembly and disassembly is necessary for mitosis, and inhibition of either the assembly or disassembly of microtubules interferes with cell proliferation. Rapid or abnormal cell proliferation is linked to many diseases, such as cancer.

Additionally, proteasome complexes are localized to the centrosomes and are present at a number of other cellular locations, where they are involved in protein degradation (Wigley et al., J. Cell Biol. 145:481-490 (1999); the entire teachings of which are incorporated herein by reference). Inhibition of proteasome activity results in the accumulation of proteins (e.g., tubulin) that are subject to proteasome degradation.

Decreasing the activity of the ubiquitin-proteasome system has shown promise as a treatment for cancerous and non-cancerous proliferative disorders, cystic fibrosis, and conditions marked by excessive or accelerated protein degradation, such as muscle-wasting diseases and skeletal system disorders. Heat shock proteins (Hsp's) are a group of proteins that are induced in response to cellular stress. Increased expression of proteins in the Hsp 70 family are known to protect a broad range of cells under stress by inhibiting various cellular death pathways, such as apoptosis (Mosser, et al., Mol. Cell Biol., 2000 October; 20(19): 7146-7159; Yenari, Adv. Exp. Med. Biol., 2002, 513, 281-299; Kiang and Tsokos, Pharmacol. Ther., 1998; 80(2):182-201). For example, it is known in the art that a variety of medical conditions can experience a protective effect in response to Hsp70.

Given the potential of compounds that affect centrosome activity to treat and/or alleviate a variety of disease pathologies, it is desirable to identify additional agents that act at the centrosome. Furthermore, it is also desirable to identify novel compounds acting at the centrosome that display increased efficacy and/or possess other advantageous properties for treating particular diseases (e.g., decreased toxicity).

### SUMMARY OF THE INVENTION

In one embodiment, the invention is a compound that exhibits one or more of a subset of properties. The compounds are able to: i) disrupt organization of an actin cytoskeleton of a cell; ii) disrupt organization of a microtubule network of a cell; iii) induce accumulation of tubulin at centrosomes but not induce accumulation of tubulin in a nucleus of a cell; iv) induce accumulation of tubulin at centrosomes at a concentration of 500 nM or less within four hours; v) induce accumulation of Hsp70 but only possess weak-to-moderate proteasome inhibitory activity; and/or vi) not possess proteasome inhibitory activity when assayed on purified proteasomes. In this embodiment, the compound is not a bis(thio-hydrazide amide) represented by Structural Formula (I):

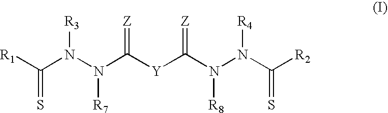

wherein Y is a covalent bond or a substituted or unsubstituted straight chained hydrocarbyl group, or, Y, taken together with both >C═Z groups to which it is bonded, is a substituted or unsubstituted aromatic group;

R₁-R₄ are independently —H, an aliphatic group, a substituted aliphatic group, an aryl group or a substituted aryl group, or R₁ and R₃ taken together with the carbon and nitrogen atoms to which they are bonded, and/or R₂ and R₄ taken together with the carbon and nitrogen atoms to which they are bonded, form a non-aromatic heterocyclic ring optionally fused to an aromatic ring;

R₅ and R₆ are each independently —H, an aliphatic or substituted aliphatic group, or R₅ is —H and R₆ is a substituted or unsubstituted aryl group, or, R₅ and R₆, taken together, are a C2-C6 substituted or unsubstituted alkylene group;

R₇-R₈ are independently —H, an aliphatic group, a substituted aliphatic group, an aryl group or a substituted aryl group. Preferably, R₇ and R₈ are the same; and

Z is ═O or ═S.

In another embodiment, the invention is a method of disrupting centrosome activity in a subject in need thereof comprising administering an effective amount of a compound of the invention. In a particular embodiment, the subject in need thereof has a condition selected from the group consisting of a cancer, a non-cancerous proliferative condition and an Hsp70-responsive disorder.

In another embodiment, the invention is a method for treating a condition in a subject comprising administering an effective amount of a compound that exhibits one or more of a subset of properties. The compounds are able to: i) disrupt organization of an actin cytoskeleton of a cell; ii) disrupt organization of a microtubule network of a cell; iii) induce accumulation of tubulin at centrosomes but not induce accumulation of tubulin in a nucleus of a cell; iv) induce accumulation of tubulin at centrosomes at a concentration of 500 nM or less within four hours; v) induce accumulation of Hsp70 but only possess weak-to-moderate proteasome inhibitory activity; and/or vi) not possess proteasome inhibitory activity when assayed on purified proteasomes. In this embodiment, suitable conditions for treatment include muscle-wasting diseases (e.g., fever, muscle disuse (atrophy) and denervation, nerve injury, fasting, renal failure associated with acidosis, hepatic failure, uremia, diabetes, and sepsis), skeletal system disorders resulting from bone loss or low bone density (e.g., closed fractures, open fractures, non-union fractures, age-related osteoporosis, post-menopausal osteoporosis, glucocorticoid-induced osteoporosis, disuse osteoporosis, arthritis), growth deficiencies (e.g., periodontal disease and defects, cartilage defects or disorders, disorders of hair growth (e.g., male pattern baldness, alopecia caused by chemotherapy, hair thinning resulting from aging, genetic disorders resulting in deficiency of hair coverage)), dry-eye disorders (e.g., excessive inflammation in relevant ocular tissues, such as the lacrimal and meibomian glands, dry eye associated with refractive surgery (e.g., LASIK surgery) and cystic fibrosis. In a particular embodiment, the compound is a compound represented by Structural Formula (I). In another embodiment, the compound is a compound represented by the following structural formula: 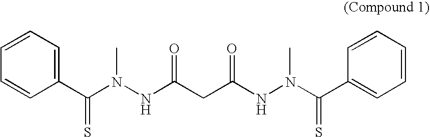
or a pharmaceutically-acceptable salt thereof.

In other embodiments, the invention is a method of identifying a compound that induces accumulation of proteins at centrosomes, but does not induce accumulation of proteins in the nucleus of a cell. In one embodiment, the method comprises combining a cell that expresses tubulin and a test agent, and measuring the accumulation of tubulin at one or more centrosomes and/or in the nucleus of the cell. In this embodiment, an increase in the accumulation of tubulin at the centrosome(s), but no increase in the accumulation of tubulin at the nucleus, relative to a suitable control, indicates that the test agent is a compound that induces accumulation of tubulin at centrosomes but does not induce accumulation of tubulin in the nucleus of a cell.

In other embodiments, the invention is a method for identifying a compound that disrupts centrosome activity comprising combining a cell that expresses a centrosome-associated protein and a test agent; and measuring the accumulation of the centrosome-associated protein at one or more centrosomes of the cell and in a nucleus of the cell. An increase in the accumulation of the centrosome-associated protein at the one or more centrosomes, but no increase in the accumulation of the centrosome-associated protein at the nucleus, relative to a suitable control, indicates that said test agent is a compound that disrupts centrosome activity.

In other embodiments, the invention is a method of identifying a proteasome inhibitor. In one embodiment, the method comprises combining a cell that expresses tubulin and a test agent, and measuring the accumulation of tubulin at one or more centrosomes and/or in the nucleus of the cell. In this embodiment, an increase in the accumulation of tubulin at the centrosome(s) and/or in the nucleus, relative to a suitable control, indicates that the test agent is a proteasome inhibitor.

In another embodiment, the invention is a method of identifying a centrosomal proteasome inhibitor comprising combining a cell that expresses tubulin and a test agent, and measuring the accumulation of tubulin at one or more centrosomes of the cell and in the nucleus of the cell. In this embodiment, an increase in the accumulation of tubulin at the centrosome(s), but no increase in the accumulation of tubulin in the nucleus, relative to a suitable control, indicates that the test agent is a centrosomal proteasome inhibitor.

In another embodiment, the invention is a method of identifying a proteasome inhibitor comprising combining a cell that expresses a centrosome-associated protein and a test agent, and measuring the accumulation of the centrosome-associated protein at one or more centrosomes of the cell. In this embodiment, an increase in the accumulation of the centrosome-associated protein at the centrosome(s), relative to a suitable control, indicates that the test agent is a proteasome inhibitor.

Suitable centrosome-associated proteins that can be used in embodiments of this invention, include, but are not limited to, pericentrin, CP140, centrin, alpha-tubulin, beta-tubulin, gamma-tubulin, AKAP450, SKP1p, cyclin-dependent kinase 2-cyclin E (Cdk2-E), kendrin, Protein kinase C-theta, EB1 protein, Nek2, protein kinase A type II isozymes, Hsp70, heat shock Cognate 70 (HSC70), PH33, AIKs, human SCF(SKP2) subunit p19(SKP1), STK15/BTAK, C-Nap1, Tau-like proteins, cyclin E, p53, retinoblastoma protein pRB, BRCA1, dynein and NuMA.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIG. 1A depicts a fluorescent image showing localization of α-tubulin-YFP α-tubulin protein tagged with Yellow Fluorescent Protein (YFP)) in Chinese hamster ovary (CHO) cells that have been treated for 5 hours with dimethylsulfoxide (DMSO).

FIG. 1B depicts a fluorescent image of α-tubulin-YFP localization in CHO cells treated for 5 hours with 10 nM Taxol.

FIG. 1C depicts effects of Compound 1 on centrosome structure. A fluorescent image shows α-tubulin-YFP localization in CHO cells treated for 5 hours with 0.5 μM of Compound 1.

FIG. 1D depicts effects of Compound 1 and Taxol on centrosome structure. A fluorescent image shows α-tubulin-YFP localization in CHO cells treated for 5 hours with a combination of Compound 1 (0.5 μM) and Taxol (10 nM). The image shows accumulation of α-tubulin-YFP at the centrosomes.

FIG. 2A depicts a fluorescent image showing α-tubulin-YFP localization in CHO cells treated for 11 hours with DMSO.

FIG. 2B depicts a fluorescent image showing α-tubulin-YFP localization in CHO cells treated for 11 hours with Taxol (10 nM).

FIG. 2C depicts effects of Compound 1 on centrosome structure. A fluorescent image shows α-tubulin-YFP localization in CHO cells treated for 11 hours with Compound 1 (0.5 μM). The image shows accumulation of α-tubulin-YFP at the centrosomes.

FIG. 2D depicts effects of Compound 1 and Taxol on centrosome structure. A fluorescent image shows α-tubulin-YFP localization in CHO cells treated for 11 hours with a combination of Compound 1 (0.5 μM) and Taxol (10 nM). The image shows accumulation of α-tubulin-YFP at the centrosomes.

FIG. 3A depicts effects of Compound 1 and Taxol on centrosome structure. A fluorescent image shows nuclei stained with 4′,6-Diamidino-2-phenylindole (DAPI) in CHO cells that have been treated with Taxol (10 nM) plus Compound 1 (0.5 μM) for 5 hours.

FIG. 3B depicts effects of Compound 1 and Taxol on centrosome structure. A fluorescent image shows gamma-tubulin (γ-tubulin) staining in CHO cells treated with Taxol (10 nM) plus Compound 1 (0.5 μM) for 5 hours.

FIG. 3C depicts effects of Compound 1 and Taxol on centrosome structure. A fluorescent image shows alpha-tubulin (α-tubulin)-YFP localization in CHO cells treated with Taxol (10 nM) plus Compound 1 (0.5 μM) for 5 hours.

FIG. 3D depicts the merged image of FIGS. 3B and 3C. The image shows colocalization of α-tubulin-YFP with γ-tubulin at the centrosomes.

FIG. 4A depicts a fluorescent image showing localization of α-tubulin in CV-1 (Normal African Green Monkey Kidney Fibroblast) cells treated with 0.5 μM Taxol for 5 hours.

FIG. 4B depicts a fluorescent image showing localization of a centrosomal protein, pericentrin, in CV-1 cells treated with 0.5 μM Taxol for 5 hours.

FIG. 4C depicts the merged image of FIGS. 4A and 4B and includes DAPI-stained nuclei.

FIG. 4D depicts effects of Compound 1 on centrosome structure. A fluorescent image shows localization of α-tubulin in CV-1 cells treated with 0.5 μM Compound 1 for 5 hours.

FIG. 4E depicts effects of Compound 1 on centrosome structure. A fluorescent image shows localization of a centrosomal protein, pericentrin, in CV-1 cells treated with 0.5 μM Compound 1 for 5 hours. The image shows accumulation of pericentrin at the center of cells.

FIG. 4F depicts the merged image of FIGS. 4D and 4E and includes DAPI-stained nuclei.

FIG. 5A depicts a fluorescent image showing α-tubulin-YFP localization in CHO cells treated for 5 hours with DMSO as a control.

FIG. 5B depicts fluorescent images showing the accumulation of α-tubulin-YFP at the centrosomes and perinuclear regions in CHO cells treated for 5 hours with 0.5 μM Compound 1. The arrows indicate accumulation of α-tubulin-YFP at the centrosomes.

FIG. 5C depicts a fluorescent image showing the accumulation of α-tubulin-YFP at the centrosomes and perinuclear regions in CHO cells treated for 5 hours with 10 μM MG132. The arrows indicate accumulation of α-tubulin-YFP at the centrosomes.

FIG. 5D depicts a fluorescent image showing the accumulation of α-tubulin-YFP at the centrosomes and perinuclear regions in CHO cells treated for 5 hours with 100 μM ALLN. The arrows indicate accumulation of α-tubulin-YFP at the centrosomes.

FIG. 5E depicts a fluorescent image showing the accumulation of α-tubulin-YFP at the centrosomes and perinuclear regions in CHO cells treated for 5 hours with 10 μM Lactacystin. The arrows indicate accumulation of α-tubulin-YFP at the centrosomes.

FIG. 6A depicts a fluorescent image showing α-tubulin-YFP localization in CHO cells treated for 24 hours with 0.1 μM Taxol. The image shows lack of accumulation of tubulin-YFP at the centrosomes.

FIG. 6B depicts a fluorescent image showing α-tubulin-YFP localization in CHO cells treated for 24 hours with 10 μM Epothilone D. The image shows lack of accumulation of tubulin-YFP at the centrosomes.

FIG. 6C depicts a fluorescent image showing α-tubulin-YFP localization in CHO cells treated for 24 hours with 1 μM Vincristine. The image shows lack of accumulation of tubulin-YFP at the centrosomes.

FIG. 6D depicts a fluorescent image showing α-tubulin-YFP localization in CHO cells treated for 24 hours with 10 μM Compound 4. The image shows lack of accumulation of tubulin-YFP at the centrosomes.

FIG. 6E depicts a fluorescent image showing α-tubulin-YFP localization in CHO cells treated for 24 hours with 0.5 μM Compound 1. The image shows accumulation of tubulin-YFP at the centrosomes.

FIG. 6F depicts a fluorescent image showing α-tubulin-YFP localization in CHO cells treated for 24 hours with 100 μM ALLN. The image shows accumulation of tubulin-YFP at the centrosomes.

FIG. 6G depicts a fluorescent image showing α-tubulin-YFP localization in CHO cells treated for 24 hours with 10 μM Lactacystin. The image shows accumulation of tubulin-YFP at the centrosomes.

FIG. 6H depicts a fluorescent image showing α-tubulin-YFP localization in CHO cells treated for 24 hours with 10 μM MG132. The image shows accumulation of tubulin-YFP at the centrosomes.

FIG. 7A depicts a fluorescent image of α-tubulin-YFP localization in CHO cells that have been treated for 4 hours with 100 nM Compound 1. The arrows show accumulation of α-tubulin-YFP at centrosomes. The image shows accumulation of tubulin-YFP at the centrosomes (red arrows).

FIG. 7B depicts a fluorescent image of α-tubulin-YFP localization in CHO cells that have been treated for 4 hours with 500 nM Compound 1. The arrows show accumulation of α-tubulin-YFP at centrosomes. The image shows accumulation of tubulin-YFP at the centrosomes (red arrows).

FIG. 7C depicts a fluorescent image of α-tubulin-YFP localization in CHO cells that have been treated for 4 hours with 500 nM Drug-V (Velcade). The image shows lack of accumulation of tubulin-YFP at the centrosomes.

FIG. 7D depicts a fluorescent image of α-tubulin-YFP localization in CHO cells that have been treated for 4 hours with 5 μM Compound 3. The image shows lack of accumulation of tubulin-YFP at the centrosomes.

FIG. 8 is a graph depicting the effects of GMP-grade Compound 1 on proteasome activity in vitro. Activity was monitored for 120 minutes. Compound 1 concentrations of 25 μM and 50 μM were tested. Controls included DMSO (1:1), no enzyme, and enzyme without the drug samples (labeled “Enzyme”).

FIG. 9 is a graph depicting the effect of GMP-grade Compound 2 (the salt form of Compound 1) on proteasome activity in vitro. Activity was monitored for 120 minutes. Compound 2 concentrations of 25 μM and 50 μM were tested. Controls included mannitol, no enzyme, and enzyme without the drug samples (labeled “Enzyme”). Velcade (0.5 μM) was used as a positive control for proteasome inhibition.

FIG. 10 is a graph depicting the effect of the proteasome inhibitor Velcade on proteasome activity using an in vitro assay. Activity was monitored for 120 minutes. Concentrations of Velcade between 5 nM and 50 μM were tested. Controls included DMSO, Normal saline, and Mannitol (each at 1:1), as well as no enzyme and enzyme without the drug controls (labeled “Enzyme”).

FIG. 11A depicts a fluorescent image using identical imaging settings showing localization of a Green Fluorescent Protein (GFP)-labeled proteasome-targeting chimera protein (proteasome-sensor protein) in HEK-293 cells treated with Compound 1 at a 1 nM concentration. The image shows accumulation of proteasome-sensor protein in cells.

FIG. 11B depicts a fluorescent image using identical imaging settings showing localization of a Green Fluorescent Protein (GFP)-labeled proteasome-targeting chimera protein (proteasome-sensor protein) in HEK-293 cells treated with Compound 1 at a 5 nM concentration. The image shows accumulation of proteasome-sensor protein in cells.

FIG. 11C depicts a fluorescent image using identical imaging settings showing localization of a Green Fluorescent Protein (GFP)-labeled proteasome-targeting chimera protein (proteasome-sensor protein) in HEK-293 cells treated with Compound 1 at a 10 nM concentration. The image shows accumulation of proteasome-sensor protein in cells.

FIG. 11D depicts a fluorescent image using identical imaging settings showing localization of a Green Fluorescent Protein (GFP)-labeled proteasome-targeting chimera protein (proteasome-sensor protein) in HEK-293 cells treated with Compound 1 at a 50 nM concentration. The image shows accumulation of proteasome-sensor protein in cells.

FIG. 11E depicts a fluorescent image using identical imaging settings showing localization of a Green Fluorescent Protein (GFP)-labeled proteasome-targeting chimera protein (proteasome-sensor protein) in HEK-293 cells treated with Compound 1 at a 100 nM concentration. The image shows accumulation of proteasome-sensor protein in cells.

FIG. 11F depicts a fluorescent image using identical imaging settings showing localization of a Green Fluorescent Protein (GFP)-labeled proteasome-targeting chimera protein (proteasome-sensor protein) in HEK-293 cells treated with Compound 1 at a 500 nM concentration. The image shows accumulation of proteasome-sensor protein in cells.

FIG. 11G depicts a fluorescent image using identical imaging settings showing localization of proteasome-sensor protein in HEK-293 cells treated with Drug-V (Velcade) at a 1 nM concentration for 20 hours. The image shows accumulation of proteasome-sensor protein in cells.

FIG. 11H depicts a fluorescent image using identical imaging settings showing localization of proteasome-sensor protein in HEK-293 cells treated with Drug-V (Velcade) at a 5 nM concentration for 20 hours. The image shows accumulation of proteasome-sensor protein in cells.

FIG. 11I depicts a fluorescent image using identical imaging settings showing localization of proteasome-sensor protein in HEK-293 cells treated with Drug-V (Velcade) at a 10 nM concentration for 20 hours. The image shows accumulation of proteasome-sensor protein in cells.

FIG. 11J depicts a fluorescent image using identical imaging settings showing localization of proteasome-sensor protein in HEK-293 cells treated with Drug-V (Velcade) at a 50 nM concentration for 20 hours. The image shows accumulation of proteasome-sensor protein in cells.

FIG. 11K depicts a fluorescent image using identical imaging settings showing localization of proteasome-sensor protein in HEK-293 cells treated with Drug-V (Velcade) at a 100 nM concentration for 20 hours. The image shows accumulation of proteasome-sensor protein in cells.

FIG. 11L depicts a fluorescent image using identical imaging settings showing localization of proteasome-sensor protein in HEK-293 cells treated with Drug-V (Velcade) at a 500 nM concentration for 20 hours. The image shows accumulation of proteasome-sensor protein in cells.

FIG. 12A depicts a fluorescent image showing localization of a GFP-labeled proteasome-targeting chimera protein in HEK-293 cells treated for 24 hours with DMSO. The image shows lack of proteasome-sensor protein in cells.

FIG. 12B depicts a fluorescent image showing localization of a GFP-labeled proteasome-targeting chimera protein in HEK-293 cells treated for 24 hours with 100 nM Taxol. The image shows lack of proteasome-sensor protein in cells.

FIG. 12C depicts a fluorescent image showing localization of a GFP-labeled proteasome-targeting chimera protein in HEK-293 cells treated for 24 hours with 0.5 μM Compound 1. The image shows accumulation of proteasome-sensor protein in cells.

FIG. 12D depicts a fluorescent image showing localization of a GFP-labeled proteasome-targeting chimera protein in HEK-293 cells treated for 24 hours with 0.5 μM Compound 2. The image shows accumulation of proteasome-sensor protein in cells.

FIG. 12E depicts a fluorescent image showing localization of a GFP-labeled proteasome-targeting chimera protein in HEK-293 cells treated for 24 hours with Taxol (100 nM) plus Compound 1 (0.5 μM). The image shows accumulation of proteasome-sensor protein in cells.

FIG. 12F depicts a fluorescent image showing localization of a GFP-labeled proteasome-targeting chimera protein in HEK-293 cells treated for 24 hours with 100 nM Velcade. The image shows accumulation of proteasome-sensor protein in cells.

FIG. 13A depicts non-gated data graphs showing the results of flow cytometry analysis performed on HEK-293 proteasome-sensor cells subjected to treatment with DMSO. The red arrow indicates a significant increase of fluorescence following 20 hours of treatment.

FIG. 13B depicts non-gated data graphs showing the results of flow cytometry analysis performed on HEK-293 proteasome-sensor cells subjected to treatment with 500 nM Compound 1. The red arrow indicates a significant increase of fluorescence following 20 hours of treatment.

FIG. 13C depicts non-gated data graphs showing the results of flow cytometry analysis performed on HEK-293 proteasome-sensor cells subjected to treatment with 100 nM Velcade. The red arrow indicates a significant increase of fluorescence following 20 hours of treatment.

FIG. 14A depicts a phase-contrast image of an HEK-293 cell colony expressing a GFP-labeled proteasome-targeting chimera protein after treatment with 500 nM Compound 1 for 20 hours. The red arrow indicates the periphery of the colony and the blue arrow indicates the center of the colony.

FIG. 14B depicts a fluorescent image of an HEK-293 cell colony expressing a GFP-labeled proteasome-targeting chimera protein, indicating stronger accumulation at the periphery of the colony (red arrow) relative to the center (blue arrow) after treatment with 500 nM Compound 1 for 20 hours.

FIG. 14C depicts a phase-contrast image of an HEK-293 cell colony expressing a GFP-labeled proteasome-targeting chimera protein after treatment with 500 nM Compound 1 for 40 hours. The red arrow indicates the periphery of the colony and the blue arrow indicates the center of the colony.

FIG. 14D depicts a fluorescent image of an HEK-293 cell colony expressing a GFP-labeled proteasome-targeting chimera protein, indicating stronger accumulation at the periphery of the colony (red arrow) relative to the center (blue arrow) after treatment with 500 nM Compound 1 for 40 hours.

FIG. 14E depicts a fluorescent image of an HEK-293 cell colony expressing a GFP-labeled proteasome-targeting chimera protein after treatment with 500 nM Velcade for 20 hours. The yellow arrow indicates cells in the center of colony.

FIG. 14F depicts a fluorescent image of an HEK-293 cell colony expressing a GFP-labeled proteasome-targeting chimera protein after treatment with 50 nM Velcade for 20 hours. The yellow arrow indicates cells in the center of colony.

FIG. 15 depicts a high-resolution image of an HEK-293 proteasome-sensor cell showing a general, broad distribution of GFP-labeled proteasome-targeting chimera protein in both the cytosol and nucleus following treatment with 5 μM Compound 1 for 24 hours.

FIG. 16A depicts a fluorescent image of α-tubulin localization in CV-1 cells that have been treated with DMSO.

FIG. 16B depicts a fluorescent image of α-tubulin localization in CV-1 cells that have been treated with DMSO.

FIG. 16C depicts a fluorescent image of α-tubulin localization in CV-1 cells that have been treated with DMSO.

FIG. 16D depicts a fluorescent image of α-tubulin localization indicating disruption of the microtubule network in CV-1 cells that have been treated with 0.5 μM Compound 1 for 5 hours.

FIG. 16E depicts fluorescent images of α-tubulin localization indicating disruption of the microtubule network in CV-1 cells that have been treated with 0.5 μM Compound 1 for 5 hours.

FIG. 16F depicts fluorescent images of α-tubulin localization indicating disruption of the microtubule network in CV-1 cells that have been treated with 0.5 μM Compound 1 for 5 hours.

FIG. 17A depicts an image of a-tubulin immunofluorescence showing the microtubule network in CV-1 cells that have been treated with 0.5 μM Compound 1 for 5 hours. DAPI staining of nuclei is included.

FIG. 17B depicts a higher magnification view of a cell in FIG. 17A and displays α-tubulin localization in the cytoplasm.

FIG. 17C depicts a higher magnification view of a cell in FIG. 17A and displays α-tubulin localization around the nuclei.

FIG. 17D depicts a higher magnification view of a cell in FIG. 17A and displays α-tubulin localization in the cytoplasm.

FIG. 17E depicts a higher magnification view of a cell in FIG. 17A and displays α-tubulin localization around the nuclei.

FIG. 18A depicts the microtubule network in CV-1 cells treated for 6 hours with DMSO using indirect immunofluorescence to detect α-tubulin.

FIG. 18B is a higher magnification image of cells shown in FIG. 18A.

FIG. 18C depicts the microtubule network in CV-1 cells treated for 6 hours with GMP-grade Compound 1 (0.5 μM) using indirect immunofluorescence to detect α-tubulin.

FIG. 18D is a higher magnification image of cells shown in FIG. 18C.

FIG. 18E depicts the microtubule network in CV-1 cells treated for 6 hours with Velcade (0.5 μM) using indirect immunofluorescence to detect α-tubulin.

FIG. 18F is a higher magnification image of cells shown in FIG. 18E.

FIG. 19A depicts a phase-contrast time-lapse image showing changes in the shape of live CV-1 cells after 0 hours of treatment with 500 nM Compound 1.

FIG. 19B depicts a phase-contrast time-lapse image showing changes in the shape of live CV-1 cells after 2 hours of treatment with 500 nM Compound 1.

FIG. 19C depicts a phase-contrast time-lapse image showing changes in the shape of live CV-1 cells after 4 hours of treatment with 500 nM Compound 1. The red arrows (pointing toward the center of the cell) indicate shrinkage of cell bodies and the yellow arrows indicate existence of focal adhesions.

FIG. 19D depicts a phase-contrast image of a CV-1 cell after 2 hours of treatment with 500 μM Compound 1.

FIG. 19E depicts a phase-contrast image of a CV-1 cell after 4 hours of treatment with 500 μM Compound 1. The red arrows indicate attachment of the cell membrane to the culture surface and the yellow arrows indicate shrinkage of cell body relative to FIG. 19D.

FIG. 19F depicts a fluorescent image of α-tubulin-YFP localization in the CV-1 cell shown in FIG. 19D after 2 hours of treatment with 500 μM Compound 1.

FIG. 19G depicts a fluorescent image of α-tubulin-YFP localization in the CV-1 cell shown in FIG. 19E after 4 hours of treatment with 500 μM Compound 1. The yellow arrows indicate shrinkage of the cell body relative to FIG. 19F.

FIG. 20A depicts a fluorescent image showing α-tubulin-YFP localization in MCF-7 cells 40 hours after treatment with DMSO for 25 hours.

FIG. 20B depicts a fluorescent image showing α-tubulin-YFP localization in MCF-7 cells 40 hours after treatment with 500 nM Compound 1 for 25 hours. The arrow indicates accumulation of α-tubulin-YFP at centrosome-like structures.

FIG. 20C depicts a fluorescent image showing α-tubulin-YFP localization in MCF-7 cells 40 hours after treatment with 100 nM Taxol for 25 hours.

FIG. 20D depicts a fluorescent image showing c-tubulin-YFP localization in MCF-7 cells 40 hours after treatment with a combination of Compound 1 (500 nM) plus Taxol (100 nM) for 25 hours. The arrow indicates accumulation of α-tubulin-YFP at centrosome-like structures.

FIG. 21A depicts a fluorescent image of α-tubulin immunofluorescence in a CV-1 cell treated with DMSO for 6 hours.

FIG. 21B depicts a fluorescent image of α-tubulin immunofluorescence in a CV-1 cell treated with 0.5 μM Compound 1 for 6 hours.

FIG. 22A depicts a fluorescent image of Alexa 488 conjugated Phalloidin, which binds to F-actin, in CV-1 cells that have been treated for 6 hours with DMSO.

FIG. 22B depicts a fluorescent image of Alexa 488 conjugated Phalloidin, which binds to F-actin, in CV-1 cells that have been treated for 6 hours with Taxol (100 nM).

FIG. 22C depicts a fluorescent image of Alexa 488 conjugated Phalloidin, which binds to F-actin, in CV-1 cells that have been treated for 6 hours with Compound 1 (0.5 μM).

FIG. 22D depicts a fluorescent image of Alexa 488 conjugated Phalloidin, which binds to F-actin, in CV-1 cells that have been treated for 6 hours with Taxol (100 nM) plus Compound 1 (0.5 μM).

FIG. 23A depicts a time-lapse phase-contrast image showing the shape of live CHO cells after 0 minutes of incubation with Compound 1 (500 nM).

FIG. 23B depicts a time-lapse phase-contrast image showing the shape of live CHO cells after 35 minutes of incubation with Compound 1 (500 nM).

FIG. 23C depicts a time-lapse phase-contrast image showing the shape of live CHO cells after 70 minutes of incubation with Compound 1 (500 nM).

FIG. 23D depicts a time-lapse phase-contrast image showing the shape of live CHO cells after 105 minutes of incubation with Compound 1 (500 nM).

FIG. 23E depicts a time-lapse phase-contrast image showing the shape of live CHO cells after 140 minutes of incubation with Compound 1 (500 nM).

FIG. 23F depicts a time-lapse phase-contrast image showing the shape of live CHO cells after 175 minutes of incubation with Compound 1 (500 nM).

FIG. 23G depicts a time-lapse phase-contrast image showing the shape of live CHO cells after 210 minutes of incubation with Compound 1 (500 nM).

FIG. 23H depicts a time-lapse phase-contrast image showing the shape of live CHO cells after 0 minutes of incubation with Velcade (100 nM).

FIG. 23I depicts a time-lapse phase-contrast image showing the shape of live CHO cells after 35 minutes of incubation with Velcade (100 nM).

FIG. 23J depicts a time-lapse phase-contrast image showing the shape of live CHO cells after 70 minutes of incubation with Velcade (100 nM).

FIG. 23K depicts a time-lapse phase-contrast image showing the shape of live CHO cells after 105 minutes of incubation with Velcade (100 nM).

FIG. 23L depicts a time-lapse phase-contrast image showing the shape of live CHO cells after 140 minutes of incubation with Velcade (100 nM).

FIG. 23M depicts a time-lapse phase-contrast image showing the shape of live CHO cells after 175 minutes of incubation with Velcade (100 nM).

FIG. 23N depicts a time-lapse phase-contrast image showing the shape of live CHO cells after 210 minutes of incubation with Velcade (100 nM).

FIG. 23O depicts a time-lapse phase-contrast image showing the shape of live CHO cells after 0 minutes of incubation with Compound 1 (500 nM) plus Taxol (10 nM).

FIG. 23P depicts a time-lapse phase-contrast image showing the shape of live CHO cells after 35 minutes of incubation with Compound 1 (500 nM) plus Taxol (10 nM).

FIG. 23Q depicts a time-lapse phase-contrast image showing the shape of live CHO cells after 70 minutes of incubation with Compound 1 (500 nM) plus Taxol (10 nM).

FIG. 23R depicts a time-lapse phase-contrast image showing the shape of live CHO cells after 105 minutes of incubation with Compound 1 (500 nM) plus Taxol (10 nM).

FIG. 23S depicts a time-lapse phase-contrast image showing the shape of live CHO cells after 140 minutes of incubation with Compound 1 (500 nM) plus Taxol (10 nM).

FIG. 23T depicts a time-lapse phase-contrast image showing the shape of live CHO cells after 175 minutes of incubation with Compound 1 (500 nM) plus Taxol (10 nM).

FIG. 23U depicts a time-lapse phase-contrast image showing the shape of live CHO cells after 210 minutes of incubation with Compound 1 (500 nM) plus Taxol (10 nM).

FIG. 23V depicts a time-lapse phase-contrast image showing the shape of live CHO cells after 0 minutes of incubation with Taxol (10 nM).

FIG. 23W depicts a time-lapse phase-contrast image showing the shape of live CHO cells after 35 minutes of incubation with Taxol (10 nM).

FIG. 23X depicts a time-lapse phase-contrast image showing the shape of live CHO cells after 70 minutes of incubation with Taxol (10 nM).

FIG. 23Y depicts a time-lapse phase-contrast image showing the shape of live CHO cells after 105 minutes of incubation with Taxol (10 nM).

FIG. 23Z depicts a time-lapse phase-contrast image showing the shape of live CHO cells after 140 minutes of incubation with Taxol (10 nM).

FIG. 23A′ depicts a time-lapse phase-contrast image showing the shape of live CHO cells after 175 minutes of incubation with Taxol (10 nM).

FIG. 23B′ depicts a time-lapse phase-contrast image showing the shape of live CHO cells after 210 minutes of incubation with Taxol (10 nM).

FIG. 23C′ depicts a time-lapse phase-contrast image showing the shape of live CHO cells after 480 minutes of incubation with Taxol (10 nM).

FIG. 24A depicts a fluorescent (α-tubulin-YFP) image of CHO cells after treatment with 10 μM Lactacystin. The arrows indicate sites of accumulation of α-tubulin-YFP in the nucleus.

FIG. 24B depicts a corresponding phase-contrast image of the CHO cells shown in FIG. 24A after treatment with 10 μM Lactacystin. The arrows indicate sites of accumulation of α-tubulin-YFP in the nucleus.

FIG. 24C depicts a fluorescent (α-tubulin-YFP) image of CHO cells after treatment with 10 μM MG132. The arrows indicate sites of accumulation of α-tubulin-YFP in the nucleus.

FIG. 24D depicts a corresponding phase-contrast image of the CHO cells shown in FIG. 24C after treatment with 10 μM 10 μM MG132. The arrows indicate sites of accumulation of α-tubulin-YFP in the nucleus.

FIG. 24E depicts a fluorescent (α-tubulin-YFP) image of CHO cells after treatment with 10 μM clasto-Lactacystin β-Lactone (cLβL). The arrows indicate sites of accumulation of α-tubulin-YFP in the nucleus.

FIG. 24F depicts a corresponding phase-contrast image of the CHO cells shown in FIG. 24E after treatment with 10 μM clasto-Lactacystin β-Lactone (cLβL). The arrows indicate sites of accumulation of α-tubulin-YFP in the nucleus.

FIG. 24G depicts a fluorescent (α-tubulin-YFP) image of CHO cells after treatment with 10 μM Epoxomicin. The arrows indicate sites of accumulation of α-tubulin-YFP in the nucleus.

FIG. 24H depicts a corresponding phase-contrast image of the CHO cells shown in FIG. 24G after treatment with 10 μM Epoxomicin. The arrows indicate sites of accumulation of α-tubulin-YFP in the nucleus.

FIG. 24I depicts a fluorescent (α-tubulin-YFP) image of CHO cells after treatment with 10 μM MG-115. The arrows indicate sites of accumulation of α-tubulin-YFP in the nucleus.

FIG. 24J depicts a corresponding phase-contrast image of the CHO cells shown in FIG. 24I after treatment with 10 μM MG-115. The arrows indicate sites of accumulation of α-tubulin-YFP in the nucleus.

FIG. 24K depicts a fluorescent (α-tubulin-YFP) image of CHO cells after treatment with 500 nM Compound 1. The arrows indicate sites of accumulation of α-tubulin-YFP in the nucleus.

FIG. 24L depicts a corresponding phase-contrast image of the CHO cells shown in FIG. 24K after treatment with 500 nM Compound 1. The arrows indicate sites of accumulation of α-tubulin-YFP in the nucleus.

FIG. 25A depicts a fluorescent image showing α-tubulin-YFP localization in CHO cells that have been treated for 8 hours with Compound 1 (50 nM). The red arrows indicate accumulation of α-tubulin-YFP at centrosomes.

FIG. 25B depicts a fluorescent image showing α-tubulin-YFP localization in CHO cells that have been treated for 8 hours with Drug-V (Velcade; 50 nM).

FIG. 25C depicts a fluorescent image showing α-tubulin-YFP localization in CHO cells that have been treated for 8 hours with Drug-V (Velcade; 100 nM).

FIG. 25D depicts a fluorescent image showing α-tubulin-YFP localization in CHO cells that have been treated for 8 hours with Drug-V (Velcade; 500 nM). The red arrows show accumulation of α-tubulin-YFP at centrosomes and the aqua arrows show accumulation α-tubulin-YFP in the nucleus.

FIG. 26A depicts a fluorescent image of α-tubulin-YFP localization in CHO cells that have been treated for 20 hours with Compound 1 (50 nM). The red arrows show accumulation of α-tubulin-YFP at centrosomes.

FIG. 26B depicts a fluorescent image of α-tubulin-YFP localization in CHO cells that have been treated for 20 hours with Compound 1 (500 nM). The red arrows show accumulation of α-tubulin-YFP at centrosomes.

FIG. 26C depicts a fluorescent image of α-tubulin-YFP localization in CHO cells that have been treated for 20 hours with Drug-V (Velcade; 10 nM).

FIG. 26D depicts a fluorescent image of α-tubulin-YFP localization in CHO cells that have been treated for 20 hours with Drug-V (Velcade; 50 nM). The red arrows show accumulation of α-tubulin-YFP at centrosomes and the aqua arrows show accumulation of α-tubulin-YFP in the nucleus.

FIG. 26E depicts a fluorescent image of α-tubulin-YFP localization in CHO cells that have been treated for 20 hours with Drug-V (Velcade; 500 nM). The red arrows show accumulation of α-tubulin-YFP at centrosomes and the aqua arrows show accumulation of α-tubulin-YFP in the nucleus.

FIG. 26F depicts a fluorescent image of α-tubulin-YFP localization in CHO cells that have been treated for 20 hours with Compound 3 (5 μM).

FIG. 27 is a Western blot of cell lysates from MDA-435 breast cancer cells following treatment with various compounds for either 6 or 24 hours. Each compound (Compound 1, Compound 1+Taxol (labeled “Comb”), MG132, ALLN and Lactacystin (labeled “LACT”)) was tested individually at a concentration of 0.5 μM. DMSO and Taxol treatments served as controls. Induction of Hsp70 and GAPDH proteins was monitored using Hsp70 and GAPDH antibodies, respectively.

FIG. 28 is a Western blot that was probed with an antibody that specifically recognizes multi-ubiquitin chains. Cell lysates were prepared from MDA-435 cells treated for either 6 or 24 hours with Taxol, Compound 1, Taxol+Compound 1 (labeled “Combo”), MG-132, ALLN or Lactacystin (labeled “LACT”) (0.5 μM each). DMSO-treated samples were included as controls. Dark smears between approximately 80 and 220 kDa are indicative of the accumulation of multi-ubiquitinated proteins.

FIG. 29 is a Western blot that was probed with an antibody that specifically recognizes multi-ubiquitin chains. Cell lysates were prepared from MDA-435 cells treated for 6 and 24 hours with Compound 1 (0.5 μM), Taxol (0.5 μM), Taxol+Compound 1 (labeled “Combo”; 0.5 μM of each), Compound 2 (0.5 μM) or Velcade (0.5 μM and 5 nM). DMSO-treated and Mannitol-treated samples were included as controls. Dark smears between approximately 80 and 220 kDa are indicative of the accumulation of multi-ubiquitinated proteins.

FIG. 30 is a graph comparing the effects of Compound 1 to those of Compound 5 (Aurora-A kinase inhibitor VX-680; Vertex Pharmaceuticals, Inc., Cambridge, Mass.) on Aurora-A kinase activity using an in vitro ELISA assay that monitors phosphorylation of the Lats2 Aurora-A substrate. Each compound was tested at a range of concentrations between 50 nM and 50 μM.

FIG. 31 is a graph depicting the effects of Compound 1, Taxol, and a combination of the two compounds, on the kinetics of tubulin polymerization in vitro. The tested doses were: 3 μM Taxol, 0.5 μM Compound 1, 0.5 μM Compound 1+3 μM Taxol (labeled as “Taxol+Compound 1”), and 0.5 μM Compound 1+30 nM of Taxol (labeled as “low dose Taxol+Compound 1”). Sample containing no tubulin and no drug were used as controls.

FIG. 32 is a graph depicting the effects of Compound 1, Taxol, or a combination of the two compounds on polymerization of MAP-enriched tubulin in vitro. The tested doses were 0.5 μM Compound 1, 3 μM Taxol, and 0.5 μM Compound 1+3 μM Taxol. Each sample was run in duplicate.

FIG. 33A depicts a fluorescent image showing the localization of Oregon Green 488-labeled Taxol to microtubules in CHO cells. Letter designations indicate localization of Oregon Green 488-Taxol to mitotic midbodies (D) and centrosomal regions (B, C).

FIG. 33B depicts a fluorescent image showing the localization of Oregon Green 488-labeled Taxol to centrosomal regions in CHO cells.

FIG. 33C depicts a fluorescent image showing the localization of Oregon Green 488-labeled Taxol to centrosomal regions in CHO cells.

FIG. 33D depicts a fluorescent image showing the localization of Oregon Green 488-labeled Taxol to mitotic midbodies in CHO cells.

FIG. 34A depicts the localization of Oregon Green 488-labeled Taxol in HeLa cells treated with 1:1000 DMSO as a control. The arrows indicate fluorescent-Taxol at the centrosomes.

FIG. 34B depicts the localization of Oregon Green 488-labeled Taxol in HeLa cells treated with Compound 1 (0.5 μM). The arrows indicate fluorescent-Taxol at the centrosomes.

FIG. 34C depicts the localization of Oregon Green 488-labeled Taxol in CHO cells treated with DMSO as a control. The arrows indicate fluorescent-Taxol at the centrosomes.

FIG. 34D depicts the localization of Oregon Green 488-labeled Taxol in CHO cells treated with Compound 1 (0.5 μM). The arrows indicate fluorescent-Taxol at the centrosomes.

FIG. 34E depicts a fluorescent image of CHO cells treated with non-labeled Taxol (300 μM).

FIG. 34F depicts the corresponding phase-contrast image of FIG. 34E.

FIG. 35A depicts a phase contrast image of isolated centrosomes from CHO cells.

FIG. 35B depicts a fluorescent image of isolated centrosomes from CHO cells. Gamma-tubulin staining was performed to confirm the presence of centrosomes in the isolated fraction.

FIG. 35C depicts a fluorescent image of the localization of Oregon Green 488 labeled-Taxol in the centrosome of CHO cells, showing similar sizes of centrosomes to the isolated centrosomes in FIG. 35B.

FIG. 35D depicts the merged image of FIGS. 35A and B.

FIG. 36A depicts an image of a population of CRL-2261 non-Hodgkin's lymphoma cells treated with DMSO for 48 hours showing staining with calcein AM (green), a marker for live cells.

FIG. 36B depicts an image of the cells shown in FIG. 36A showing staining with ethidium homodimer (red), a marker for dead cells.

FIG. 36C depicts a merged image of the images shown in FIG. 36A and FIG. 36B.

FIG. 36D depicts an image of a population of CRL-2261 non-Hodgkin's lymphoma cells treated with 0.5 nM Compound 1 for 48 hours showing staining with calcein AM (green), a marker for live cells.

FIG. 36E depicts an image of the cells shown in FIG. 36D showing staining with ethidium homodimer (red), a marker for dead cells.

FIG. 36F depicts a merged image of the images shown in FIG. 36D and FIG. 36E.

FIG. 36G depicts an image of a population of CRL-2261 non-Hodgkin's lymphoma cells treated with 5 nM Compound 1 for 48 hours showing staining with calcein AM (green), a marker for live cells.

FIG. 36H depicts an image of the cells shown in FIG. 36G showing staining with ethidium homodimer (red), a marker for dead cells.

FIG. 36I depicts a merged image of the images shown in FIG. 36G and FIG. 36H.

FIG. 36J depicts an image of a population of CRL-2261 non-Hodgkin's lymphoma cells treated with 50 nM Compound 1 for 48 hours showing staining with calcein AM (green), a marker for live cells.

FIG. 36K depicts an image of the cells shown in FIG. 36J showing staining with ethidium homodimer (red), a marker for dead cells.

FIG. 36L depicts a merged image of the images shown in FIG. 36J and FIG. 36K.

FIG. 36M depicts an image of a population of CRL-2261 non-Hodgkin's lymphoma cells treated with 500 nM Compound 1 for 48 hours showing staining with calcein AM (green), a marker for live cells.

FIG. 36N depicts an image of the cells shown in FIG. 36M showing staining with ethidium homodimer (red), a marker for dead cells.

FIG. 36O depicts a merged image of the images shown in FIG. 36M and FIG. 36N.

FIG. 36P depicts an image of a population of CRL-2261 non-Hodgkin's lymphoma cells treated with 5000 nM Compound 1 for 48 hours showing staining with calcein AM (green), a marker for live cells.

FIG. 36Q depicts an image of the cells shown in FIG. 36P showing staining with ethidium homodimer (red), a marker for dead cells.

FIG. 36R depicts a merged image of the images shown in FIG. 36P and FIG. 36Q.

FIG. 37A depicts an image of a population of U937 histiocytic lymphoma cells treated with DMSO for 36 hours and stained with calcein AM (green), a marker for live cells.

FIG. 37B depicts an image of a population of U937 histiocytic lymphoma cells treated with DMSO for 36 hours and stained with ethidium homodimer (red), a marker for dead cells.

FIG. 37C depicts an image of a population of U937 histiocytic lymphoma cells treated with 0.5 μM Compound 1 for 36 hours and stained with calcein AM (green), a marker for live cells.

FIG. 37D depicts an image of a population of U937 histiocytic lymphoma cells treated with 0.5 μM Compound 1 for 36 hours and stained with ethidium homodimer (red), a marker for dead cells.

FIG. 37E depicts an image of a population of U937 histiocytic lymphoma cells treated with 5 μM Compound 1 for 36 hours and stained with calcein AM (green), a marker for live cells.

FIG. 37F depicts an image of a population of U937 histiocytic lymphoma cells treated with 5 μM Compound 1 for 36 hours and stained with ethidium homodimer (red), a marker for dead cells.

FIG. 38 is a graph depicting the effect of the compounds ALLN, MG132, Lactacystin and Compound 1 on proteasome activity using an in vitro assay. Activity was monitored at various time points for 108 minutes after treatment. Concentrations of 5 nM for all drugs (Compound 1, ALLN, MG132, and Lactacystin) were tested. Controls include DMSO (1:1, labeled as “Control”) as well as no enzyme control (labeled as “No Enzyme”).

### DETAILED DESCRIPTION OF THE INVENTION

In one embodiment, the invention is a compound that exhibits one or more of a subset of properties. The compounds are able to: i) disrupt organization of an actin cytoskeleton of a cell; ii) disrupt organization of a microtubule network of a cell; iii) induce accumulation of tubulin at centrosomes but not induce accumulation of tubulin in a nucleus of a cell; iv) induce accumulation of tubulin at centrosomes at a concentration of 500 nM or less within four hours; v) induce accumulation of Hsp70 but only possess weak-to-moderate proteasome inhibitory activity; and/or vi) not possess proteasome inhibitory activity when assayed on purified proteasomes. In this embodiment, the compound is not a bis(thio-hydrazide amide) represented by Structural Formula (I):

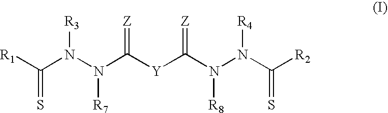

wherein Y is a covalent bond or a substituted or unsubstituted straight chained hydrocarbyl group, or, Y, taken together with both >C═Z groups to which it is bonded, is a substituted or unsubstituted aromatic group;

R₁-R₄ are independently —H, an aliphatic group, a substituted aliphatic group, an aryl group or a substituted aryl group, or R₁ and R₃ taken together with the carbon and nitrogen atoms to which they are bonded, and/or R₂ and R₄ taken together with the carbon and nitrogen atoms to which they are bonded, form a non-aromatic heterocyclic ring optionally fused to an aromatic ring;

R₅ and R₆ are each independently —H, an aliphatic or substituted aliphatic group, or R₅ is —H and R₆ is a substituted or unsubstituted aryl group, or, R₅ and R₆, taken together, are a C2-C6 substituted or unsubstituted alkylene group;

R₇-R₈ are independently —H, an aliphatic group, a substituted aliphatic group, an aryl group or a substituted aryl group; and

Z is ═O or ═S.

As used herein, the bis(thio-hydrazide amides) that are employed in particular embodiments, or excluded from other embodiments, are represented by Structural Formula (I). 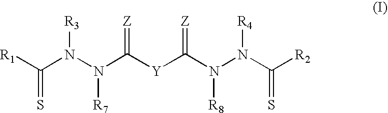
wherein Y is a covalent bond or a substituted or unsubstituted straight chained hydrocarbyl group, or, Y, taken together with both >C═Z groups to which it is bonded, is a substituted or unsubstituted aromatic group. Preferably, Y is a covalent bond or —C(R₅R₆)—.

R₁-R₄ are independently —H, an aliphatic group, a substituted aliphatic group, an aryl group or a substituted aryl group, or R₁ and R₃ taken together with the carbon and nitrogen atoms to which they are bonded, and/or R₂ and R₄ taken together with the carbon and nitrogen atoms to which they are bonded, form a non-aromatic heterocyclic ring optionally fused to an aromatic ring. Preferably R₁ and R₂ are the same and R₃ and R₄ are the same.

R₅ and R₆ are each independently —H, an aliphatic or substituted aliphatic group, or R₅ is —H and R₆ is a substituted or unsubstituted aryl group, or, R₅ and R₆, taken together, are a C2-C6 substituted or unsubstituted alkylene group.

R₇-R₈ are independently —H, an aliphatic group, a substituted aliphatic group, an aryl group or a substituted aryl group. Preferably, R₇ and R₈ are the same.

Z is ═O or ═S.

A “straight chained hydrocarbyl group” is an alkylene group, i.e., —(CH₂)_{y}—, with one, or more (preferably one) internal methylene groups optionally replaced with a linkage group. y is a positive integer (e.g., between 1 and 10), preferably between 1 and 6 and more preferably 1 or 2. A “linkage group” refers to a functional group which replaces a methylene in a straight chained hydrocarbyl. Examples of suitable linkage groups include a ketone (—C(O)—), alkene, alkyne, phenylene, ether (—O—), thioether (—S—), or amine (—N(R^{a})—), wherein R^{a} is defined below. A preferred linkage group is —C(R₅R₆)—, wherein R₅ and R₆ are defined above. Suitable substitutents for an alkylene group and a hydrocarbyl group are those which do not substantially interfere with the activities described herein (e.g., proteasome inhibiting activity) of the disclosed compounds. R₅ and R₆ are preferred substituents for an alkylene or hydrocarbyl group represented by Y.

An aliphatic group is a straight chained, branched or cyclic non-aromatic hydrocarbon which is completely saturated or which contains one or more units of unsaturation. Typically, a straight chained or branched aliphatic group has from 1 to about 20 carbon atoms, preferably from 1 to about 10, and a cyclic aliphatic group has from 3 to about 10 carbon atoms, preferably from 3 to about 8. An aliphatic group is preferably a straight chained or branched alkyl group, e.g., methyl, ethyl, n-propyl, iso-propyl, n-butyl, sec-butyl, tert-butyl, pentyl, hexyl, pentyl or octyl, or a cycloalkyl group with 3 to about 8 carbon atoms. A C1-C20 straight chained or branched alkyl group or a C3-C8 cyclic alkyl group is also referred to as a “lower alkyl” group.

The term “aromatic group” may be used interchangeably with “aryl,” “aryl ring,” “aromatic ring,” “aryl group” and “aromatic group.” Aromatic groups include carbocyclic aromatic groups, such as phenyl, naphthyl, and anthracyl, and heteroaryl groups, such as imidazolyl, thienyl, furanyl, pyridyl, pyrimidy, pyranyl, pyrazolyl, pyrroyl, pyrazinyl, thiazole, oxazolyl, and tetrazole. The term “heteroaryl group” may be used interchangeably with “heteroaryl,” “heteroaryl ring,” “heteroaromatic ring” and “heteroaromatic group.” The term “heteroaryl,” as used herein, means a mono-or multi-cyclic aromatic heterocycle which comprises at least one heteroatom, such as nitrogen, sulfur and oxygen, but may include 1, 2, 3 or 4 heteroatoms per ring. Aromatic groups also include fused polycyclic aromatic ring systems in which a carbocyclic aromatic ring or heteroaryl ring is fused to one or more other heteroaryl rings. Examples include benzothienyl, benzofuranyl, indolyl, quinolinyl, benzothiazole, benzooxazole, benzimidazole, quinolinyl, isoquinolinyl and isoindolyl.

The term “arylene” refers to an aryl group which is connected to the remainder of the molecule by two other bonds. By way of example, the structure of a 1,4-phenylene group is shown below:

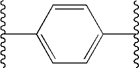

Substituents for an arylene group are as described below for an aryl group.

Non-aromatic heterocyclic rings are non-aromatic rings which include one or more heteroatoms, such as nitrogen, oxygen or sulfur, in the ring. The ring can be five, six, seven or eight-membered. Examples include tetrahydrofuranyl, tetrahyrothiophenyl, morpholino, thiomorpholino, pyrrolidinyl, piperazinyl, piperidinyl, and thiazolidinyl.

Suitable substituents on an aliphatic group (including an alkylene group), non-aromatic heterocyclic group, benzylic or aryl group (carbocyclic and heteroaryl) are those which do not substantially interfere with one or more of the activities (e.g., proteasome inhibiting activity) of the disclosed compounds as described herein. A substituent substantially interferes with one or more of the activities when the activity (e.g., proteasome inhibiting activity) is reduced by more than about 50% in a compound with the substituent as compared to a compound without the substituent. Examples of suitable substituents include —R^{a}, —OH, —Br, —Cl, —I, —F, —OR^{a}, —O—COR^{a}, —COR^{a}, —CN, —NO₂, —COOH, —SO₃H, —NH₂, —NHR^{a}, —N(R^{a}R^{b}), —COOR^{a}, —CHO, —CONH₂, —CONHR^{a}, —CON(R^{a}R^{b}), —NHCOR^{a}, —NR^{c}COR^{a}, —NHCONH₂, —NHCONR^{a}H, —NHCON(R^{a}R^{b}), —NR^{c}CONH₂, —NR^{c}CONR^{a}H, —NR^{c}CON(R^{a}R^{b}), —C(═NH)—NH₂, —C(═NH)—NHR^{a}, —C(═NH)—N(R^{a}R^{b}), —C(═NR^{c})—NH₂, —C(═NR^{c})—NHR^{a}, —C(═NR^{c})—N(R^{a}R^{b}), —NH—C(═NH)—NH₂, —NH—C(═NH)—NHR^{a}, —NH—C(═NH)—N(R^{a}R^{b}), —NH—C(═NR^{c})—NH₂, —NH—C(═NR^{c})—NHR^{a}, —NH—C(═NR^{c})—N(R^{a}R^{b}), —NR^{d}H—C(═NH)—NH₂, —NR^{d}—C(═NH)—NHR^{a}, —NR^{d}—C(═NH)—N(R^{a}R^{b}), —NR^{d}—C(═NR^{c})NH₂, —NR^{d}—C(═NR^{c})—NHR^{a}, —NR^{d}—C(═NR^{c})—N(R^{a}R^{b}), —NHNH₂—NHNHR^{a}, —NHR^{a}R^{b}, —SO₂NH₂, —SO₂NHR^{a}, —SO₂NR^{a}R^{b}, —CH═CHR^{a}, —CH═CR^{a}R^{b}, —CR^{c}═CR^{a}R^{b}, —CR^{c}═CHR^{a}, —CR^{c}═CR^{a}R^{b}, —CCR^{a}, —SH, —SR^{a}, —S(O)R^{a}, —S(O)₂R^{a}. R^{a}-R^{d} are each independently an alkyl group, aromatic group, non-aromatic heterocyclic group or —N(R^{a}R^{b}), taken together, form an optionally substituted non-aromatic heterocyclic group. The alkyl, aromatic and non-aromatic heterocyclic group represented by R^{a}-R^{d} and the non-aromatic heterocyclic group represented by —N(R^{a}R^{b}) are each optionally and independently substituted with one or more groups represented by R^{#}.

R^{#} is R⁺, —OR⁺, —O(haloalkyl), —SR⁺, —NO₂, —CN, —NCS, —N(R⁺)₂, —NHCO₂R⁺, —NHC(O)R⁺, —NHNHC(O)R⁺, —NHC(O)N(R⁺)₂, —NHNHC(O)N(R⁺)₂, —NHNHCO₂R⁺, —C(O)C(O)R⁺, —C(O)CH₂C(O)R⁺, —CO₂R⁺, —C(O)R⁺, —C(O)N(R⁺)₂, —OC(O)R⁺, —OC(O)N(R+)₂, —S(O)₂R⁺, —SO₂N(R⁺)₂, —S(O)R⁺, —NHSO₂N(R⁺)₂, —NHSO₂R⁺, —C(═S)N(R⁺)₂, or —C(═NH)—N(R⁺)₂.

R⁺ is —H, a C1-C4 alkyl group, a monocyclic heteroaryl group, a non-aromatic heterocyclic group or a phenyl group optionally substituted with alkyl, haloalkyl, alkoxy, haloalkoxy, halo, —CN, —NO₂, amine, alkylamine or dialkylamine. Optionally, the group —N(R⁺)₂ is a non-aromatic heterocyclic group, provided that non-aromatic heterocyclic groups represented by R⁺ and —N(R⁺)₂ that comprise a secondary ring amine are optionally acylated or alkylated.

Preferred substituents for a phenyl group, including phenyl groups represented by R₁-R₄, include C1-C4 alkyl, C1-C4 alkoxy, C1-C4 haloalkyl, C1-C4 haloalkoxy, phenyl, benzyl, pyridyl, —OH, —NH₂, —F, —Cl, —Br, —I, —NO₂ or —CN.

Preferred substituents for a cycloalkyl group, including cycloalkyl groups represented by R₁ and R₂, are alkyl groups, such as a methyl or ethyl group.

In one embodiment, Y in Structural Formula I is a covalent bond, —C(R₅R₆)—, —(CH₂CH₂)—, trans-(CH═CH)—, cis-(CH═CH)— or —(C≡C)— group, preferably —C(R₅R₆)—. R₁-R₄ and R₇-R₈ are as described above for Structural Formula I. R₅ and R₆ are each independently —H, an aliphatic or substituted aliphatic group, or R₅ is —H and R₆ is an optionally substituted aryl group, or, R₅ and R₆, taken together, are an optionally substituted C2-C6 alkylene group. The pharmaceutically-acceptable cation is as described in detail below.

In specific embodiments, Y taken together with both >C═Z groups to which it is bonded, is an optionally substituted aromatic group. In this instance, certain bis(thio-hydrazide amides) are represented by Structural Formula II: 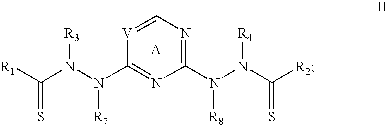
wherein Ring A is substituted or unsubstituted and V is —CH— or —N—. The other variables in Structural Formula II are as described herein for Structural Formula I or III.

In particular embodiments, the bis(thio-hydrazide amides) are represented by Structural Formula III or IV: 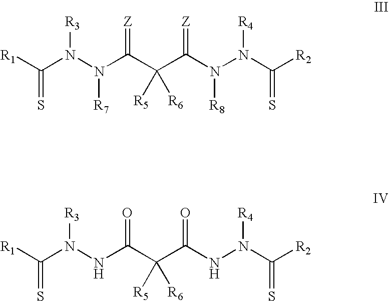
R₁-R₈ in Structural Formulas III and IV are as described above for Structural Formula I.

In Structural Formulas I-IV, R₁ and R₂ are the same or different and/or R₃ and R₄ are the same or different; preferably, R₁ and R₂ are the same and R₃ and R₄ are the same. In Structural Formulas I, III and IV, Z is preferably O. Typically in Structural Formulas I, III and IV, Z is O; R₁ and R₂ are the same; and R₃ and R₄ are the same. More preferably, Z is O; R₁ and R₂ are the same; R₃ and R₄ are the same, and R₇ and R₈ are the same.

In other embodiments, the bis(thio-hydrazide amides) are represented by Structural Formulas III or IV: R₁ and R₂ are each an optionally substituted aryl group, preferably an optionally substituted phenyl group; R₃ and R₄ are each an optionally substituted aliphatic group, preferably an alkyl group, more preferably, methyl or ethyl; and R₅ and R₆ are as described above, but R₅ is preferably —H and R₆ is preferably —H, an aliphatic or substituted aliphatic group.

Alternatively, R₁ and R₂ are each an optionally substituted aryl group; R₃ and R₄ are each an optionally substituted aliphatic group; R₅ is —H; and R₆ is —H, an aliphatic or substituted aliphatic group. Preferably, R₁ and R₂ are each an optionally substituted aryl group; R₃ and R₄ are each an alkyl group; and R₅ is —H and R₆ is —H or methyl. Even more preferably, R₁ and R₂ are each an optionally substituted phenyl group; R₃ and R₄ are each methyl or ethyl; and R₅ is —H and R₆ is —H or methyl. Suitable substituents for an aryl group represented by R₁ and R₂ and an aliphatic group represented by R₃, R₄ and R₆ are as described below for aryl and aliphatic groups.

In another embodiment, the bis(thio-hydrazide amides) are represented by Structural Formulas III and IV: R₁ and R₂ are each an optionally substituted aliphatic group, preferably a C3-C8 cycloalkyl group optionally substituted with at least one alkyl group, more preferably cyclopropyl or 1-methylcyclopropyl; R₃ and R₄ are as described above for Structural Formula I, preferably both an optionally substituted alkyl group; and R₅ and R₆ are as described above, but R₅ is preferably —H and R₆ is preferably —H, an aliphatic or substituted aliphatic group, more preferably —H or methyl.

Alternatively, the bis(thio-hydrazide amides) are represented by Structural Formulas III or IV: R₁ and R₂ are each an optionally substituted aliphatic group; R₃ and R₄ are as described above for Structural Formula I, preferably both an optionally substituted alkyl group; and R₅ is —H and R₆ is —H or an optionally substituted aliphatic group. Preferably, R₁ and R₂ are both a C3-C8 cycloalkyl group optionally substituted with at least one alkyl group; R₃ and R₄ are both as described above for Structural Formula I, preferably an alkyl group; and R₅ is —H and R₆ is —H or an aliphatic or substituted aliphatic group. More preferably, R₁ and R₂ are both a C3-C8 cycloalkyl group optionally substituted with at least one alkyl group; R₃ and R₄ are both an alkyl group; and R₅ is —H and R₆ is —H or methyl. Even more preferably, R₁ and R₂ are both cyclopropyl or 1-methylcyclopropyl; R₃ and R₄ are both an alkyl group, preferably methyl or ethyl; and R₅ is —H and R₆ is —H or methyl.

In specific embodiments, the bis(thio-hydrazide amides) are represented by Structural Formula IV:

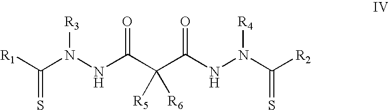

wherein: R₁ and R₂ are both phenyl, R₃ and R₄ are both methyl, and R₅ and R₆ are both —H; R₁ and R₂ are both phenyl, R₃ and R₄ are both ethyl, and R₅ and R₆ are both —H; R₁ and R₂ are both 4-cyanophenyl, R₃ and R₄ are both methyl, R₅ is methyl, and R₆ is —H; R₁ and R₂ are both 4-methoxyphenyl, R₃ and R₄ are both methyl, and R₅ and R₆ are both —H; R₁ and R₂ are both phenyl, R₃ and R₄ are both methyl, R₅ is methyl, and R₆ is —H; R₁ and R₂ are both phenyl, R₃ and R₄ are both ethyl, R₅ is methyl, and R₆ is —H; R₁ and R₂ are both 4-cyanophenyl, R₃ and R₄ are both methyl, and R₅ and R₆ are both —H; R₁ and R₂ are both 2,5-dimethoxyphenyl, R₃ and R₄ are both methyl, and R₅ and R₆ are both —H; R₁ and R₂ are both 2,5-dimethoxyphenyl, R₃ and R₄ are both methyl, R₅ is methyl, and R₆ is —H; R₁ and R₂ are both 3-cyanophenyl, R₃ and R₄ are both methyl, and R₅ and R₆ are both —H; R₁ and R₂ are both 3-fluorophenyl, R₃ and R₄ are both methyl, and R₅ and R₆ are both —H; R₁ and R₂ are both 4-chlorophenyl, R₃ and R₄ are both methyl, R₅ is methyl, and R₆ is —H; R₁ and R₂ are both 2-dimethoxyphenyl, R₃ and R₄ are both methyl, and R₅ and R₆ are both —H; R₁ and R₂ are both 3-methoxyphenyl, R₃ and R₄ are both methyl, and R₅ and R₆ are both —H; R₁ and R₂ are both 2,3-dimethoxyphenyl, R₃ and R₄ are both methyl, and R₅ and R₆ are both —H; R₁ and R₂ are both 2,3-dimethoxyphenyl, R₃ and R₄ are both methyl, R₅ is methyl, and R₆ is —H; R₁ and R₂ are both 2,5-difluorophenyl, R₃ and R₄ are both methyl, and R₅ and R₆ are both —H; R₁ and R₂ are both 2,5-difluorophenyl, R₃ and R₄ are both methyl, R₅ is methyl, and R₆ is —H; R₁ and R₂ are both 2,5-dichlorophenyl, R₃ and R₄ are both methyl, and R₅ and R₆ are both —H; R₁ and R₂ are both 2,5-dimethylphenyl, R₃ and R₄ are both methyl, and R₅ and R₆ are both —H; R₁ and R₂ are both 2,5-dimethoxyphenyl, R₃ and R₄ are both methyl, and R₅ and R₆ are both —H; R₁ and R₂ are both phenyl, R₃ and R₄ are both methyl, and R₅ and R₆ are both —H; R₁ and R₂ are both 2,5-dimethoxyphenyl, R₃ and R₄ are both methyl, R₅ is methyl, and R₆ is —H; R₁ and R₂ are both cyclopropyl, R₃ and R₄ are both methyl, and R₅ and R₆ are both —H; R₁ and R₂ are both cyclopropyl, R₃ and R₄ are both ethyl, and R₅ and R₆ are both —H; R₁ and R₂ are both cyclopropyl, R₃ and R₄ are both methyl, R₅ is methyl, and R₆ is —H; R₁ and R₂ are both 1-methylcyclopropyl, R₃ and R₄ are both methyl, and R₅ and R₆ are both —H; R₁ and R₂ are both 1-methylcyclopropyl, R₃ and R₄ are both methyl, R₅ is methyl and R₆ is —H; R₁ and R₂ are both 1-methylcyclopropyl, R₃ and R₄ are both methyl, R₅ is ethyl, and R₆ is —H; R₁ and R₂ are both 1-methylcyclopropyl, R₃ and R₄ are both methyl, R₅ is n-propyl, and R₆ is —H; R₁ and R₂ are both 1-methylcyclopropyl, R₃ and R₄ are both methyl, and R₅ and R₆ are both methyl; R₁ and R₂ are both 1-methylcyclopropyl, R₃ and R₄ are both ethyl, and R₅ and R₆ are both —H; R₁ and R₂ are both 1-methylcyclopropyl, R₃ is methyl, R₄ is ethyl, and R₅ and R₆ are both —H; R₁ and R₂ are both 2-methylcyclopropyl, R₃ and R₄ are both methyl, and R₅ and R₆ are both —H; R₁ and R₂ are both 2-phenylcyclopropyl, R₃ and R₄ are both methyl, and R₅ and R₆ are both —H; R₁ and R₂ are both 1-phenylcyclopropyl, R₃ and R₄ are both methyl, and R₅ and R₆ are both —H; R₁ and R₂ are both cyclobutyl, R₃ and R₄ are both methyl, and R₅ and R₆ are both —H; R₁ and R₂ are both cyclopentyl, R₃ and R₄ are both methyl, and R₅ and R₆ are both —H; R₁ and R₂ are both cyclohexyl, R₃ and R₄ are both methyl, and R₅ and R₆ are both —H; R₁ and R₂ are both cyclohexyl, R₃ and R₄ are both phenyl, and R₅ and R₆ are both —H; R₁ and R₂ are both methyl, R₃ and R₄ are both methyl, and R₅ and R₆ are both —H; R₁ and R₂ are both methyl, R₃ and R₄ are both t-butyl, and R₅ and R₆ are both —H; R₁ and R₂ are both methyl, R₃ and R₄ are both phenyl, and R₅ and R₆ are both —H; R₁ and R₂ are both t-butyl, R₃ and R₄ are both methyl, and R₅ and R₆ are both —H; R₁ and R₂ are ethyl, R₃ and R₄ are both methyl, and R₅ and R₆ are both —H; or R₁ and R₂ are both n-propyl, R₃ and R₄ are both methyl, and R₅ and R₆ are both —H.

In specific embodiments, the bis(thio-hydrazide amides) are represented by Structural Formula V: 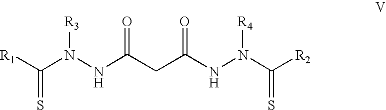
wherein: R₁ and R₂ are both phenyl, and R₃ and R₄ are both o-CH₃-phenyl; R₁ and R₂ are both o-CH₃C(O)O-phenyl, and R₃ and R₄ are phenyl; R₁ and R₂ are both phenyl, and R₃ and R₄ are both methyl; R₁ and R₂ are both phenyl, and R₃ and R₄ are both ethyl; R₁ and R₂ are both phenyl, and R₃ and R₄ are both n-propyl; R₁ and R₂ are both p-cyanophenyl, and R₃ and R₄ are both methyl; R₁ and R₂ are both p-nitro phenyl, and R₃ and R₄ are both methyl; R₁ and R₂ are both 2,5-dimethoxyphenyl, and R₃ and R₄ are both methyl; R₁ and R₂ are both phenyl, and R₃ and R₄ are both n-butyl; R₁ and R₂ are both p-chlorophenyl, and R₃ and R₄ are both methyl; R₁ and R₂ are both 3-nitrophenyl, and R₃ and R₄ are both methyl; R₁ and R₂ are both 3-cyanophenyl, and R₃ and R₄ are both methyl; R₁ and R₂ are both 3-fluorophenyl, and R₃ and R₄ are both methyl; R₁ and R₂ are both 2-furanyl, and R₃ and R₄ are both phenyl; R₁ and R₂ are both 2-methoxyphenyl, and R₃ and R₄ are both methyl; R₁ and R₂ are both 3-methoxyphenyl, and R₃ and R₄ are both methyl; R₁ and R₂ are both 2,3-dimethoxyphenyl, and R₃ and R₄ are both methyl; R₁ and R₂ are both 2-methoxy-5-chlorophenyl, and R₃ and R₄ are both ethyl; R₁ and R₂ are both 2,5-difluorophenyl, and R₃ and R₄ are both methyl; R₁ and R₂ are both 2,5-dichlorophenyl, and R₃ and R₄ are both methyl; R₁ and R₂ are both 2,5-dimethylphenyl, and R₃ and R₄ are both methyl; R₁ and R₂ are both 2-methoxy-5-chlorophenyl, and R₃ and R₄ are both methyl; R₁ and R₂ are both 3,6-dimethoxyphenyl, and R₃ and R₄ are both methyl; R₁ and R₂ are both phenyl, and R₃ and R₄ are both 2-ethylphenyl; R₁ and R₂ are both 2-methyl-5-pyridyl, and R₃ and R₄ are both methyl; or R₁ is phenyl; R₂ is 2,5-dimethoxyphenyl, and R₃ and R₄ are both methyl; R₁ and R₂ are both methyl, and R₃ and R₄ are both p-CF₃-phenyl; R₁ and R₂ are both methyl, and R₃ and R₄ are both o-CH₃-phenyl; R₁ and R₂ are both —(CH₂)₃COOH; and R₃ and R₄ are both phenyl; R₁ and R₂ are both represented by the following structural formula: 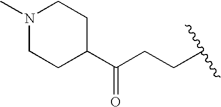
, and R₃ and R₄ are both phenyl; R₁ and R₂ are both n-butyl, and R₃ and R₄ are both phenyl; R₁ and R₂ are both n-pentyl, R₃ and R₄ are both phenyl; R₁ and R₂ are both methyl, and R₃ and R₄ are both 2-pyridyl; R₁ and R₂ are both cyclohexyl, and R₃ and R₄ are both phenyl; R₁ and R₂ are both methyl, and R₃ and R₄ are both 2-ethylphenyl; R₁ and R₂ are both methyl, and R₃ and R₄ are both 2,6-dichlorophenyl; R₁-R₄ are all methyl; R₁ and R₂ are both methyl, and R₃ and R₄ are both t-butyl; R₁ and R₂ are both ethyl, and R₃ and R₄ are both methyl; R₁ and R₂ are both t-butyl, and R₃ and R₄ are both methyl; R₁ and R₂ are both cyclopropyl, and R₃ and R₄ are both methyl; R₁ and R₂ are both cyclopropyl, and R₃ and R₄ are both ethyl; R₁ and R₂ are both 1-methylcyclopropyl, and R₃ and R₄ are both methyl; R₁ and R₂ are both 2-methylcyclopropyl, and R₃ and R₄ are both methyl; R₁ and R₂ are both 1-phenylcyclopropyl, and R₃ and R₄ are both methyl; R₁ and R₂ are both 2-phenylcyclopropyl, and R₃ and R₄ are both methyl; R₁ and R₂ are both cyclobutyl, and R₃ and R₄ are both methyl; R₁ and R₂ are both cyclopentyl, and R₃ and R₄ are both methyl; R₁ is cyclopropyl, R₂ is phenyl, and R₃ and R₄ are both methyl.

Preferred examples of bis(thio-hydrazide amides) include Compounds (1′)-(18′) and pharmaceutically-acceptable salts and solvates thereof:

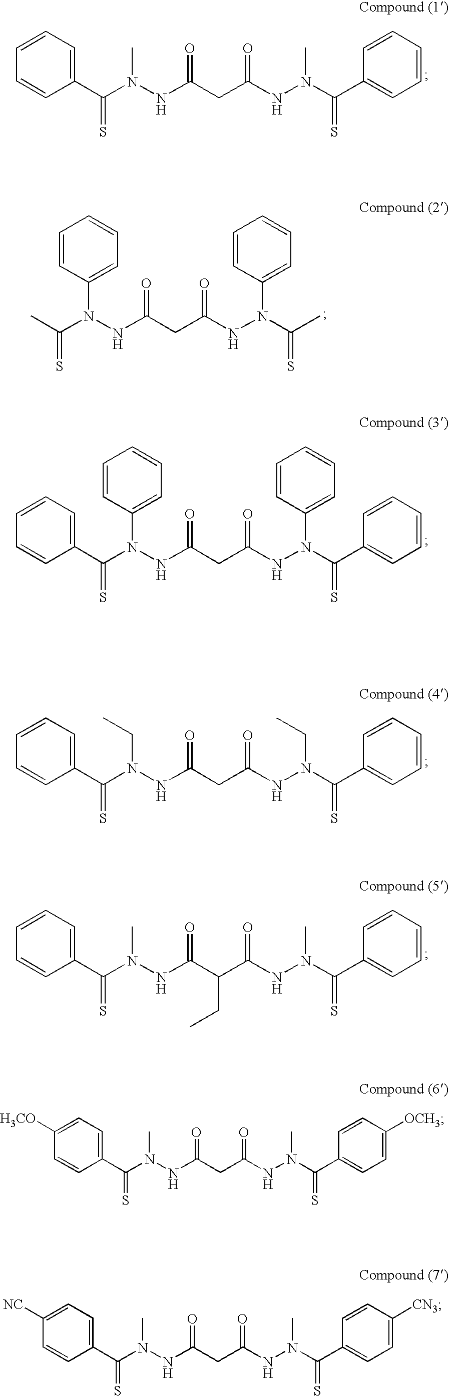

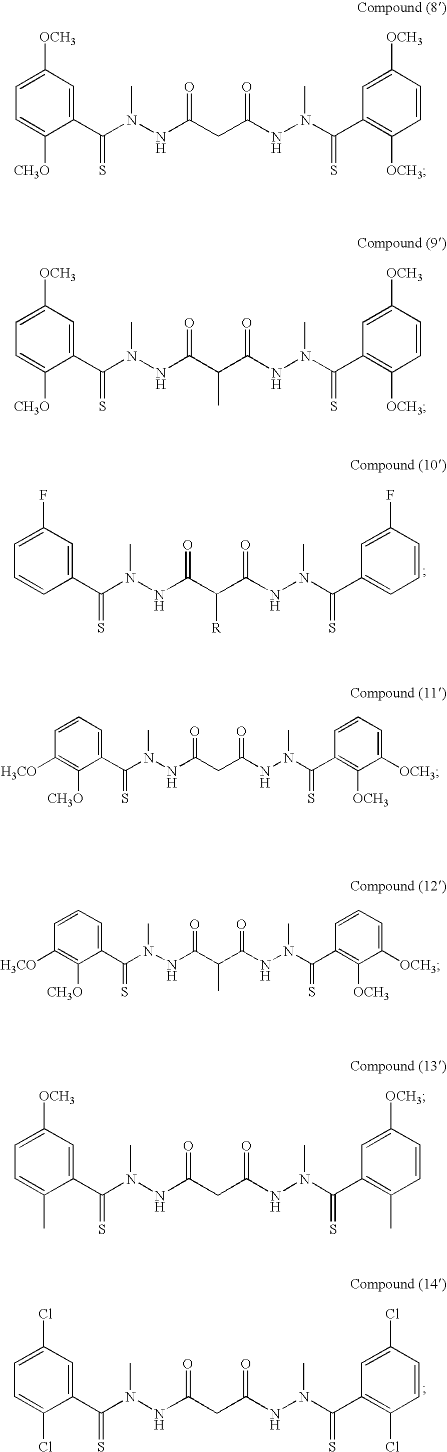

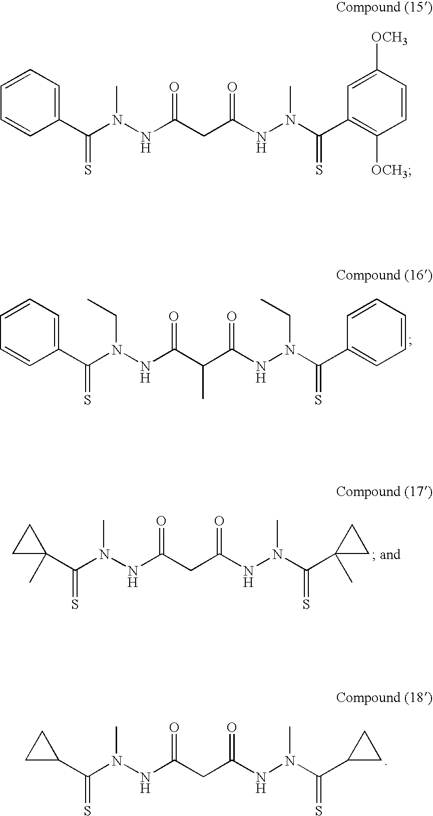

Also included are pharmaceutically-acceptable salts of the bis(thio-hydrazide amides) described herein. These bis(thio-hydrazide amides) can have one or more sufficiently acidic protons that can react with a suitable organic or inorganic base to form a base addition salt. Base addition salts include those derived from inorganic bases, such as ammonium or alkali or alkaline earth metal hydroxides, carbonates, bicarbonates, and the like, and organic bases, such as alkoxides, alkyl amides, alkyl and aryl amines, and the like. Such bases useful in preparing the salts of this invention thus include sodium hydroxide, potassium hydroxide, ammonium hydroxide, potassium carbonate, and the like.

For example, pharmaceutically-acceptable salts of the bis(thio-hydrazide amides) (e.g., those represented by Structural Formulas I-V or Compounds (1′)-(18′)) are those formed by the reaction of the bis(thio-hydrazide amide) with one equivalent of a suitable base to form a monovalent salt (i.e., the compound has single negative charge that is balanced by a pharmaceutically-acceptable counter cation, e.g., a monovalent cation) or with two equivalents of a suitable base to form a divalent salt (e.g., the compound has a two-electron negative charge that is balanced by two pharmaceutically-acceptable counter cations, e.g., two pharmaceutically-acceptable monovalent cations or a single pharmaceutically-acceptable divalent cation). Divalent salts of the bis(thio-hydrazide amides) are preferred. “Pharmaceutically acceptable” means that the cation is suitable for administration to a subject. Examples include Li⁺, Na⁺, K⁺, Mg²⁺, Ca²⁺ and NR₄⁺, wherein each R is independently hydrogen, an optionally substituted aliphatic group (e.g., a hydroxyalkyl group, aminoalkyl group or ammoniumalkyl group) or optionally substituted aryl group, or two R groups, taken together, form an optionally substituted non-aromatic heterocyclic ring optionally fused to an aromatic ring. Generally, the pharmaceutically-acceptable cation is Li⁺, Na⁺, K⁺, NH₃(C₂H₅OH)⁺ or N(CH₃)₃(C₂H₅OH)⁺, and more typically, the salt is a disodium or dipotassium salt, preferably the disodium salt.

Bis(thio-hydrazide amides) with a sufficiently basic group, such as an amine can react with an organic or inorganic acid to form an acid addition salt. Acids commonly employed to form acid addition salts from compounds with basic groups are inorganic acids, such as hydrochloric acid, hydrobromic acid, hydroiodic acid, sulfuric acid, phosphoric acid, and the like, and organic acids, such as p-toluenesulfonic acid, methanesulfonic acid, oxalic acid, p-bromophenyl-sulfonic acid, carbonic acid, succinic acid, citric acid, benzoic acid, acetic acid, and the like. Examples of such salts include the sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caproate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, sulfonate, xylenesulfonate, phenylacetate, phenylpropionate, phenylbutyrate, citrate, lactate, gamma-hydroxybutyrate, glycolate, tartrate, methanesulfonate, propanesulfonate, naphthalene-1-sulfonate, naphthalene-2-sulfonate, mandelate, and the like.

Salts of the bis(thio-hydrazide amide) compounds described herein can be prepared according to methods described in copending and co-owned Patent Application Ser. No. 60/582,596, filed Jun. 23, 2004. The neutral compounds can be prepared according to methods described in U.S. Pat. Nos. 6,800,660 and 6,762,204, both entitled “Synthesis of Taxol Enhancers”, and U.S. Publication No. 20030069225 entitled “Synthesis of Taxol Enhancers”, and also according to methods described in co-pending and co-owned US Published Application No. 20040225016. The entire teachings of each document referred to in this application is expressly incorporated herein by reference.

As used herein, Compound 1 refers to the following structure:

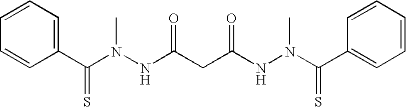

As used herein, Compound 2 refers to the following structure:

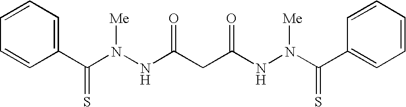

As used herein, Compound 3 refers to the following structure:

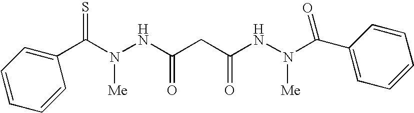

As used herein, Compound 4 refers to the following structure:

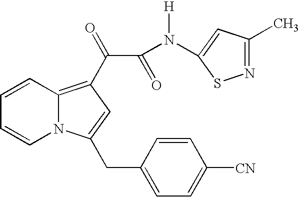

As described, the compounds of the invention exhibit one or more of a subset of properties. In particular embodiments, the compounds are able to disrupt cytoskeletal structure and/or cell morphology when combined with a cell. In one embodiment, the compound can disrupt organization of the actin cytoskeleton of a cell. For example, as described herein (see, e.g., Example 4), treatment of cells with Compound 1, resulted in dramatic alterations in the cells' actin cytoskeletal networks. Specifically, Compound 1 treatment resulted in the disappearance of cytosolic actin bundles (parallel actin fibers), resulting in a more prominent cortical actin network (the area just below the cell membrane that contains the actin network) (FIG. 22).

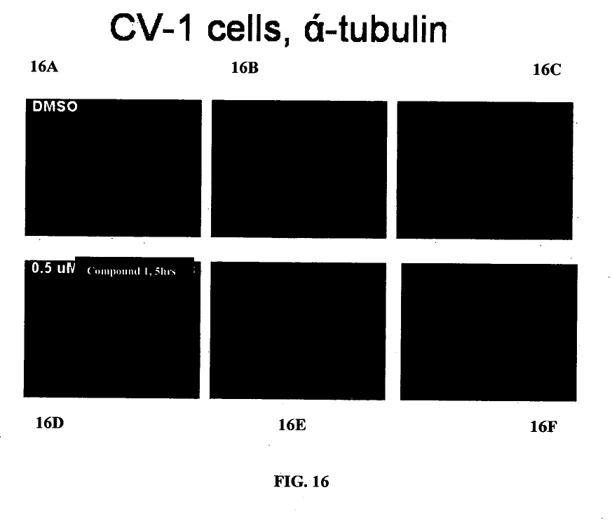

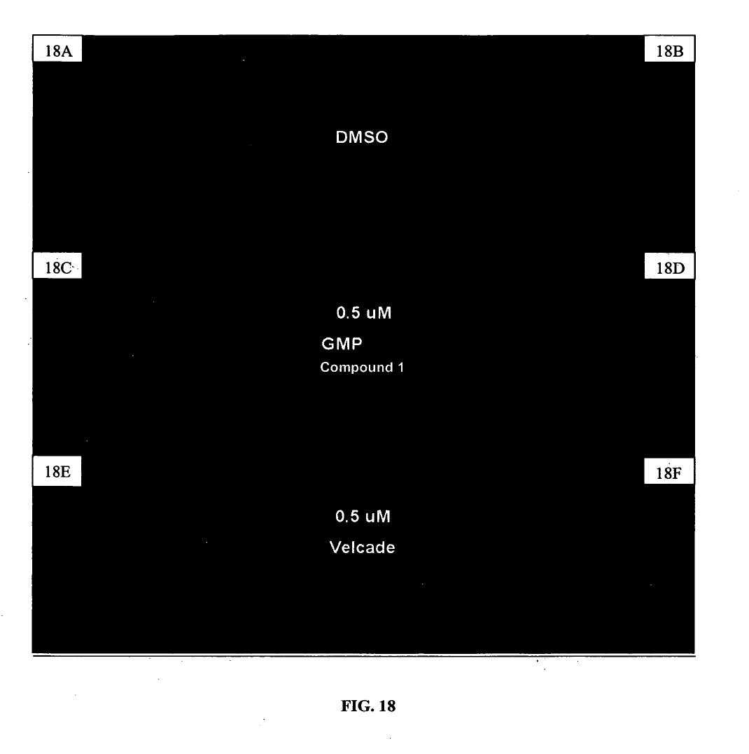

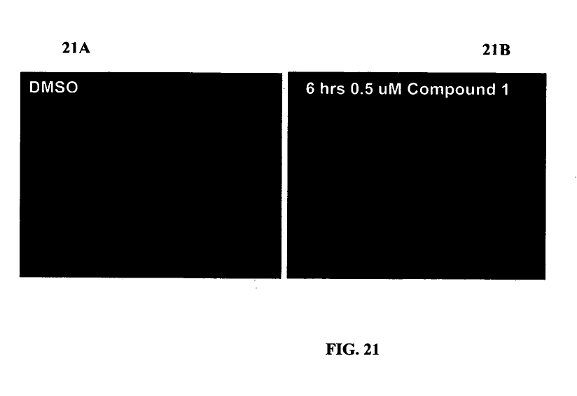

In other embodiments, the compounds of the invention can disrupt organization of the microtubule network of a cell. For example, as described herein (see, e.g., Example 4), treatment of cells with Compound 1, resulted in dramatic alterations in the cells' microtubule network. Specifically, Compound 1 treatment resulted in loss of centrosomal enucleated microtubules, clearing up of microtubules from the cytosol, and coiling up of microtubules around the nucleus of the cell periphery (FIGS. 18 and 21). Compound 1 treatment resulted in an uneven distribution of microtubules throughout the cytoplasm, with a dense microtubule network around the perinuclear region but a sparse microtubule network at the periphery of the cell (FIGS. 16D-16F).

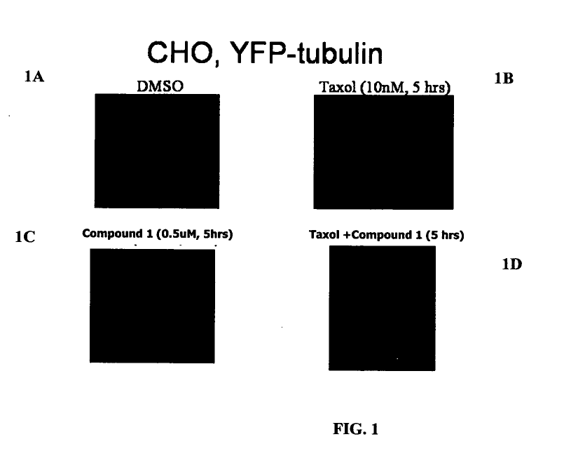

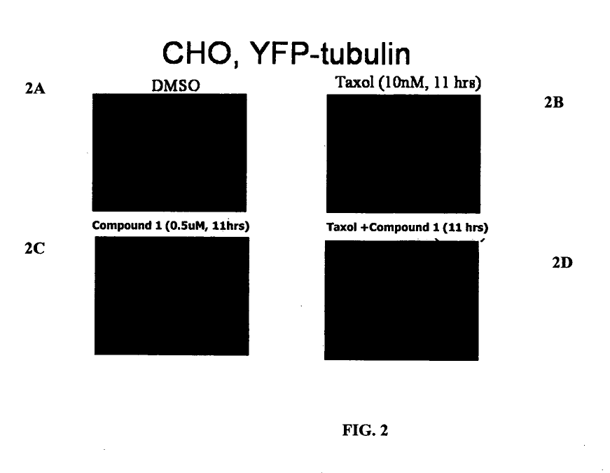

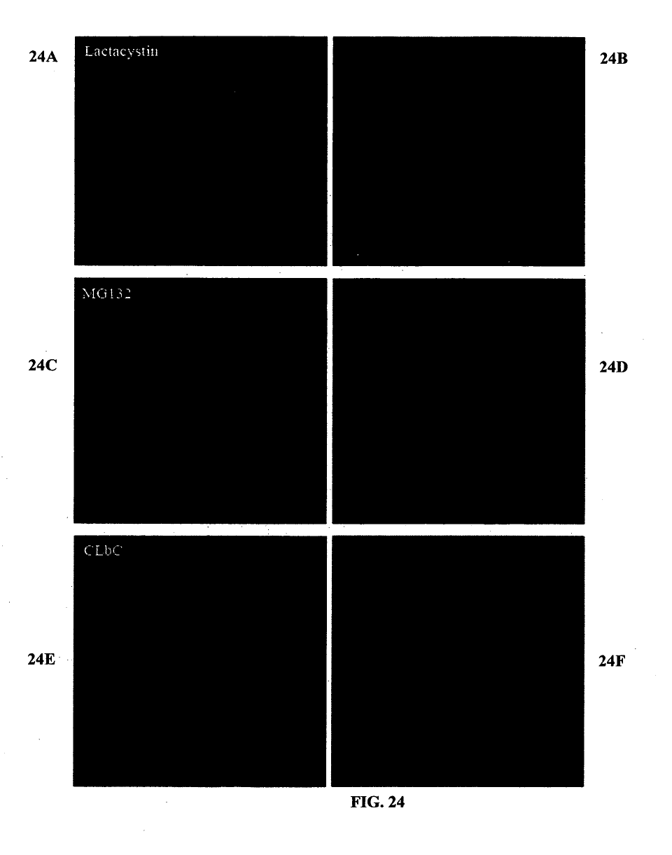

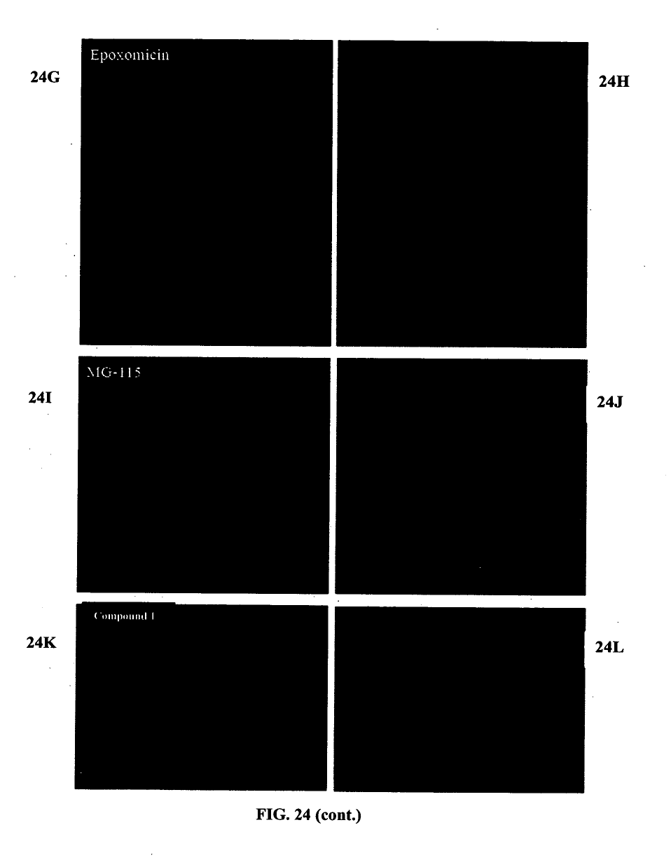

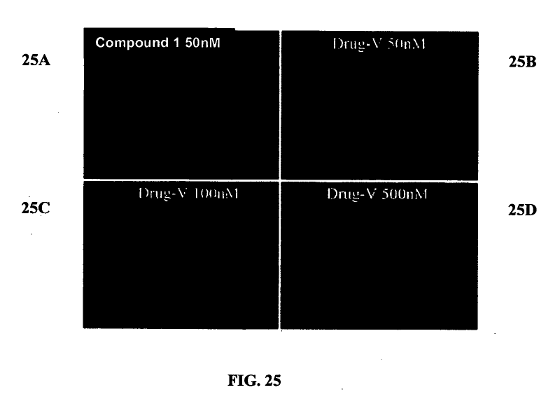

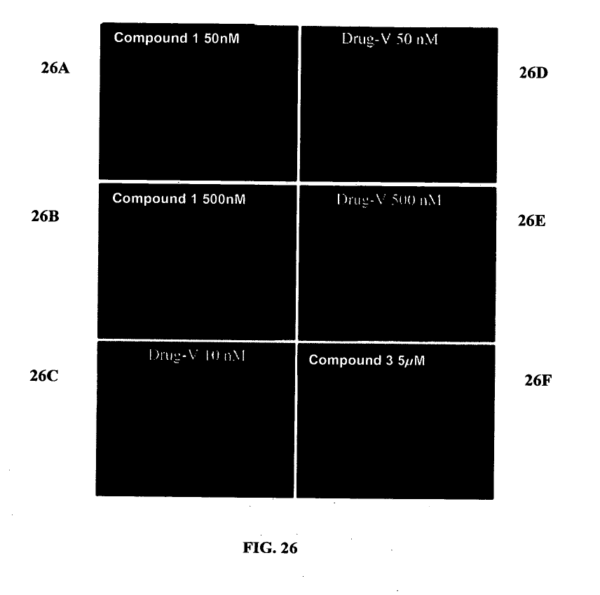

In other embodiments, the compounds of the invention induce accumulation of tubulin at centrosomes but not accumulation of tubulin in the nucleus of a cell. For example, as described herein (see, e.g., Examples 1 and 5), treatment of cells with Compound 1 dramatically affected centrosome structure (FIGS. 1-4). Specifically, Compound 1 treatment resulted in a time-dependent accumulation of tubulin at the centrosomes (FIGS. 1 and 2). In contrast to other known proteasome inhibitors (e.g., ALLN, MG132, Lactacystin, MG115, clasto-Lactacystin β-Lactone (cLβL), Epoxomicin, Velcade), treatment of which resulted in both accumulation of tubulin at the centrosomes and in the nucleus (FIGS. 24A-24E, 25D, 26F and 26E), Compound 1 treatment induced accumulation of tubulin only at the centrosomes (FIGS. 25F, 25A, 26A and 26B). While this embodiment is directed to compounds that result in the accumulation of tubulin (e.g., α-tubulin, β-tubulin, γ-tubulin) at centrosomes but not in the nucleus (centrosomal proteasome inhibitors), the invention encompasses compounds that result in the accumulation of other proteins at the centrosome but not in the nucleus. Suitable proteins include any protein that is expressed both at centrosomes and in the nucleus, wherein the protein is subject to proteasome degradation (and consequently is accumulated when subjected to a proteasome inhibitor). Such proteins include, e.g., Hsp70, Hsp90 and other Hsp members, as well as centrosome-associated proteins including pericentrin, CP140, centrin, tubulin (e.g., gamma-tubulin, alpha-tubulin, beta-tubulin), AKAP450, SKP1p, cyclin-dependent kinase 2-cyclin E (Cdk2-E), kendrin, Protein kinase C-theta, EB1 protein, Nek2, protein kinase A type II isozymes, Hsp70, heat shock Cognate 70 (HSC70), PH33, AIKs, human SCF(SKP2) subunit p19(SKP1), STK15/BTAK, C-Nap1, Tau-like proteins, cyclin E, p53, retinoblastoma protein pRB, BRCA1, dynein and NuMA. Other suitable centrosome associated proteins include, e.g., Cep27 (GenBank Accession No. NP_{—}060567); Cep41 (GenBank Accession No. NP_{—}061188); CepS7 (GenBank Accession No. Q9BVF9); Cep63 (GenBank Accession No. Q9H8N0); Cep68 (GenBank Accession No. NP_{—}055962); Cep70 (GenBank Accession No. NP_{—}077817); Cep72 (GenBank Accession No. Q9P209); Cep 76 (GenBank Accession No. NP_{—}079175); Cep78 (GenBank Accession No. Q9H9N3); Cep131 (GenBank Accession No. Q9UPN4); Cep135 (GenBank Accession No. NP_{—}055460); Cep 152 (GenBank Accession No. O94986); Cep 164 (GenBank Accession No. NP_{—}055771); Cep192 (GenBank Accession No. NP_{—}115518); Cep215 (GenBank Accession No. NP_{—}060719); Cep 290 (GenBank Accession No. O15078); ALMS1 (GenBank Accession No. NP_{—}055935; OFD-1 (GenBank Accession No. O75665); NA-14 (GenBank Accession No. O43805); CCCAP (GenBank Accession No. O60527); CP100 (GenBank Accession No. O43303); Rootletin (GenBank Accession No. NP_{—}055490); FOP (GenBank Accession No. NP_{—}008976). See Andersen, J. S., et al., Nature 426:570-574 (2003; the entire teachings of which are incorporated herein by reference. Suitable candidate centrosome associated proteins include, e.g., CAP350 (GenBank Accession No. NP_{—}055625); KIAA1731 (UniProt KB/TrEMBL Accession No. Q9C0D2); KIAA1074 (GenBank Accession No. NP_{—}055703); KARP-1-binding protein (GenBank Accession No. NP_{—}055627); Golgin-160 (GenBank Accession No. NP_{—}005886); KIAA0542 (UniProt KB/TrEMBL Accession No. Q8WU14); FLJ31872 (GenBank Accession No. NP_{—}663622); FLJ00020 (UniProt KB/TrEMBL Accession No. Q9H7P7); KIAA1764 (UniProt KB/TrEMBL Accession No. Q96DK7); Ubiquitin-activating enzyme E1 (SWISS-PROT Accession No. P22314); NGAP-like protein (UniProt KB/TrEMBL Accession No. Q96SE1); Autoantigen (UniProt KB/TrEMBL Accession No. Q13025); Lyst-interacting protein LIP8 (UniProt KB/TrEMBL Accession No. Q8N137); AY099107 (GenBank Accession No. NP_{—}653319); FLJ38327 (UniProt KB/TrEMBL Accession No. Q8NDE8); FLJ12907 (UniProt KB/TrEMBL Accession No. Q9HCJ8); Progesterone-induced blocking factor 1 (UniProt KB/TrEMBL Accession No. Q8WXW3); FLJ30655 (UniProt KB/TrEMBL Accession No. Q96NL6); Mdn1 (UniProt KB/TrEMBL Accession No. Q8TC05); Kinesin-like protein KIF2 (SWISS-PROT Accession No. O00139); MGC20806 (GenBank Accession No. NP_{—}659436); KIAA0841 (UniProt KB/TrEMBL Accession No. O94927); NEDD1 (UniProt KB/TrEMBL Accession No. Q8NA30); Unconventional myosin 1G methonine form (UniProt KB/TrEMBL Accession No. Q96RI5); IT1 (UniProt KB/TrEMBL Accession No. 043606); FEZ1 (GenBank Accession No. NP_{—}066300); FLJ35779 (UniProt KB/TrEMBL Accession No. Q8NA72); FLJ14640 (GenBank Accession No. NP_{—}116205); DKFZp761A078 (UniProt KB/TrEMBL Accession No. Q8N3K0); TUWD12 (GenBank Accession No. NP_{—}758440); BC282485_{—}1 (UniProt KB/TrEMBL Accession No. Q9Y6R₉); FLJ13215 (GenBank Accession No. NP_{—}079280); WD-repeat protein 8 (SWISS-PROT Accession No. Q9P2S5); FLJ10565 (GenBank Accession No. NP_{—}060610); FLJ90366 (UniProt KB/TrEMBL Accession No. Q8NCB8); FLJ90808 (GenBank Accession No. NP_{—}056241); FLJ32194 (UniProt KB/TrEMBL Accession No. Q9NS50); C14orf60 (GenBank Accession No. NP_{—}803546); Nucleoside diphosphate kinase 7 (SWISS-PROT Accession No. Q9Y5B8); FLJ22363 (GenBank Accession No. NP_{—}060285); FLJ23047 (GenBank Accession No. NP_{—}078824). See Andersen, J. S., et al., Nature 426:570-574 (2003); the entire teachings of which are incorporated herein by reference. Without wishing to be bound to any theory, it is thought that the compounds of the invention act indirectly and/or require a cellular cofactor(s) for their activities (e.g., the activities described herein (e.g., proteasome inhibitory activity)).

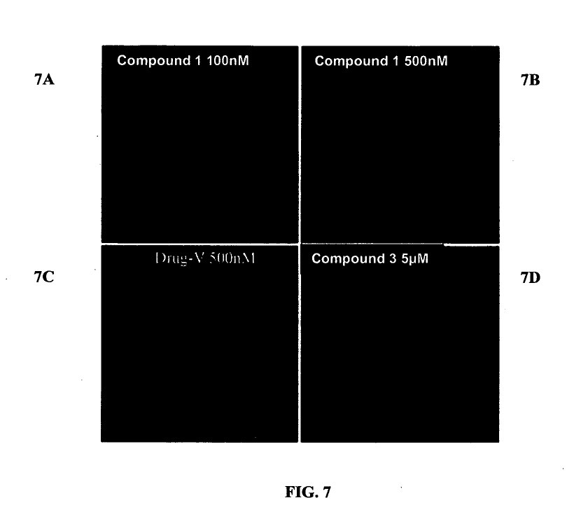

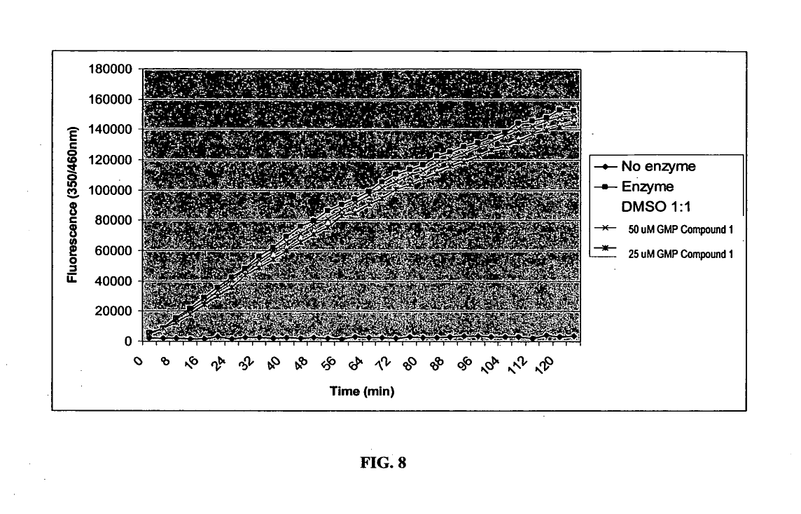

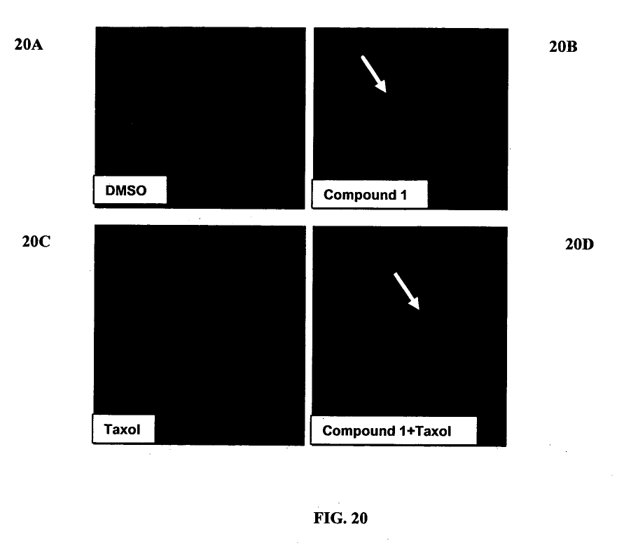

In other embodiments, the compounds of the invention induce accumulation of tubulin at centrosomes at a concentration of 500 nM or less within four hours. For example, as described herein, after 4 hours of treatment of cells with 100 nM Compound 1, accumulation of tubulin at centrosomes could be observed (FIG. 7A, arrows). At a lower concentration (50 nM), Compound 1 induced accumulation of tubulin at centrosomes within 8 hours (FIG. 25A). In contrast, induction of accumulation of tubulin at centrosomes by treatment with the stronger proteasome inhibitor, Velcade, required a higher concentration (500 nM; FIG. 25D) and/or a longer time period (8 hours (FIG. 25D) or 20 hours (FIGS. 26D, 26E)). Moreover, as described herein, in addition to centrosomal and perinuclear accumulation, Velcade (and other known inhibitors) induced accumulation of tubulin in the nucleus.

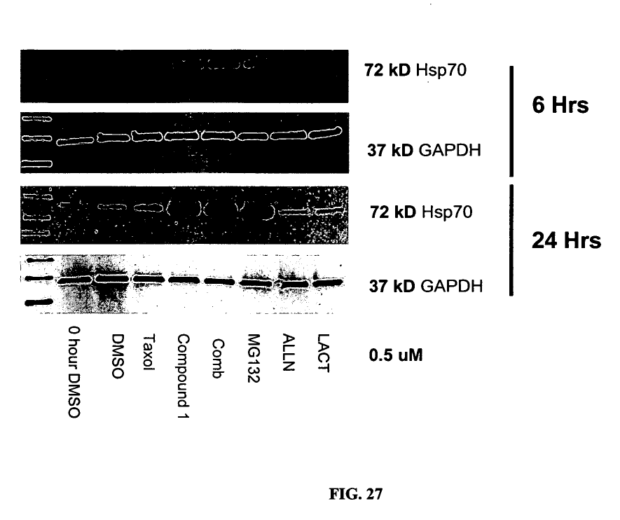

In other embodiments, the compounds of the invention induce accumulation of Hsp70 but only possesses weak-to-moderate proteasome inhibitory activity. As described herein, only a subset of proteasome inhibitors induced accumulation of Hsp70 when administered to cells (Example 6). Administration of Compound 1 strongly induced Hsp70 expression after 6 hours and 24 hours of treatment (FIG. 27). MG132 also induced Hsp70 expression after 6 hours and 24 hours of treatment, but was not as potent an inducer as Compound 1 (FIG. 27). The same concentration of ALLN was unable to induce expression of Hsp70.

Specifically, relative to Hsp70 expression in a DMSO control sample, the percentage change in Hsp70 expression after 6 hours of treatment was as follows: 0.5 μM Taxol (86%); 0.5 μM Compound 1 (980%); 0.5 μM Taxol+Compound 1 (1203%); 0.5 μM MG132 (472%); and 0.5 μM ALLN (82%). The percentage change in Hsp70 expression after 24 hours of treatment, relative to Hsp70 expression in a DMSO control sample, was as follows: 0.5 μM Taxol (556%); 0.5 μM Compound 1 (2121%); 0.5 μM Taxol+Compound 1 (2974%); 0.5 μM MG132 (3016%); and 0.5 μM ALLN (137%). Calculations of the percentage changes were made by using the automated band analysis tools of the Kodak 1D (v.3.6.3) program. Band intensities were normalized to background and GAPDH.

As used herein, “Hsp70” includes each member of the family of heat shock proteins having a mass of about 70-kilodaltons, including forms such as constitutive, cognate, cell-specific, glucose-regulated, inducible, etc. Examples of specific Hsp70 proteins include hsp70, hsp70hom, hsc70, Grp78/BiP, mt-hsp70/Grp75, and the like. In one embodiment, the compounds of the invention induce accumulation of inducible Hsp70. Functionally, the 70-kDa Hsp (Hsp70) family is a group of chaperones that assist in the folding, transport, and assembly of proteins in the cytoplasm, mitochondria, and endoplasmic reticulum. In humans, the Hsp70 family encompasses at least 11 genes encoding a group of highly related proteins. See, e.g., Tavaria et al., Cell Stress Chaperones 1(1):23-28 (1996); Todryk et al., Immunology 110(1):1-9 (2003); and Georgopoulos and Welch, Ann. Rev. Cell Biol. 9:601-634 (1993); the entire teachings of these documents are incorporated herein by reference. See also U.S. Provisional Application No. 60/629,595, entitled “Bis(Thio-Hydrazide Amides) For Increasing Hsp70 Expression,” by James Barsoum, Attorney Docket No. 3211.1017-000, filed on Nov. 19, 2004, the entire teachings of which are incorporated herein by reference.

As is known, heat shock proteins (Hsp's) are a group of proteins that are induced in response to cellular stress. These proteins function as chaperones in the proper folding of proteins under normal conditions and especially under extreme stress conditions, such as heat shock, oxidative stress, infection and exposure to toxins. Hsp's therefore play an important role in protein function by maintaining stability and activity, and by preventing inappropriate protein aggregation. It is believed that Hsp's have a role in the inflammatory response and that expression of Hsp's on the surface of cells is important in targeting cytotoxic cells. Hsp's are also thought to play a role in antigen-presentation.

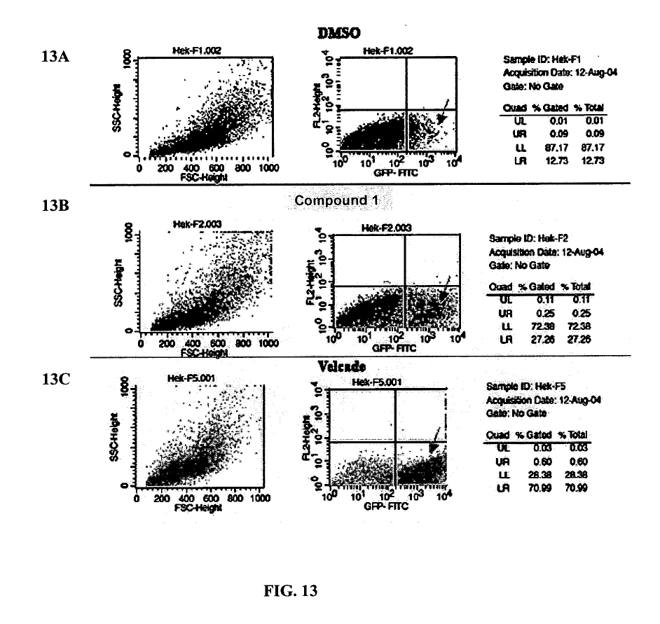

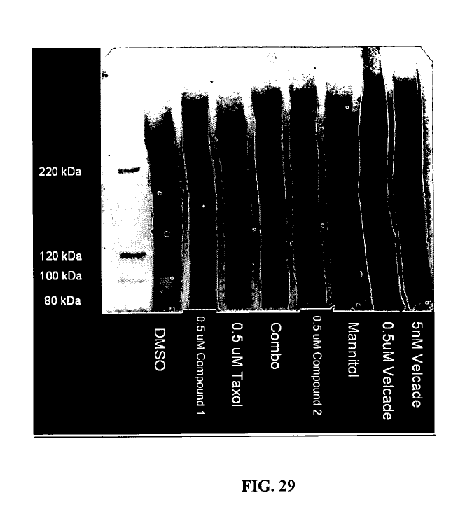

In the above-described embodiments, the compounds of the invention induce accumulation of Hsp70 but only possesses weak-to-moderate proteasome inhibitory activity. As described herein (see, e.g., Example 3), Compound 1 has moderate in vivo proteasome activity, as compared to Velcade. Using a cell-based assay to measure proteasome inhibitory activity in living cells (proteasome-sensor cells), it was observed that a concentration of Compound 1 that was four-fold higher than that of Velcade had a four-fold lower proteasome-inhibitory activity (as measured using a GFP-based proteasome substrate and FACS analysis) (FIG. 13). Three cell-based quantification assays revealed that Compound 1 showed a weak-to-moderate proteasome inhibitory effect in live cells. The first measurement was determined by the increase of proteasome-sensor positive cells treated with Compound 1 or Velcade, as compared to DMSO, by using a cell line expressing the proteasome-sensor protein and flow cytometry analysis. 500 nM Compound 1 and 100 nM Velcade caused increase of the fluorescent positive cells by 14.53% and 58.26%, respectively. Therefore the relative proteasome inhibitory activities are Compound 1:Velcade≈1:20. The second measurement was determined by the increase of strong fluorescent α-tubulin-YFP cells treated with Compound 1 or 50 nM Velcade compared to equivalent amount of DMSO by using a cell line expressing α-tubulin-YFP and flow cytometry analysis. 500 nM Compound 1 and 50 nM Velcade caused increase of the strong-fluorescent positive cells by 20.37% and 27.74%, respectively. Therefore the relative proteasome inhibitory activities are Compound 1:Velcade≈1:13.6. The third measurement was determined by the increase of ubiquitinated proteins in cells treated with Compound 1 and MG-132 compared to equivalent amount of DMSO by using a cell line expressing α-tubulin-YFP and flow cytometry analysis. 500 nM Compound 1 and 500 nM MG-132 caused an increase of ubiquitinated proteins of the cell lysates by values of 210.03 and 212.58, respectively, at 6 hours of treatment in cell culture, and, by values of 243.11 and 357.82 at 24 hours of treatment in cell culture, respectively. Therefore the relative proteasome inhibitory activities are Compound 1:MG-1 32≈1:1 (6 hour treatment) and Compound 1:MG-132≈1:1.5 (24 hour treatment). Using Western blot analysis, Compound 1 showed significantly less accumulation of ubiquitinated proteins in cultured cells than Velcade at the same concentration (500 nM) (FIG. 29). It is known that Compound 1 has very low toxicity in vivo, which is advantageous over other proteasome inhibitors (e.g., Velcade), which have greater toxicity. Without wishing to be bound to any one theory, the decreased toxicity associated with the compounds of the invention may be a result of their strong induction of Hsp70 and moderate proteasome inhibitory activity.

In particular embodiments, the compounds of the invention have a proteasome inhibiting activity that is equal to, or less than, 1/20, 1/10, ⅕, ¼, and/or ½, that of Velcade.

In other embodiments, the compounds of the invention do not have proteasome inhibitory activity when assayed on purified proteasomes. As described herein, Compound 1 did not inhibit the activity of isolated proteasomes when tested using an in vitro assay (Example 2). Specifically, when assayed using the Calbiochem 20S Proteasome Assay Kit (Calbiochem, San Diego, Calif.), which measures the degradation of a fluorogenic substrate, even a 50 μM concentration of Compound 1 did not exhibit significant proteasome inhibitory activity (FIGS. 8 and 9). In contrast, Velcade achieved almost complete inhibition in this assay even at a concentration of 5 nM (FIG. 9). Similarly, other proteasome inhibitors (e.g., ALLN, Lactacystin, MG132) that were tested also exhibited proteasome inhibitory activity when assayed using this in vitro assay (FIGS. 8, 9 and 38). As described, without wishing to be bound to any theory, it is thought that the compounds of the invention act indirectly and/or require a cellular cofactor(s) for their proteasome inhibitory activity.

The compounds of the invention comprise one or more of a subset of properties (e.g., disruption of organization of an actin cytoskeleton of a cell, disruption of organization of a microtubule network of a cell, induction of accumulation of tubulin at centrosomes but not the nucleus of a cell, induction of accumulation of tubulin at centrosomes at a concentration of 500 nM or less within four hours, induction of accumulation of Hsp70 and weak-to-moderate proteasome inhibitory activity, no proteasome inhibitory activity when assayed on purified proteasomes). In one embodiment, the compounds have all of these properties. In other embodiments, the compounds have any 1, 2, 3, 4 or 5, of these properties. For example, the compound may disrupt organization of both the actin cytoskeleton and microtubule network of a cell. In one embodiment, the compound induces accumulation of tubulin at centrosomes but not in the nucleus of a cell and optionally also disrupts organization of both the actin cytoskeleton and microtubule network of a cell. Compounds comprising other combinations of the afore-mentioned properties are also encompassed by the invention.

Methods of Disrupting Centrosome Activity and Methods of Treatment

In certain embodiments, the invention is a method of disrupting centrosome activity in a subject in need thereof comprising administering an effective amount of a compound of the invention (e.g., a compound comprising one or more of the described properties, with the proviso that the compound is not a compound represented by Structural Formula (I)).

In these embodiments, the method comprises administering a compound of the invention to a subject in need thereof, wherein the subject has one or more conditions for which the use of a centrosome disrupter is known to be beneficial. Suitable conditions include, but are not limited to, cancer and non-cancerous proliferative conditions, conditions marked by excessive or accelerated protein degradation, Hsp70-responsive disorders and cystic fibrosis, among others.

In one embodiment, the method comprises administering a compound of the invention to a subject with cancer. Cancers that can be treated or prevented by this method include, but are not limited to, human sarcomas and carcinomas, e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma, leukemias, e.g., acute lymphocytic leukemia and acute myelocytic leukemia (myeloblastic, promyelocytic, myelomonocytic, monocytic and erythroleukemia), chronic leukemia (chronic myelocytic (granulocytic) leukemia and chronic lymphocytic leukemia), and polycythemia vera, lymphoma (Hodgkin's disease and non-Hodgkin's disease), multiple myeloma, Waldenstrobm's macroglobulinemia, and heavy chain disease.

Other examples of leukemias include acute and/or chronic leukemias, e.g., lymphocytic leukemia (e.g., as exemplified by the p388 (murine) cell line), large granular lymphocytic leukemia, and lymphoblastic leukemia, T-cell leukemias, e.g., T-cell leukemia (e.g., as exemplified by the CEM, Jurkat, and HSB-2 (acute), YAC-1 (murine) cell lines), T-lymphocytic leukemia, and T-lymphoblastic leukemia, B cell leukemia (e.g., as exemplified by the SB (acute) cell line), and B-lymphocytic leukemia, mixed cell leukemias, e.g., B and T cell leukemia and B and T lymphocytic leukemia, myeloid leukemias, e.g., granulocytic leukemia, myelocytic leukemia (e.g., as exemplified by the HL-60 (promyelocyte) cell line), and myelogenous leukemia (e.g., as exemplified by the K562 (chronic) cell line), neutrophilic leukemia, eosinophilic leukemia, monocytic leukemia (e.g., as exemplified by the THP-1 (acute) cell line), myelomonocytic leukemia, Naegeli-type myeloid leukemia, and nonlymphocytic leukemia. Other examples of leukemias are described in Chapter 60 of The Chemotherapy Sourcebook, Michael C. Perry Ed., Williams & Williams (1992) and Section 36 of Holland Frie Cancer Medicine 5th Ed., Bast et al. Eds., B. C. Decker Inc. (2000). The entire teachings of the preceding references are incorporated herein by reference.

In this method, the compounds of the invention may also be used in therapies directed to proliferative conditions other than cancer. Examples of non-cancerous proliferative disorders include, but are not limited to, smooth muscle cell proliferation, systemic sclerosis, cirrhosis of the liver, adult respiratory distress syndrome, idiopathic cardiomyopathy, lupus erythematosus, retinopathy, e.g., diabetic retinopathy or other retinopathies, cardiac hyperplasia, reproductive system-associated disorders, such as benign prostatic hyperplasia and ovarian cysts, pulmonary fibrosis, endometriosis, fibromatosis, harmatomas, lymphangiomatosis, sarcoidosis, desmoid tumors and the like.

Smooth muscle cell proliferation includes proliferative vascular disorders, for example, intimal smooth muscle cell hyperplasia, restenosis and vascular occlusion, particularly stenosis following biologically- or mechanically-mediated vascular injury, e.g., vascular injury associated with balloon angioplasty or vascular stenosis. Moreover, intimal smooth muscle cell hyperplasia can include hyperplasia in smooth muscle other than the vasculature, e.g., hyperplasia in bile duct blockage, in bronchial airways of the lung in asthma patients, in the kidneys of patients with renal interstitial fibrosis, and the like.

Non-cancerous proliferative disorders also include hyperproli-feration of cells in the skin, such as psoriasis and its varied clinical forms, Reiter's syndrome, pityriasis rubra pilaris, and hyperproliferative variants of disorders of keratinization (e.g., actinic keratosis, senile keratosis), scleroderma, and the like.

Some proteasome inhibitors are known to induce heat shock proteins, particularly Hsp70. Increased expression of heat shock proteins in the Hsp70 family are known to protect a broad range of cells from adverse effects associated with a variety of cellular stresses. As such, the compounds of the invention are suitable for treating Hsp70-responsive disorders. Specific examples of Hsp70-responsive conditions include, but are not limited to, Alzheimer's disease, Huntington's disease, spinal/bulbar muscular atrophy and other neuromuscular atrophies, familial amyotrophic lateral sclerosis, ischemia, seizure, hypothermia, hyperthermia, burn trauma, atherosclerosis, radiation exposure, glaucoma, toxin exposure, mechanical injury, inflammation, autoimmune disease, infection (bacterial, viral, fungal, or parasitic), and the like. See, e.g., U.S. Provisional Application No. 60/629,595, entitled “Bis(Thio-Hydrazide Amides) For Increasing Hsp70 Expression,” by James Barsoum, Attorney Docket No. 3211.1017-000, filed on Nov. 19, 2004.

Other conditions known to respond positively to treatment using proteasome inhibitors include, e.g., cystic fibrosis (see, e.g., U.S. Pat. No. 6,723,703), conditions marked by excessive or accelerated protein degradation, such as muscle-wasting and low muscle mass diseases (see, e.g., U.S. Pat. Nos. 5,972,636, 5,340,736, 5,565,351, Debigare R. and S. R. Price, Am. J. Physiol. Renal Physiol. 285: F1-F8 (2003)), skeletal system disorders resulting from bone loss or low bone density (see, e.g., U.S. Pat. Nos. 6,462,019 and 6,656,904), conditions related to hair growth (see, e.g., U.S. Pat. Nos. 6,410,512 and 6,656,904), and dry-eye disorders (see, e.g., U.S. Pat. No. 6,740,674). The entire teachings of the preceding references are incorporated herein by reference.

Muscle-wasting conditions include those resulting from cachexia, muscle disuse (atrophy) and denervation, nerve injury, fasting, chronic renal failure, and the like (see, e.g., U.S. Pat. Nos. 5,972,636, 5,340,736, 5,565,351, Debigare R. and S. R. Price, Am. J. Physiol. Renal Physiol. 285: F1-F8 (2003)). Also included are conditions resulting in low muscle mass due to catabolic states, such as uremia, diabetes, sepsis, metabolic acidosis and cancer (Debigare R. and S. R. Price, Am. J. Physiol. Renal Physiol. 285: F1-F8 (2003)).

Conditions related to skeletal system disorders resulting from bone loss or low bone density include, but are not limited to, repair of bone defects and deficiencies, such as those occurring in closed, open, and non-union fractures, prophylactic use in closed and open fracture reduction, promotion of bone healing in plastic surgery, stimulation of bone ingrowth into non-cemented prosthetic joints and dental implants, elevation of peak bone mass in pre-menopausal women, treatment of growth deficiencies, treatment of periodontal disease and defects, and other tooth repair processes, increase in bone formation during distraction osteogenesis, age-related osteoporosis, post-menopausal osteoporosis, glucocorticoid-induced osteoporosis or disuse osteoporosis, arthritis, or any condition that benefits from stimulation of bone formation (see, e.g., U.S. Pat. No. 6,656,904). Proteasome inhibitors can also be useful in repair of congenital, trauma-induced or surgical resection of bone, and in cosmetic surgery. Further, these compounds can be used for limiting or treating cartilage defects or disorders, and may be useful in wound healing or tissue repair (see, e.g., U.S. Pat. Nos. 6,462,019 and 6,656,904).

Conditions related to the stimulation of hair growth include, but are not limited to, male pattern baldness, alopecia caused by chemotherapy, hair thinning resulting from aging, genetic disorders that result in deficiency of hair coverage, and, in animals, providing additional protection from cold temperatures (see, e.g., U.S. Pat. Nos. 6,410,512 and 6,656,904).

Dry-eye disorders may result from excessive inflammation in relevant ocular tissues, such as the lacrimal and meibomian glands, and include conditions requiring wetting of the eye, including symptoms of dry eye associated with refractive surgery such as LASIK surgery (see, e.g., U.S. Pat. No. 6,740,674).

In other embodiments, the invention is a method for treating a condition in a subject comprising administering an effective amount of a compound of the invention. In these embodiments, the compound comprises one or more of the described properties (e.g., disruption of organization of an actin cytoskeleton of a cell, disruption of organization of a microtubule network of a cell, induction of accumulation of tubulin at centrosomes but not the nucleus of a cell, induction of accumulation of tubulin at centrosomes at a concentration of 500 nM or less within four hours, induction of accumulation of Hsp70 and weak-to-moderate proteasome inhibitory activity, no proteasome inhibitory activity when assayed on purified proteasomes).

Suitable conditions include those conditions described herein. In one embodiment, the subject's condition is selected from the group consisting of muscle-wasting diseases (e.g., fever, muscle disuse (atrophy) and denervation, nerve injury, fasting, renal failure associated with acidosis, hepatic failure, uremia, diabetes, and sepsis), skeletal system disorders resulting from bone loss or low bone density (e.g., closed fractures, open fractures, non-union fractures, age-related osteoporosis, post-menopausal osteoporosis, glucocorticoid-induced osteoporosis, disuse osteoporosis, arthritis), growth deficiencies (e.g., periodontal disease and defects, cartilage defects or disorders), disorders of hair growth (e.g., male pattern baldness, alopecia caused by chemotherapy, hair thinning resulting from aging, genetic disorders resulting in deficiency of hair coverage), dry-eye disorders (e.g., excessive inflammation in relevant ocular tissues, such as the lacrimal and meibomian glands, dry eye associated with refractive surgery (e.g., LASIK surgery)) and cystic fibrosis.

In one embodiment, the method for treating a condition in a subject comprises administering an effective amount of a compound, wherein the compound is a compound represented by Structural Formula (I). In a particular embodiment, the compound is a compound represented by the following structural formula: 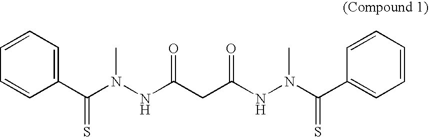
or a pharmaceutically-acceptable salt thereof. In another embodiment, the compound is a disodium or dipotassium salt of the above-depicted structure.

As used herein, the terms “treat”, “treatment” and “treating” refer to administration of one or more therapies (e.g., one or more therapeutic agents, such as the compounds of the invention) to reduce, ameliorate, or prevent the progression, severity and/or duration of a condition (e.g., one or more of the conditions described herein), or to reduce, ameliorate, or prevent one or more symptoms (preferably, one or more discernible symptoms) of a condition. In specific embodiments, the terms “treat”, “treatment” and “treating” refer to the amelioration of at least one measurable physical parameter of a condition, not necessarily discernible by the patient. In other embodiments the terms “treat”, “treatment” and “treating” refer to the inhibition of the progression of a condition, either physically by, e.g., stabilization of a discernible symptom, physiologically by, e.g., stabilization of a physical parameter, or both. In other embodiments the terms “treat”, “treatment” and “treating” refer to the inhibition or reduction in the onset, development or progression of one or more symptoms associated with a condition.

As used herein, the terms “prevent”, “prevention” and “preventing” refer to the prophylactic administration of one or more therapies (e.g., one or more therapeutic agents, such as the compounds of the invention) to reduce the risk of acquiring or developing a condition, or to reduce or inhibit the recurrence, onset or development of one or more symptoms of a particular condition. In a preferred embodiment, a compound of the invention is administered as a preventative measure to a patient, preferably a human, having a genetic or environmental risk factor for a condition.

As used herein, a “subject” is a mammal, preferably a human, but can also be an animal in need of veterinary treatment, e.g., companion animals (e.g., dogs, cats, and the like), farm animals (e.g., cows, sheep, pigs, horses, and the like) and laboratory animals (e.g., rats, mice, guinea pigs, and the like).

As used herein, an “effective amount” is the quantity of compound in which a beneficial clinical outcome is achieved when the compound is administered to a subject. A “beneficial clinical outcome” includes therapeutic or prophylactic treatment of stressed cells via increased activity (e.g., increased proteasome inhibitory activity), resulting in a reduction in the severity of the symptoms associated with a particular condition. The amount of the compound of the invention or composition comprising a compound of the invention, which will be effective in the prevention, treatment, management, and/or amelioration of a particular condition or one or more symptoms thereof, will vary with the nature and severity of the disease or condition, and the route by which the active ingredient is administered. The frequency and dosage will also vary according to factors specific for each patient, e.g., the specific therapy (e.g., therapeutic or prophylactic agents) administered, the severity of the disorder, disease, or condition, the route of administration, as well as age, body weight, response, and the past medical history of the patient. Effective doses may be extrapolated from dose-response curves derived from in vitro or animal model test systems. Suitable regiments can be selected by one skilled in the art by considering such factors and by following, for example, dosages reported in the literature and recommended in Hardman et al., eds., 1996, Goodman & Gilman's The Pharmacological Basis Of Therapeutics 9^{th} Ed, McGraw-Hill, New York; Physician's Desk Reference (PDR) 57^{th} Ed., 2003, Medical Economics Co., Inc., Montvale, N.J., the entire teachings of which are incorporated herein by reference.

Exemplary doses of the compounds of the invention include microgram to milligram amounts of the compound per kilogram of subject or sample weight (e.g., about 1 μg/kg to about 500 mg/kg, about 500 μg/kg to about 250 mg/kg, about 1 mg/kg to about 100 mg/kg, about 10 mg/kg to about 50 mg/kg, and the like).

The compounds described herein can be administered to a subject by any conventional method of drug administration, for example, orally in capsules, suspensions or tablets, or by parenteral administration. Parenteral administration can include, for example, systemic administration, such as by intramuscular, intravenous, subcutaneous, or intraperitoneal injection. The compounds can also be administered orally (e.g., dietary), topically, by inhalation (e.g., intrabronchial, intranasal, oral inhalation or intranasal drops), rectally, vaginally, and the like. In specific embodiments, oral, parenteral, or local administration are preferred modes of administration for treatment of particular conditions.

The compounds described herein can be administered to the subject in conjunction with an acceptable pharmaceutical carrier or diluent as part of a pharmaceutical composition for treatment of a particular condition (e.g., a condition described herein). Formulation of the compound to be administered will vary according to the route of administration selected (e.g., solution, emulsion, capsule, and the like). Suitable pharmaceutically-acceptable carriers may contain inert ingredients which do not unduly inhibit the biological activity of the compounds. The pharmaceutically-acceptable carriers should be biocompatible, i.e., non-toxic, non-inflammatory, non-immunogenic and devoid of other undesired reactions upon administration to a subject. Standard pharmaceutical formulation techniques can be employed, such as those described in Remington's Pharmaceutical Sciences, 16^{th} and 18^{th} eds., Mack Publishing Company, Easton, Pa., 1980-1990. Suitable pharmaceutical carriers for parenteral administration include, for example, sterile water, physiological saline, bacteriostatic saline (saline containing about 0.9% mg/ml benzyl alcohol), phosphate-buffered saline, Hank's solution, Ringer's-lactate and the like. Methods for encapsulating compositions (such as in a coating of hard gelatin or cyclodextran) are known in the art (Baker, et al., “Controlled Release of Biological Active Agents”, John Wiley and Sons, 1986).

In one embodiment, the method comprises topical administration. In such cases, the compounds may be formulated as a solution, gel, lotion, cream or ointment in a pharmaceutically-acceptable form. Actual methods for preparing these, and other, topical pharmaceutical compositions are known or apparent to those skilled in the art and are described in detail in, for example, Remington's Pharmaceutical Sciences, 16^{th} and 18^{th} eds., Mack Publishing Company, Easton, Pa., 1980-1990.

Also included in the present invention are pharmaceutically-acceptable salts of the compounds described herein. For example, as described herein, pharmaceutically-acceptable salts of bis(thio-hydrazide amides) are encompassed by the invention.

In particular embodiments, the invention pertains to use of the compounds described herein for the manufacture of a medicament for the treatment of a condition (e.g., one or more of the conditions described herein).

Methods of Identifying Compounds that Disrupt Centrosome Activity

In one embodiment, the invention is a method for identifying a compound that disrupts centrosome activity comprising combining a cell that expresses a centrosome-associated protein and a test agent; and measuring the accumulation of the centrosome-associated protein at one or more centrosomes of the cell and in a nucleus of the cell. An increase in the accumulation of the centrosome-associated protein at the one or more centrosomes, but no increase in the accumulation of the centrosome-associated protein at the nucleus, relative to a suitable control, indicates that said test agent is a compound that disrupts centrosome activity.

In one embodiment, the method comprises combining a cell that expresses tubulin and a test agent, and measuring the accumulation of tubulin at one or more centrosomes of the cell and/or in a nucleus of the cell. An increase in the accumulation of tubulin at the centrosome(s) and/or nucleus, relative to a suitable control, indicates that the test agent is a proteasome inhibitor.

In one embodiment, the method further comprises assaying the test agent for proteasome inhibitory activity and/or efficacy for treatment of a condition. Suitable assays for measuring proteasome inhibitory activity and/or efficacy for treatment of a condition are known in the art and include, e.g., in vitro and in vivo assays described herein (Examples 2 and 3).

For the methods of the invention, suitable cells include any cell that expresses tubulin (e.g., naturally-occurring cells, appropriate cell lines, recombinant cells). In a particular embodiment, the tubulin-expressing cell is a recombinant cell (e.g., a recombinant cell that expresses exogenous tubulin (e.g., expressed from a sequence of exogenous nucleotides (e.g., a plasmid))). As used herein, a recombinant cell that expresses exogenous tubulin comprises a sequence of exogenous nucleotides (e.g., a plasmid) directing expression of exogenous tubulin. Methods for producing recombinant cells are well known in the art. In one embodiment, the cell that expresses tubulin is selected from the group consisting of a CHO cell, an MCF-7 cell and a CV-1 cell. Other suitable cells for use in the method are known in the art.

In particular embodiments, the methods comprise measuring the accumulation of tubulin at one or more centrosomes of the cell and/or in a nucleus of the cell. Methods for measuring the accumulation of tubulin at a particular location (e.g., at one or more centrosomes, in a nucleus) are well known in the art, and include, e.g., immunodetection, detection of labeled tubulin (e.g., as described herein). In a particular embodiment, the tubulin that is measured comprises a label. Expression of exogenous tubulin that comprises a label facilitates its detection and measuring of its accumulation. For example, as described herein, tubulin labeled with yellow fluorescent protein (YFP) facilitated its detection.

Suitable labels for use in the methods of the invention include, e.g., fluorescent labels, radioisotopes, epitope labels, affinity labels, spin labels, enzyme labels, fluorescent labels, chemiluminescent labels and/or other suitable labels that facilitate detection and/or measuring of the tubulin. In a particular embodiment, the tubulin comprises a fluorescent label. Suitable fluorescent labels include, but are not limited to, fluorescein (e.g., fluorescein isothiocyanate (FITC), NHS-fluorescein), rhodamine, coumarin, Texas red (e.g., Texas red sulfonyl chloride), BODIPY fluorophores, Cascade Blue™ fluorophores, Lucifer Yellow fluorophores, phycobiliproteins, (e.g., B-phycoerythrin, R-phycoerythrin) and derivatives of any of the foregoing (see, e.g., Hermanson, G. T., Bioconjugate Techniques, Academic Press, San Diego, Calif. (1996), p. 298-364). In one embodiment, the label is a fluorescent protein (e.g., yellow fluorescent protein, green fluorescent protein).

Suitable radioactive labels that can be used in the methods include, but are not limited to, iodine-131, iodine-125, bismuth-212, yttrium-90, yttrium-88, technetium-99m, copper-67, rhenium-188, rhenium-186, galium-66, galium-67, indium-111, indium-114m, indium-115 and boron-10 (see, e.g., Hermanson, G. T., Bioconjugate Techniques, Academic Press, San Diego, Calif. (1996), p. 365 et seq.).

Suitable enzyme labels that can be used in the methods include, but are not limited to, horseradish peroxidase (HRP), alkaline phosphatase (AP), β-galactosidase (β-gal), glucose oxidase (GO), maltose binding protein (MBP) and glutathione-S-transferase (GST) (see, e.g., Hermanson, G. T., Bioconjugate Techniques, Academic Press, San Diego, Calif. (1996); the entire teachings of which are incorporated herein by reference). Other suitable enzymes, proteins and/or peptides that possess one or more properties that are suitable for detection of tubulin can also be used.

Suitable affinity labels that can be used in the methods include, but are not limited to, biotin, avidin (e.g., streptavidin)), chitin, amylase, glutathione, other peptide affinity labels. The use of affinity labels (as well as the other labels described herein) can facilitate subsequent isolation and purification of the labeled tubulin.

Suitable epitope labels that can be used in the methods include, but are not limited to, hemagglutinin (HA), FLAG epitopes, and other peptide epitopes labels. In one embodiment, the tubulin comprises a solvent soluble dye (e.g., a solvent soluble laser dye, such as an infrared dye or a near infrared dye).

Tubulin can be labeled using a variety of known methods. For example, and as described herein, recombinant technology can be used to express tubulin comprising a label (e.g., a fluorescent label (e.g., yellow fluorescent protein)). Tubulin can also be subject to direct labeling (e.g., attaching a radioactive atom to a functional group of the tubulin) or indirect labeling (e.g., utilizing a bifunctional agent containing a chemical-reactive group for complexing a radioactive metal) (Hermanson, Id.). In a particular embodiment, the label is a detectable moiety that possesses a specifically identifiable physical property that allows it to be distinguished from other molecules that are present in a heterologous mixture.

In particular embodiments, accumulation of tubulin is measured and an increase in the accumulation of tubulin at the centrosome(s) and/or nucleus, relative to a suitable control, indicates that the test agent is a proteasome inhibitor. Suitable controls include, e.g., tubulin-expressing cells that have not been administered the test agent.

The invention also encompasses proteasome inhibitors identified by such a method.

In a particular embodiment, the method identifies centrosomal proteasome inhibitors. As used herein, a centrosomal proteasome inhibitor is a proteasome inhibitor having proteasome inhibiting activity at one or more centrosomes but lacking proteasome inhibiting activity at other locations in the cell (e.g., in the nucleus). In this method, a cell that expresses tubulin is combined with a test agent, and the accumulation of tubulin is measured at one or more centrosomes of the cell and in the nucleus of the cell. An increase in the accumulation of tubulin at the centrosomes, but no increase in the accumulation of tubulin in the nucleus, relative to a suitable control, indicates that the test agent is a centrosomal proteasome inhibitor. In a particular embodiment, the method further comprises assaying the test agent for proteasome inhibition activity.

In another embodiment, the method identifies a nuclear proteasome inhibitor. As used herein, a nuclear proteasome inhibitor is a proteasome inhibitor having proteasome inhibiting activity in the nucleus of a cell but lacking proteasome inhibiting activity at other locations in the cell (e.g., at the centrosomes). In this method, a cell that expresses tubulin is combined with a test agent, and the accumulation of tubulin is measured at one or more centrosomes of the cell and in the nucleus of the cell. An increase in the accumulation of tubulin in the nucleus of the cell, but no increase in the accumulation of tubulin at the centrosome(s), relative to a suitable control, indicates that the test agent is a nuclear proteasome inhibitor. In a particular embodiment, the method further comprises assaying the test agent for proteasome inhibition activity.

Suitable cells that express tubulin, methods for measuring accumulation of tubulin, and controls are described herein and/or are known in the art.

In one embodiment, the method comprises combining a cell that expresses a centrosome-associated protein and a test agent, and measuring the accumulation of the centrosome-associated protein at one or more centrosomes of the cell and/or in a nucleus of the cell. An increase in the accumulation of the centrosome-associated protein at the centrosome(s) and/or nucleus, relative to a suitable control, indicates that the test agent is a proteasome inhibitor. In a particular embodiment, the method further comprises assaying the test agent for proteasome inhibition activity. Such a method can be used to identify general proteasome inhibitors, as well as centrosomal and nuclear proteasome inhibitors.

Suitable centrosome-associated proteins for use in the methods of the invention include, e.g., pericentrin, CP140, centrin, tubulin (e.g., gamma-tubulin, alpha-tubulin, beta-tubulin), Hsp70, AKAP450, SKP1p, cyclin-dependent kinase 2-cyclin E (Cdk2-E), kendrin, Protein kinase C-theta, EB1 protein, Nek2, protein kinase A type II isozymes, heat shock Cognate 70 (HSC70), PH33, AIKs, human SCF(SKP2) subunit p19(SKP1), STK15/BTAK, C-Nap1, Tau-like proteins, cyclin E, p53, retinoblastoma protein pRB, BRCA1, dynein and NuMA. In one embodiment, the centrosome-associated protein is pericentrin. Other suitable centrosome-associated proteins include those described herein.

Centrosome-associated proteins can be detected and measured as described (e.g., by exogenously expressing with a label, through immunodetection (e.g., using an appropriate antibody). Antibodies that react with centrosome-associated proteins are known in the art and their preparation has been described. See, e.g., Doxsey et al., Cell 76:639 (1994), describing the preparation of antibodies to pericentrin; Steams et al., Cell 76:629 (1994), describing the preparation of antibodies to γ-tubulin; and Salisbury et al., Curr. Opin. Cell Biol. 7:39 (1995), describing the preparation of antibodies to centrin. A number of centrosome proteins are described in Schliwa et al., Trends Cell Biol. 3:377 (1993). Procedures for obtaining other antibodies that react with a centrosomal-associated protein can be carried out using a preparation of a non-human centrosomal-associated protein, e.g., murine pericentrin protein. Preparation of an immunizing antigen, and polyclonal and monoclonal antibody production can be performed using any suitable technique. A variety of methods have been described (see e.g., Kohler et al., Nature, 256: 495-497 (1975) and Eur. J. Immunol. 6: 511-519 (1976); Milstein et al., Nature 266: 550-552 (1977); Koprowski et al., U.S. Pat. No. 4,172,124; Harlow, E. and D. Lane, 1988, Antibodies: A Laboratory Manual, (Cold Spring Harbor Laboratory: Cold Spring Harbor, N.Y.); Current Protocols In Molecular Biology, Vol. 2 (Supplement 27, Summer '94), Ausubel, F. M. et al., Eds., (John Wiley & Sons: New York, N.Y.), Chapter 11, (1991)).

Suitable cells that express a centrosome-associated protein and controls (e.g., a cell that expresses a centrosome-associated protein that has not been administered the test agent) are described herein and/or are known in the art.

In another embodiment, the invention relates to a method for stabilizing one or more exogenously-expressed protein(s) in a cell comprising contacting the cell with a compound of the present invention. Suitable cells include any cell that expresses an exogenous protein and are well known in the art. In a particular embodiment, the cell is a recombinant cell. Methods for producing recombinant cells are well known in the art.

In another embodiment, the invention is a method for increasing the efficacy of antigen presentation in a cell comprising contacting the cell with a compound of the invention and an antigenic peptide. As is known, inhibition of the function of one or more components of the MHC class I antigen processing pathway, which involves the 26S proteasome, results in cells deficient in endogenous peptide loading. Contacting a cell with an exogenous antigenic peptide results in loading of empty class I molecules and is an efficient method for producing an antigen-presenting cell having an increased density of antigen (relative to the density of antigen obtained by employing natural MHC class I antigen presentation pathway). See, e.g., U.S. Pat. No. 5,831,068, the entire teachings of which are incorporated herein by reference.

Any antigenic peptide that is naturally presented on the surface of an antigen-presenting cell can be employed in the method. In a particular embodiment, the antigen is a polypeptide that includes a portion of a protein naturally expressed by a pathogen, such as a bacterium or a virus. If desired, the antigen can be a tumor-specific antigen (i.e., an antigen that is preferentially expressed or present in a tumor cell, as compared to a non-tumor cell). An antigen-presenting cell produced with a tumor-specific antigen can be administered to a mammal in a method of treating or preventing cancer (e.g., a malignant tumor, a carcinoma, or a sarcoma) (U.S. Pat. No. 5,831,068).

A variety of cells can be used in the invention. Preferably, the cell is a mammalian cell, such as a human or mouse cell. The cell can be a primary cell, or it can be a cell of an established cell line. Preferably, the cell is one of the following: a T lymphocyte (e.g., a RMA cell), a B lymphocyte, an adherent or non-adherent splenocyte, an adherent or non-adherent peripheral blood mononuclear cell (PBMC), a dendritic cell (e.g., a spleen-derived dendritic cell, a Langerhans'-dendritic cell, a follicular dendritic cell, or a precursor-derived dendritic cell), a macrophage, a thymoma cell (e.g., an EL4 cell), or a fibroblast. If desired, a combination of cells can be used in the invention. For example, the activity of an MHC class I pathway-associated component can be inhibited in a mixture of adherent and non-adherent PBMC (U.S. Pat. No. 5,831,068).

The present invention will now be illustrated by the following Examples, which are not intended to be limiting in any way. The relevant teachings of all publications cited herein that have not explicitly been incorporated herein by reference, are incorporated herein by reference in their entirety.

### EXEMPLIFICATION

### Example 1

### Compound 1 Induces Accumulation of Tubulin at Centrosomes

Materials and Methods

Wild-type Chinese Hamster Ovary cells (WT CHO) cells were maintained in Ham's F-12 medium supplemented with 10% fetal bovine serum (FBS; HyClone, Logan, Utah). Cells of low density (˜20%) growing on 2-well chambered cover-slips (Labtek (Campbell, Calif.) or Fisher Scientific) were transfected with a mammalian expression vector encoding α-tubulin-YFP (Clontech, Palo Alto, Calif.) with the use of FuGENE 6 (Roche Molecular Biochemicals, Indianapolis, Ind.), according to the manufacturer's instructions. Twenty-four hours after transfection, the cells were cultured in 400 μg/ml G418 (Invitrogen, Carlsbad, Calif.)-containing selection medium for 2 weeks. Living cells were examined using a fluorescent microscope for α-tubulin-YFP expression. Cells in single colonies containing microtubules labeled with α-tubulin-YFP were lifted and expanded in G418-containing medium. Expression of α-tubulin-YFP was confirmed by the presence of the tubulin-YFP labeled microtubule pattern identical to immunostained microtubule pattern of non-transfected cells, as well as by subjecting the cells to Western blot analysis using an anti-GFP antibody (Roche Molecular Biochemicals, Basel, Switzerland) and confirming the correct mass of the α-tubulin-YFP chimera protein. Expressed tubulin-YFP was detected as a single band in Western blots. The tubulin-YFP expressing cell lines (referred as CHO-α-tubulin-YFP cells) were used in the studies.

CHO-α-tubulin-YFP cells were cultured in 2-well chambered cover-slips (Labtek (Campbell, Calif.) or Fisher Scientific) 24 hours before treatment. For comparison of the centrosomal effects of treatment with Compound 1, Compound 1+Taxol, and Taxol, CHO-α-tubulin-YFP cells were treated with Compound 1, Compound 1+Taxol, Taxol, or equivalent concentrations of DMSO-containing media for various time periods before imaging. For comparison of the effects of Compound 1 and known proteasome inhibitors on centrosomes, the nucleus, and perinuclear regions, CHO-α-tubulin-YFP cells were treated with Compound 1, ALLN (Calbiochem, San Diego, Calif.), lactacystin (Calbiochem, San Diego, Calif.) or MG132 (Calbiochem, San Diego, Calif.), and imaged at various time points from 3 hours to 24 hours after treatment.

Tubulin-YFP fluorescence in living cells or fixed cells was captured using a standard filter for FITC and objectives of 20× or 60× magnification on a Nikon TE300 microscope with a Leica DC50 color digital camera (Leica, Bannockburn, Ill.) or a CoolSnap HQ Monochrome CCD camera (Photonetrics, Tucson, Ariz.). The Leica DC50 and CoolSnapHQ cameras were controlled with Leica DC50 software and MetaVue/MetaMorph software, respectively (Universal Imaging Corp, Downingtown, Pa.).

Pericentrin and α-Tubulin Immunofluorescence

CV-1 cells, a monkey kidney fibroblast cell line obtained from ATCC, were grown in chamber slides (Labtek, Campbell, Calif.) in culture media containing 90% Eagle's MEM and 10% BCS. Control cells were incubated with DMSO and drug-treated cells were incubated for 5 hours with 0.5 μM Compound 1 or 0.5 μM Taxol. Cells were fixed in 4% paraformaldehyde (Sigma, St. Louis, Mo.) at room temperature for 20 minutes and washed in phosphate buffered saline (PBS). Permeablilization was then performed with 5% Triton X-100 (Sigma, St. Louis, Mo.) in PBS for 10 minutes at room temperature. After fixation, cells were washed twice again in PBS for 5 minutes and blocked with 20% human AB serum (Nabi Diagnostics, Boca Raton, Fla.) in PBS at 37° C. for 20 minutes. Slides were then incubated at 37° C. for 30 minutes with primary antibodies against pericentrin (rabbit polyclonal, 1:500 dilution; Abcam, Cambridge, Mass.) and α-tubulin (mouse monoclonal antibody at 1:1000 dilution, clone DM1A, Sigma, St. Louis, Mo.). Subsequently, slides were washed in PBS and incubated with a Cy3-conjugated goat anti-rabbit secondary antibody (1:500 dilution, Jackson Immunoresearch Laboratories, West Grove, Pa.) or AlexaFluoR₄₈₈ goat anti-mouse secondary antibody (1:1000 dilution; Molecular Probes, Eugene, Oreg.) at 37° C. for 30 min. Slides were counterstained with 0.5 g/ml of 4′,6-diamidino-2-phenylindole (DAPI, Molecular Probes, Eugene, Oreg.) in PBS at room temperature for 10 minutes, and mounted in ProLong mounting medium (Molecular Probes, Eugene, Oreg.). Preparations were visualized on a Nikon E800 microscope (Nikon, Melville, N.Y.) and images were recorded with a CCD camera (Sensicam; Cooke Corp., Auburn Hills, Mich.).

α-tubulin and γ-tubulin Immunofluorescence

CHO cells expressing alpha-tubulin-YFP were grown in chamber slides (Labtek, Campbell, Calif.) in culture media containing 90% HamF12, 10% FBS and 1% G418. Control cells were incubated with DMSO and drug-treated cells were incubated for 5 hours with 10 nM Taxol+0.5 μM Compound 1. Cells were fixed in 4% paraformaldehyde (Sigma, St. Louis, Mo.) at room temperature for 20 minutes and washed in PBS. Permeablilization was then performed with 5% Triton X-100 (Sigma, St. Louis, Mo.) in PBS for 10 minutes at room temperature. After fixation, cells were washed twice again in PBS for 5 minutes and blocked with 20% human AB serum (Nabi Diagnostics, Boca Raton, Fla.) in PBS at 37° C. for 20 min. Slides were then incubated at 37° C. for 30 minutes with a monoclonal mouse anti-gamma-tubulin antibody at a 1:1000 dilution (clone GTU-88, Sigma, St. Louis, Mo.). Subsequently, slides were washed in PBS and incubated with a goat anti-mouse Cy3-conjugated secondary antibody at a 1:500 dilution (Jackson Immunoresearch Laboratories, West Grove, Pa.) at 37° C. for 30 minutes. Slides were counterstained with 0.5 g/ml of 4′,6-diamidino-2-phenylindole (DAPI, Molecular Probes, Eugene, Oreg.) in PBS at room temperature for 10 minutes, and mounted in ProLong mounting medium (Molecular Probes, Eugene, Oreg.). Preparations were visualized on a Nikon E800 microscope (Nikon, Melville, N.Y.) and images were recorded with a CCD camera (Sensicam, Cooke Corp., Auburn Hills, Mich.).

Results

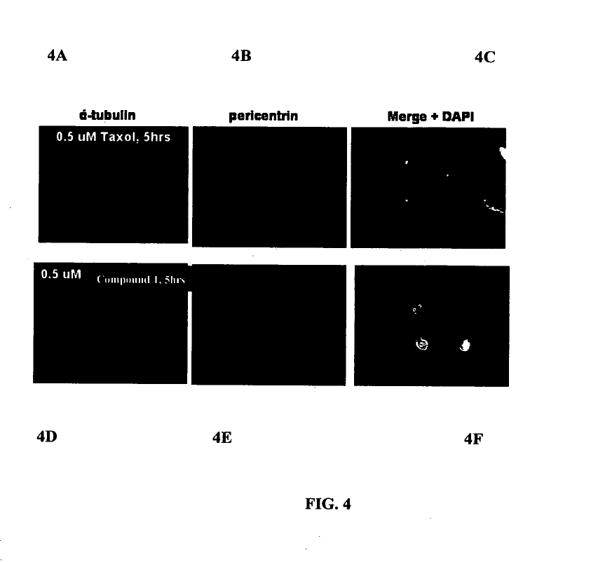

Compound 1 treatment of cultured cells had a profound effect on centrosome structure (see, e.g., FIGS. 1-4). Experiments using α-tubulin-YFP-transfected CHO cells demonstrated that Compound 1 treatment caused a time-dependent accumulation of α-tubulin-YFP at the centrosomes (FIGS. 1 and 2). The first sign of tubulin accumulation was seen 5 hours after treatment (FIG. 1C). When used in combination with 10 nM Taxol, the accumulation of tubulin at the centrosome appeared earlier (at 2 hours) and was even more prominent at 5 hours (FIG. 1D). Longer treatment with Compound 1 alone or in combination with Taxol resulted in a more frequent and greater accumulation of tubulin at the centrosome (FIGS. 2C and 2D). Cells treated with Compound 1 alone or in combination with Taxol also showed a more diffuse and intense pattern of staining for pericentrin, a highly conserved centrosomal protein (FIG. 4E).

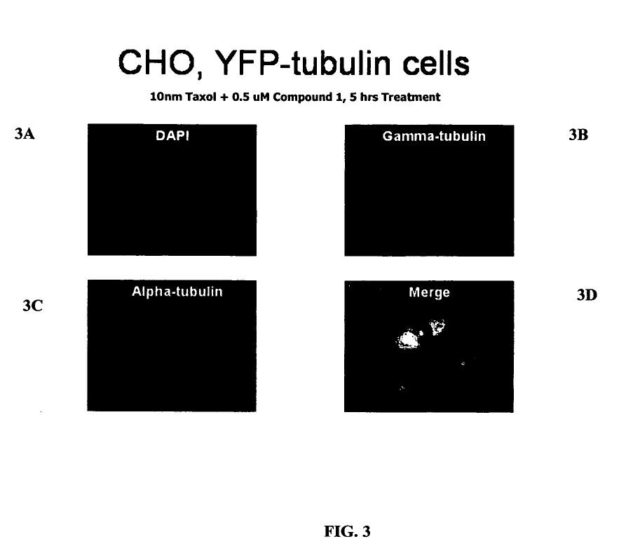

The same morphological changes were observed when direct staining of endogenous α-tubulin in CHO cells was performed. As depicted in FIG. 3D, gamma-tubulin, a centrosomal marker, co-localized with the tubulin-YFP, thereby confirming that the labeled tubulin was accumulating at the centrosomes. Only Compound 1 and Compound 1 plus Taxol caused an accumulation of tubulin in the centrosomes (FIGS. 1 and 2), while DMSO and Taxol alone had no such effect.

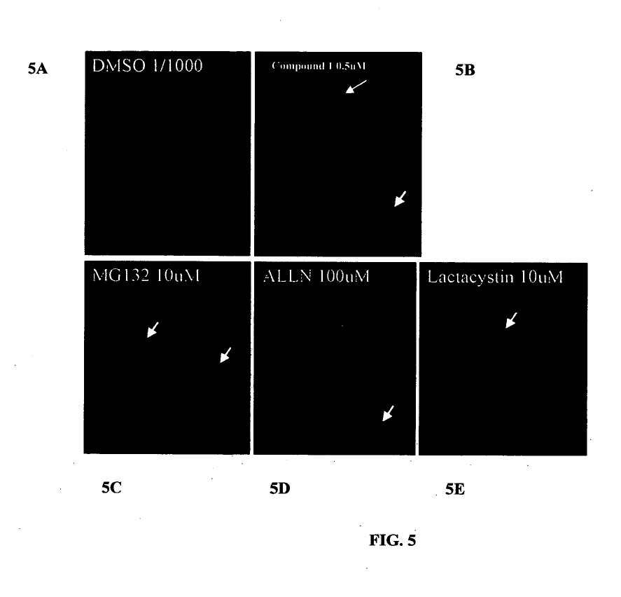

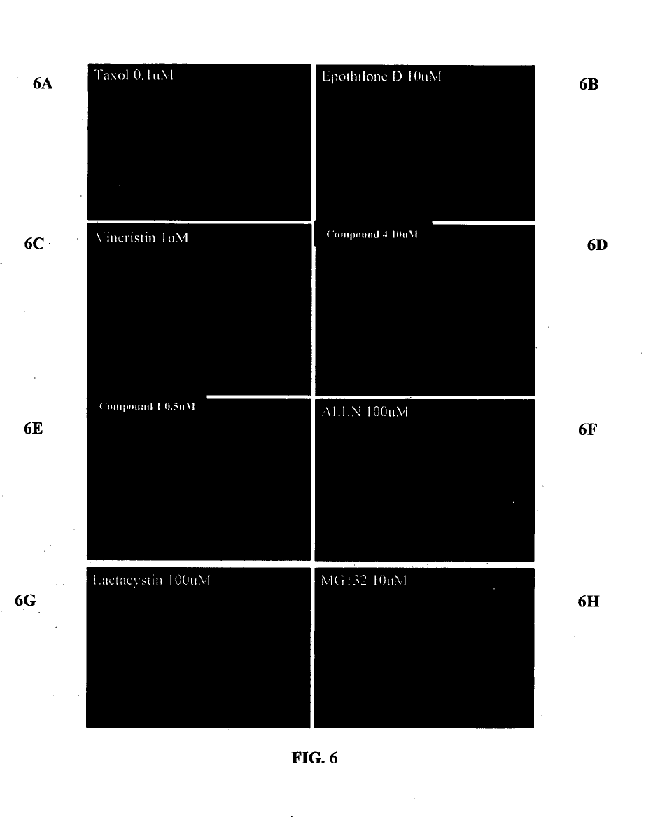

Furthermore, the accumulation of tubulin in centrosomes correlates with an inhibition of protein degradation. The proteasome inhibitors, ALLN, Lactacystin and MG132, were individually used to inhibit protein degradation in CHO-tubulin-YFP cells. Like Compound 1 (FIGS. 5B, 6E, 7A and 7B), each of the inhibitors caused accumulation of tubulin-YFP at the centrosomes and in the perinuclear region (FIGS. 5C-5E, 6B, 6C, 6F-6H and 7C). Accumulation of tubulin-YFP was also found in the nucleoli in cells treated with known proteasome inhibitors, a phenotype that was not observed following Compound 1 treatment (see Example 5).

### Example 2

### Compound 1 Does not Inhibit the Activity of Isolated Proteasomes in Vitro

Materials and Methods

20S Proteasome Assay

190 μl of reaction buffer (500 mM HEPES, 10 nM EDTA, pH 7.6), containing 0.03% SDS was pre-incubated for 5 minutes at 37° C. in the presence of 0.2 μg of bovine red blood cell 20S proteasome (Calbiochem, San Diego, Calif.) for temperature equilibration. Subsequently, inhibitors or Compound 1 were added to the reaction mixture at a final DMSO concentration of 0.5%. The reaction was initiated by adding 10 μl of the peptide-AMC substrate (Calbiochem, San Diego, Calif.) to each well. The emitted fluorescence was then measured every third minute at 37° C. for 90 minutes by a fluorescence plate reader (FlexStation II, Molecular Devices, Sunnyvale, Calif.) at 460 nm (λₑₓ360 nm) wavelength. The effects of both high (50 μM) and low (5 nM) concentrations of Compound 1 on proteasome activity were examined.

Results

As proteasome inhibitors and Compound 1 both induce accumulation of tubulin in the centrosomal region, the ability of Compound 1 to inhibit proteasome activity was tested using an in vitro assay that monitors degradation of the fluorogenic substrate Suc-Leu-Val-Tyr-AMC by an SDS-activated proteasome. The buffers and reagents for the assay were purchased from Calbiochem (20S Proteasome Assay Kit; Calbiochem, San Diego, Calif.). The principle of the assay is that the release of free AMC (7-Amino-4-methylcoumarin) following degradation of the substrate results in a fluorescent signal that is a measure of proteasome activity. The quantitative analysis of 20S proteasome activity was assayed as described.

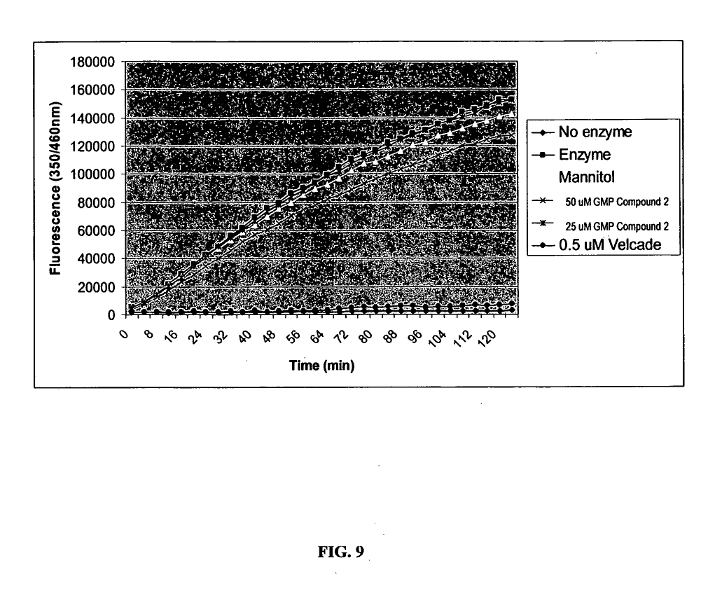

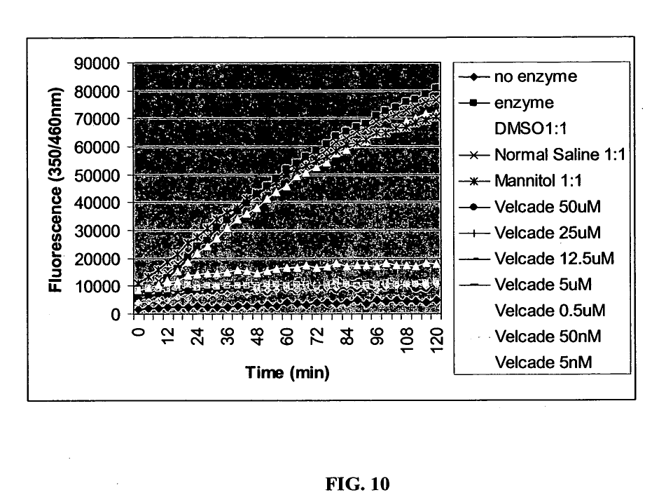

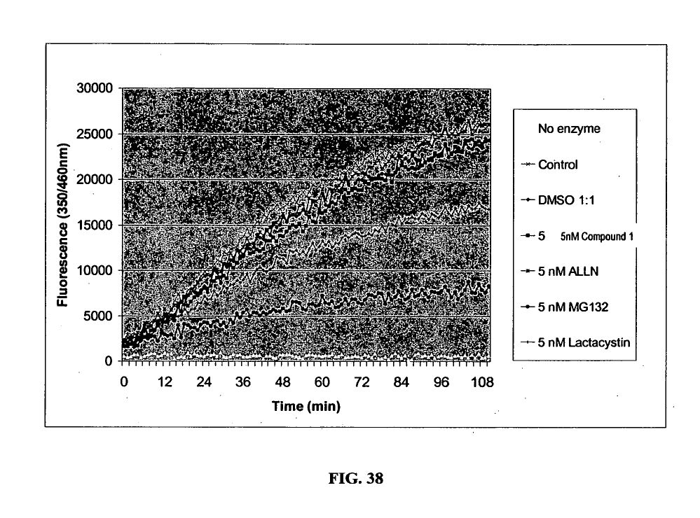

This in vitro assay indicated that, at lower concentrations (5 nM), the proteasome inhibitors, MG132 and lactacystin, each induced a detectable decrease in proteasome activity, while Compound 1 had no effect on proteasome activity (FIG. 38). Even at a high concentration (50 μM), neither Compound 1 nor its salt form, Compound 2, displayed significant proteasome inhibitory activity (FIGS. 8 and 9), while 0.5 μM Velcade (PS-341; Millennium Pharmaceuticals Inc., Cambridge, Mass.), which was used as a positive control, almost completely inhibited the proteasome (FIG. 10).

### Example 3

### Compound 1 Inhibits Proteasome Activity in Cell-Based Assays

Materials and Methods

To test proteasome inhibitory activity of Compound 1 in living cells, a HEK-293 cell line that expresses a proteasome-targeting GFP chimera protein was utilized (the proteasome-sensor cells). Specifically, the proteasome-sensor cells are HEK-293 cells stably transfected with a vector (proteasome-sensor vector) that encodes naturally-occurring reef coral Zoanthus sp. green fluorescent protein (GFP) fused to a specific degradation motif that targets the fusion protein for removal by the 26S proteasome. The background fluorescence observed in normal cells with active proteasomes is low. When proteasomes are inhibited, the fluorescent protein quickly accumulates. Proteasome-sensor cells were treated with various concentrations of Compound 1 and Drug-V (Velcade; Millennium Pharmaceuticals, Inc., Cambridge, Mass.).

To determine if the proteasome inhibitory effect of Compound 1 is dose dependent, proteasome-sensor cells were cultured in 2-well-chambered coverslips for 24-48 hours until they reached 70% confluence, and then treated with DMSO alone, or Compound 1 (5 nM, 50 nM, 500 nM or 5 μM) for 20 hours. Velcade (Millennium Pharmaceuticals Inc.) was used as a positive control in this assay (at 5 nM, 50 nM, 500 nM or 5 μM). Velcade stock solution was prepared according to the manufacturer's instructions. GFP fluorescence of the cells was imaged at various time points using a standard filter for FITC with the Nikon TE300 microscope/digital imaging system described herein.

To measure the proteasome-inhibition effect of Compound 1, GFP fluorescence in the proteasome-sensor cells treated with Compound 1 and Velcade was measured using flow cytometry. Proteasome-sensor cells were cultured in 100 mm dishes for 24-48 hours until they reached 70% confluence and then were treated with DMSO alone, 500 nM of Compound 1 or 100 nM of Velcade for 24 hours. Cells were harvested by treating the cells with 1×PBS for 5 minutes and pipetting the cells up and done 10 times. The cells were passed through a 100 μm-diameter filter before analyzing by FACS. A standard FITC filter was used for the FACS analysis. Quantitation of the proteasome-inhibitory effect of Compound 1 was determined by an increase in the percentage of GFP-positive cells, as compared to treatment with DMSO alone.

To further characterize the proteasome-inhibitory effect of Compound 1 in live cells, proteasome-sensor cells were treated with Compound 1 and the accumulation of the GFP-based proteasome substrate was measured by flow cytometry.

Results

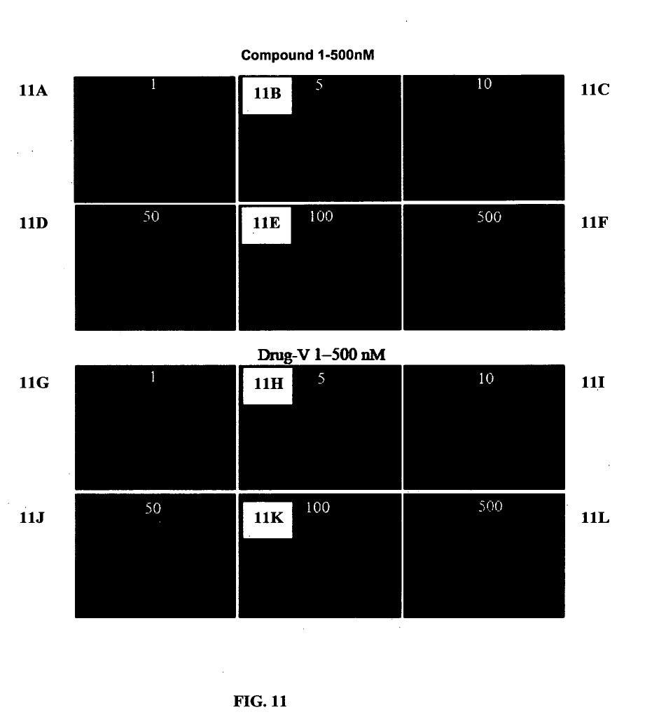

Treatment of the proteasome-sensor cells with 50 nM of Compound 1 induced an increase of the GFP-based proteasome substrate in the cytoplasm in some scattered cells (FIG. 11D). Treatment with 100 nM of Compound 1 significantly increased the GFP signal in the cytosol (FIG. 11E), and treatment with 500 nM of Compound 1 resulted in even greater GFP signal (FIG. 11F). Velcade (Drug-V) at 5 nM significantly induced an increase of GFP signal (FIG. 11H). This data confirms that Compound 1 has moderate proteasome inhibition activity in this cell-based assay. One possibility is that inhibition of proteasome activity of Compound 1 is directly linked to its mechanism of action in vivo.

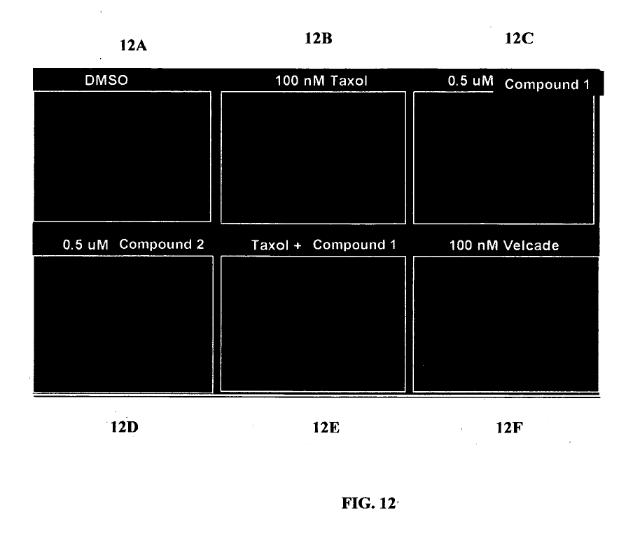

For the flow cytometry assay, the following non-gated data demonstrates that treatment with either Compound 1 (500 nM) or Velcade (100 nM) caused a significant increase of fluorescence 20 hours after treatment (FIGS. 13B and 13C; populations designated by red arrows, or the LR values). Although the final concentration of Compound 1 is 4-fold higher than that of Velcade, the increase in fluorescent cell population is about 4-fold lower than that of cells treated with Velcade (FIGS. 13B and 13C). This is consistent with the previous microscopy results showing that Compound 1 is a weak/moderate proteasome inhibitor (FIGS. 11 and 12).

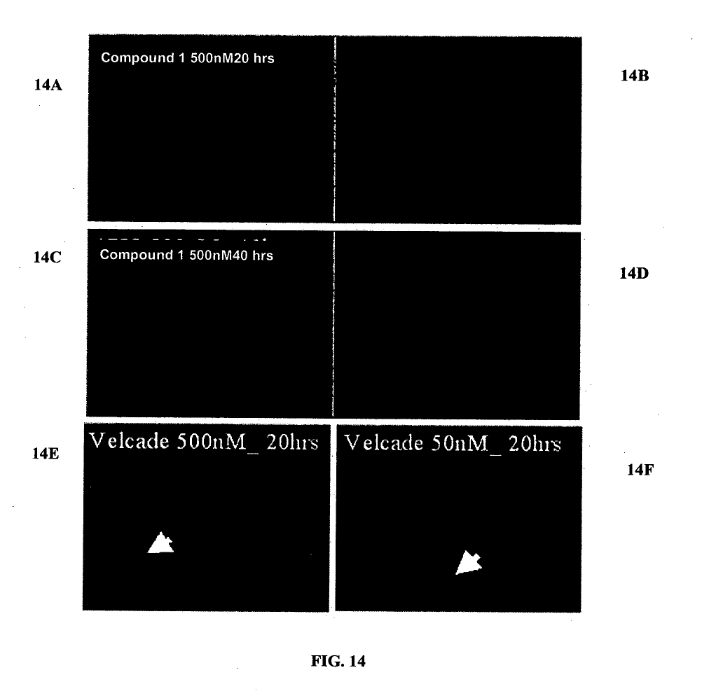

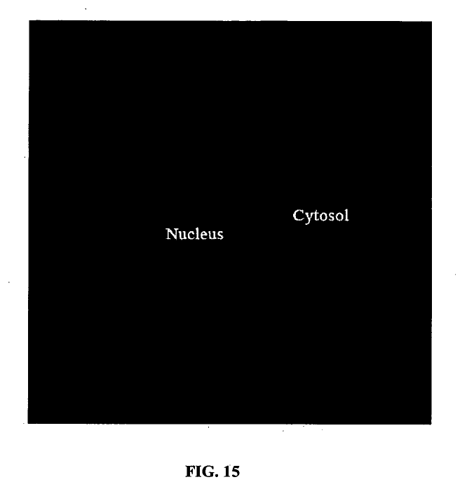

In addition, Compound 1 shows a greater proteasome inhibitory effect in cells at the periphery of a colony than in cells in the center of a colony (FIGS. 14A-14D). It was previously noted that Compound 1 had less effect on cells that are growing in the center of cultured colonies. Using the proteasome-sensor cell line described above, the accumulation of the GFP-based proteasome substrate was measured in cells at the periphery and in cells at the center of cultured colonies. At the periphery of the colonies, Compound 1 caused significant accumulation of the GFP-based proteasome substrate (FIG. 14A-14D; red arrows), while at the center of the colonies, Compound 1 did not cause significant accumulation (FIG. 14A-14D; blue arrows). As a comparison, treatment with Velcade resulted in greater accumulation of the GFP-based proteasome substrate in cells at the colony center than did Compound 1 (FIGS. 14E and 14F; yellow arrows). The decreased proteasome inhibitory activity of Compound 1 in cells at the colony center correlated with a decreased cell-killing effect on those cells. High-resolution images of the proteasome-sensor cells indicate that the proteasome-sensor protein was distributed generally (FIG. 15). The broad distribution of the proteasome substrate suggests that the downstream effect of proteasome inhibition by Compound 1 may affect other intracellular organelles.

GMP-grade Compound 1 failed to inhibit isolated proteasomes when tested using the above-described in vitro proteasome assay (FIGS. 8 and 9). However, GMP-grade Compound 1 did exhibit a detectable level of proteasome inhibition activity when tested using cell-based assays, as described above. The discrepancy between the results from the in vitro and in vivo proteasome assays could indicate that Compound 1 is being activated within cells and/or exerts its effect through proteasome regulators that are absent in the in vitro proteasome assay. This discrepancy further indicates that Compound 1 may represent a novel class of proteasome inhibitor that has not been reported to our knowledge. This theory is further strongly supported by indirect evidence of Compound 1 affecting microtubule and actin cytoskeletons that are not affected by known proteasome inhibitors we tested.

### Example 4

### Compound 1 Disrupts Organization of the Cytoskeleton Microtubule and Actin Networks and Affects Cell Morphology

i) Microtubule Network

Materials and Methods

α-tubulin Immunofluorescence

CV-1 cells (a monkey kidney fibroblast cell line) were selected to study the effects of Compound 1 on microtubule and centrosome structure because of their large size and flat morphology. Before treatment, cells were grown in chamber slides for 24 hours in culture media containing 90% Eagle's MEM and 10% BCS. Control cells were incubated with DMSO and drug-treated cells were incubated for 5 hours in the presence of 0.5 μM of Compound 1. Cells were then washed in PBS and fixed in ice-cold 50/50 methanol/acetone for 10 minutes. After fixation, cells were washed twice again in PBS for 5 minutes and blocked with 20% human AB serum (Nabi Diagnostics, Boca Raton, Fla.) in PBS at 37° C. for 20 minutes. Slides were then incubated at 37° C. for 1 hour with a monoclonal anti α-tubulin antibody at a 1:500 dilution (clone DM1A; Sigma, St. Louis, Mo.). Subsequently, slides were washed in PBS, incubated with an AlexaFluoR488-conjugated anti-mouse antibody (Molecular Probes, Eugene, Oreg.) at 37° C. for 30 minutes. Slides were counterstained with 0.5 g/ml 4′,6-diamidino-2-phenylindole (DAPI, Molecular Probes, Eugene, Oreg.) in PBS at room temperature for 10 minutes, and mounted in ProLong mounting medium (Molecular Probes, Eugene, Oreg.). Preparations were visualized on a Nikon E800 microscope (Nikon, Melville, N.Y.) and images were recorded with a CCD camera (Sensicam, Cooke Corp., Auburn Hills, Mich.).

Results

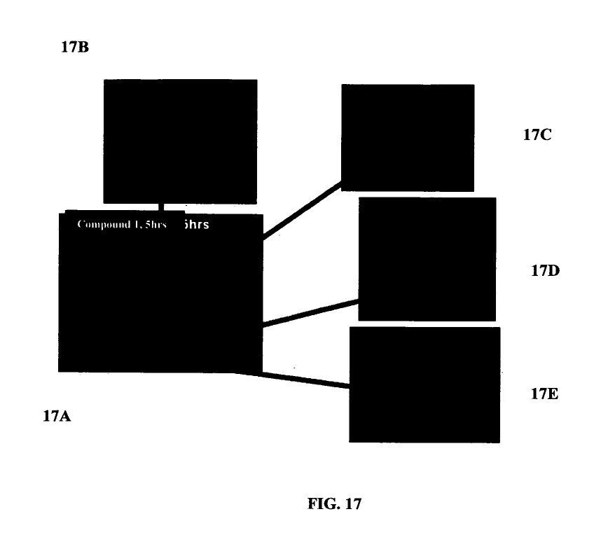

Compound 1 had a dramatic effect on the organization of the microtubule network resulting in loss of centrosomal enucleated microtubules, clearing up of microtubules from the cytosol, and coiling up of microtubules around the nucleus and at the cell periphery (FIGS. 18C, 18D and 21B). In DMSO-treated cells, cytoplasmic microtubules radiate from the centrosomal region and extend to the periphery of the cytoplasm (FIG. 16A-16C). However, Compound 1-treated cells (0.5 μM, 5 hours) display a remarkably different pattern of microtubule network that is unevenly distributed throughout the cytoplasm (FIG. 16D-16F). This disrupted network of microtubules is sparse around the periphery of the cytoplasm but dense and clustered around the perinuclear region (FIGS. 16D-16F and 17A-17F).

ii) Actin Network

Materials and Methods

Actin Immunofluorescence

CV-1 cells were obtained from ATCC and grown in chamber slides (Labtek, Campbell, Calif.) for 24 hours in 90% Eagle's MEM with 10% BCS before treatment. Control cells were incubated with DMSO, while drug-treated cells were incubated in the presence of 0.5 μM Compound 1, 100 nM Taxol or the combination of these two drugs (0.5 μM Compound 1+100 nM Taxol) for 6 hours. Cells were then washed in PBS and fixed in 3.7% formaldehyde solution in PBS for 10 minutes at room temperature. Cells were washed again twice in PBS for 5 minutes before incubation with AlexaFluoR488 conjugated phalloidin (1:40 dilution; Molecular Probes, Eugene, Oreg.) and 1% bovine serum albumin (BSA) in PBS at 37° C. for 30 minutes. After subsequent washing in PBS, slides were counterstained with 0.5 g/ml 4′,6-diamidino-2-phenylindole (DAPI, Molecular Probes, Eugene, Oreg.) in PBS at room temperature for 10 minutes, and mounted in ProLong mounting medium (Molecular Probes, Eugene, Oreg.). Preparations were visualized on a Nikon E800 (Nikon, Melville, N.Y.) and images were recorded with a CCD camera (Sensicam, Cooke Corp., Auburn Hills, Mich.).

Results

The organization of the actin network, another major cytoskeleton component that is responsible for supporting the cell, determining the shape of the cell and directing movement and division of the cell, was analyzed in CV-1 cells treated with Compound 1. Compound 1 treatment induced the disappearance of cytosolic actin bundles (parallel actin fibers) thereby resulting in a more prominent cortical actin network (the area just below the membrane that contains the actin cytoskeleton) (FIG. 22C). Consistent with previous findings regarding the microtubule network, it was determined that the microtubules are oriented where actin bundles remain, but are absent from areas where actin bundles have disappeared (FIGS. 17 and 22). Simultaneous treatment with Taxol and Compound 1 neither prevented nor modified actin redistribution (FIG. 22D).

iii) Cell Morphology

Materials and Methods

CV-1 cells and CHO cells were previously cultured in 2-well chambered cover-slips for 48 hours and incubated in 15 mM HEPES buffer-containing Ham's F-12/DMEM medium with different compounds. The cells were imaged using phase contrast settings with the Nikon TE300 microscope/digital imaging system. The cells were imaged ‘simultaneously’ under control of MetaVue software by capturing frames for cells from different conditions at the same time period using a motorized automated XYZ stage.

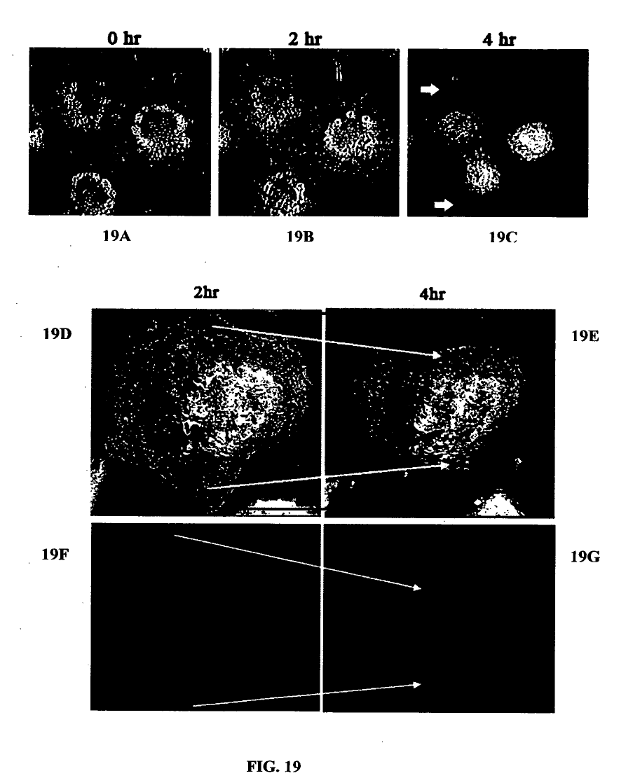

To track the effect of Compound 1 on live cells, non-transfected CV-1 cells with were treated with 500 nM Compound 1 and phase contrast time-lapse images were taken every 10 seconds. Compound 1 caused cell shrinkage beginning 1.5 hours after treatment (FIGS. 19A-19G). Similar to transfected cells, the shrinkage of the cell body (FIG. 19A, red arrows) appears to occur much earlier than the loss of the focal adhesions (FIG. 19A, yellow arrows). Furthermore, Tubulin-YFP-labeled microtubules collapsed between 2 hr and 4 hr following Compound 1 treatment (FIG. 19B). Thus, it is possible that Compound 1 disrupts the cytoskeleton, thereby forcing cells to shrink prior to altering cell adhesion. Compound 1, Velcade, Compound 1+Taxol, and Taxol cause cell death after a certain period of treatment. To determine the relationship between cell shrinkage and cell death and the difference in cellular morphological changes observed among these types of treatment, simultaneous time-lapse imaging techniques were employed to monitor morphological changes in CHO cells treated for up to 8 hours with Compound 1, Velcade, Compound 1+Taxol or Taxol. A higher concentration of Compound 1 (500 nM) than Velcade (100 nM) was used to minimize the difference in proteasome inhibitory activity between these two drugs. A very low concentration of Taxol (10 nM) was used.

Results

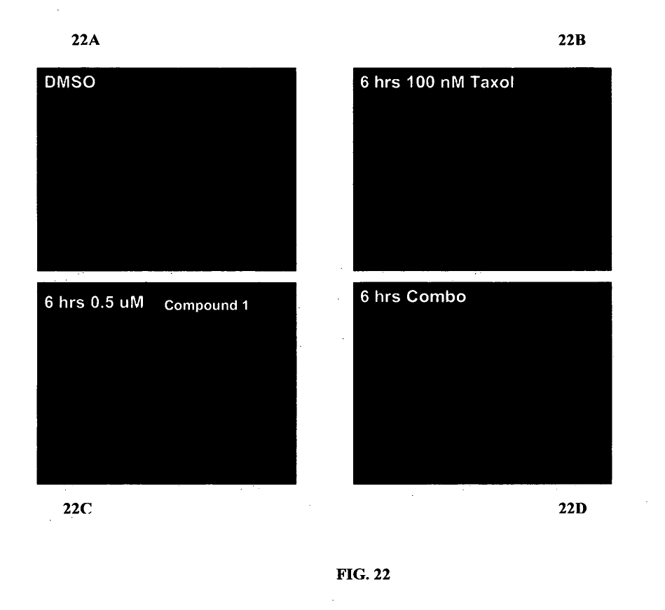

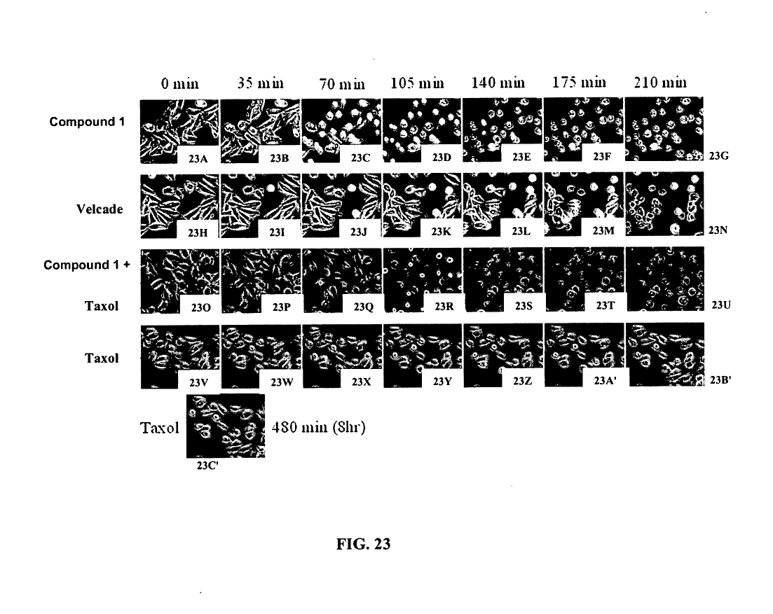

Compound 1 induced cell shrinkage at 70 minutes, while Velcade did not dramatically affect cell morphology until cell death at 210 minutes (FIGS. 23A and 23N). Compound 1 may cause earlier cell shrinkage than Velcade at a comparable proteasome inhibitory activity level. This is consistent with previous results indicating that Compound 1 induces unique cytoskeleton changes, which might contribute to the very early change of cell shape. Compound 1+Taxol induced similar cell shape changes to Compound 1 alone and seemed to cause earlier cell death than Compound 1 alone (FIG. 23O-23U). As a control, Taxol alone did not cause significant cell morphological changes at the concentration and time period tested (FIG. 23V-23B′). Thus, Compound 1 may induce a significantly stronger effect on cell morphology than does Velcade, notwithstanding the fact that Velcade has greater proteasome inhibitory activity.

### Example 5

### Compound 1, Unlike Known Proteasome Inhibitors, Does not Induce Accumulation of Tubulin in the Nucleus

Materials and Methods

CHO-α-tubulin-YFP cells were cultured in 2-well chambered cover-slips (Labtek (Campbell, Calif.) or Fisher Scientific) 24 hours before treatment. For comparison of effects on the centrosome, nucleus, and perinuclear regions, cells were treated with Compound 1 and known proteasome inhibitors including ALLN, MG132, Lactacystin, MG115, clasto-Lactacystin β-Lactone (cLβL), and Epoxomicin (10 μM final concentration for all except ALLN, which was used at 100 μM final concentration) (proteasome inhibitors were from Calbiochem, San Diego, Calif.) and imaged at various time points up to 24 hours during treatment.

Tubulin-YFP fluorescence in living cells or fixed cells was captured using a standard filter for FITC and objectives of 20× or 60× magnification on the Nikon TE300 microscope/digital imaging system.

Results

In this study, the accumulation of YFP-tubulin in the nucleus of cells that were treated with various proteasome inhibitors was observed (FIGS. 24 and 25). At 8 hours post-treatment, all of the proteasome inhibitors that were tested, including ALLN, MG132, Lactacystin, MG115, clasto-Lactacystin β-Lactone (cLβL), and Epoxomicin, caused the accumulation of YFP-tubulin in the nucleus (FIG. 24A-24F, 25D, 26D and 26E). In contrast, Compound 1 treatment did not result in any nuclear accumulation of YFP-tubulin (FIGS. 24K, 25A, 26A and 26B). Thus, the effects of Compound 1 and known proteasome inhibitors on the redistribution of YFP-tubulin in cells (e.g., CHO cells) are not identical.

### Example 6

### Compound 1 is a Potent Hsp70 Inducer Relative to Known Proteasome Inhibitors

Materials and Methods

Hsp70 Western Blotting

MDA-435 breast cancer cells were grown in 100 mm plastic-tissue culture dishes and treated for 6 and 24 hours with 0.5 μM Taxol, Compound 1, Taxol+Compound 1, MG132, ALLN, or DMSO. After treatment, cells were washed in PBS and cell lysates were prepared by adding 100 μL of lysis buffer, which contained 20 mM Tris-HCl (pH 7.5), 150 mM NaCl, 1 mM Na₂EDTA, 1 mM EGTA, 1% Triton, 2.5 mM sodium pyrophosphate, 1 mM β-glycerophosphate, 1 mM Na₃VO₄ and 1 μg/ml leupeptin (Cell Signaling Technology, Beverly, Mass.) supplemented with 1 mM PMSF (Sigma, St. Louis, Mo.) immediately before use, on ice for 1 hour. After scraping, the lysates were cleared by centrifugation at 13,000 RPM for 10 minutes at 4° C. Protein content was determined using a Bradford assay and bovine serum albumin as a standard (Bio-Rad Laboratories, Hercules, Calif.). Samples were solubilized by boiling in SDS sample buffer and subjected to SDS-PAGE. The proteins were subsequently transferred to a PVDF membrane (Bio-Rad, Hercules, Calif.). After blocking with 5% nonfat milk in TBS for 1 hour, the blots were incubated with a mouse monoclonal anti-Hsp70 antibody, which is specific for the inducible form of Hsp70 (Clone C92F3A-5, 1:1000 dilution, Stressgen Biotechnologies Corp., Victoria, B.C., Canada) or a rabbit polyclonal anti-GAPDH antibody (1:2000 dilution, Abcam, Cambridge, Mass.) for 2 hours at room temperature. The horseradish-peroxidase-conjugated secondary antibodies, anti-mouse horse radish peroxidase, (1:2000, Bio-Rad, Hercules, Calif.) and goat anti-rabbit polyclonal horse radish peroxidase (1:2000, Abcam, Cambridge, Mass.)) were diluted in blocking buffer and incubated with the blot for 1 hour at room temperature. The secondary antibodies were detected by enhanced chemiluminescence (ECL, Amersham Biosciences, Piscataway, N.J.) and imaged using a Kodak 440 Image Station.

Results

Compound 1 alone, and in combination with Taxol, strongly induced Hsp70 expression after 6 and 24 hours of treatment (FIG. 27). MG132 also induced Hsp70 expression after 6 and 24 hours of treatment, but did so less potently than Compound 1 or Compound 1+Taxol (FIG. 27). ALLN was not capable of inducing Hsp70 expression at the tested dose (FIG. 27).

### Example 7

### Compound 1 Induces Accumulation of Multi-Ubiquitinated Proteins in Living Cells

Materials and Methods

Multi-Ubiquitin Western Blotting

MDA-435 breast cancer cells were grown in 100 mm plastic-tissue culture dishes and treated for 6 and 24 hours with 0.5 μM Taxol, Compound 1, Taxol+Compound 1, MG132, ALLN, Lactacystin or DMSO. After treatment, cells were washed in PBS and cell lysates were prepared by adding 100 μL of lysis buffer, which contained 20 mM Tris-HCl (pH 7.5), 150 mM NaCl, 1 mM Na₂EDTA, 1 mM EGTA, 1% Triton, 2.5 mM sodium pyrophosphate, 1 mM β-glycerophosphate, 1 mM Na₃VO₄ and 1 μg/ml leupeptin (Cell Signaling Technology, Beverly, Mass.) supplemented with 1 mM PMSF (Sigma, St. Louis, Mo.) immediately before use, on ice for 1 hour. After scraping, the lysates were cleared by centrifugation at 13,000 RPM for 10 minutes at 4° C. Protein content was determined using a Bradford assay and bovine serum albumin as a standard (Bio-Rad Laboratories, Hercules, Calif.). Samples were solubilized by boiling in SDS sample buffer and subjected to SDS-PAGE. The proteins were subsequently transferred to a PVDF membrane (Bio-Rad, Hercules, Calif.). After blocking with 5% nonfat milk in TBS for 1 hour, the blots were incubated with a mouse monoclonal anti-multi ubiquitin antibody (Clone FK2, 1:1000 dilution, MBL International, Woburn, Mass.), specific for multi-ubiquitin chains or a rabbit polyclonal anti-GAPDH antibody (1:2000, Abcam, Cambridge, Mass.), for 2 hours at room temperature. The alkaline phosphatase-conjugated secondary antibodies (Invitrogen, Carlsbad, Calif.) were diluted in blocking buffer and incubated with the blot for 1 hour at room temperature. The secondary antibodies were detected by the Western Breeze Chemiluminescent Kit (Invitrogen, Carlsbad, Calif.) and imaged using a Kodak 440 Image Station.

Results

Proteins targeted for degradation by the ubiquitin-dependent proteolytic pathway are tagged with multi-ubiquitin molecules. Western blot analysis using an antibody that specifically recognizes multi-ubiquitin chains was performed to test the effect of Compound 1 in a non-GFP-dependent cell-based assay system. In this assay, accumulation of multi-ubiquitinated proteins in the cells is indicative of the impairment of proteasome activity.

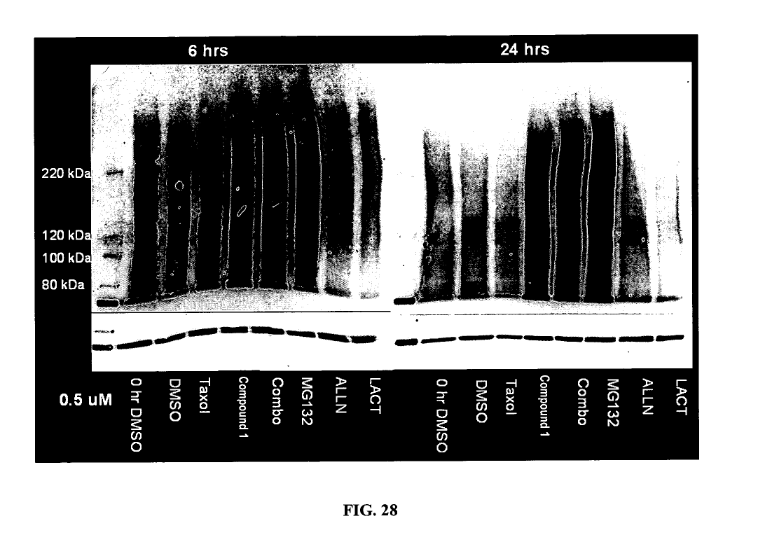

Compound 1, alone and in combination with Taxol, induced the accumulation of multi-ubiquitinated proteins in MDA-435 cells, thereby indicating a detectable level of proteasome inhibition (FIGS. 28 and 29). The level of proteasome inhibition achieved with 0.5 μM Compound 1 was below that of the positive control (500 nM and 5 nM Velcade) (FIG. 29). In addition, among the tested proteasome inhibitors, only MG132 caused similar accumulation of multi-ubiquitinated proteins (FIG. 28). The lack of activity of several known proteasome inhibitors in the cell-based assay suggests that they are not readily available for the cells and/or that they become rapidly inactivated. Although Compound 1 showed a very weak proteasome inhibitory activity in the in vitro assay, these results suggest that its cell-based activity is comparable to known proteasome inhibitors.

### Example 8

### Compound 1 Does not Inhibit Aurora-A kinase

Materials and Methods

Aurora-A Kinase Assay

Aurora-A kinase is associated with centrosomes and plays an important role in centrosome function. An in vitro ELISA assay was performed to examine the ability of Compound 1 to inhibit the phosphorylation of the Lats2 substrate by Aurora-A kinase using the Cyclex Aurora-A Kinase Assay/Inhibitor Screening kit (MBL International, Woburn, Mass.). In the assay, the amount of phosphorylated substrate is measured by binding to ST-3B11, an anti-phospho-Lats2 serine83 monoclonal antibody, subsequently incubating with horseradish peroxidase-conjugated anti-mouse IgG, which then catalyzes the conversion of the chromogenic substrate tetra-methylbenzidine (TMB) from a colorless solution to a blue solution. The color is then quantified by spectrophotometry, which reflects the relative amount of Aurora-A activity in the sample.

To examine whether Compound 1 directly inhibits the activity of Aurora-A kinase, 80 μl of the kinase reaction buffer containing 50 μM ATP, was added to each well and supplemented with 50 μM, 5 μM, 0.5 μM or 50 nM of Compound 1. The assay was then performed according to the manufacturer's instructions using 40 Units/well of recombinant Aurora A enzyme. In the ‘enzyme control’ Aurora A enzyme and the ‘ATP minus control’, ATP was omitted from the reaction mixture. The ‘positive control’ contained all assay components but did not contain drug (Compound 1). The color was quantified using a spectrophotometer (Perkin Elmer HTS 7000 Bio Assay Reader) at 450/535 nm wavelengths.

Results

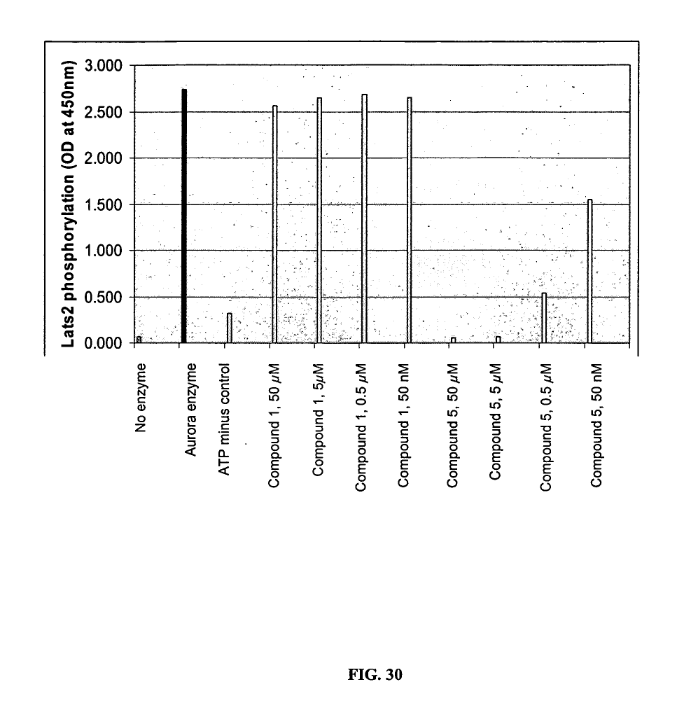

At all concentrations tested (50 nM to 50 μM), Compound 1 did not inhibit the phosphorylation of Lats2 by Aurora-A kinase (FIG. 30). In contrast, the Aurora-A kinase inhibitor, Compound 5 (Aurora kinase inhibitor VX-680; Vertex Pharmaceuticals, Inc., Cambridge, Mass.), inhibited the phosphorylation activity of Aurora-A kinase. Thus, Compound 1 does not appear to inhibit Aurora-A kinase directly.

### Example 9

### Effects of Compound 1 on Tubulin Polymerization

Materials and Methods

Tubulin Polymerization Assay

For in vitro tubulin polymerization assays, lyophilized bovine microtubule-associated protein (MAP)-free tubulin and PEM buffer (80 mM Na-PIPES (pH 6.9), 1 mM MgCl₂, 1 mM EGTA) were purchased from Cytoskeleton (Denver, Colo.). MAP-free tubulin (1.5 mg/ml) was incubated with the test compounds, 0.5 μM Compound 1, 3 μM Taxol, 0.5 μM Compound 1+3 μM Taxol or 0.5 μM Compound 1+30 nM Taxol in PEM-0.3% DMSO. Absorbance at 340 nm was measured every minute for 60 min at 37° C. using a Perkin Elmer HTS 7000 spectrophotometer.

MAP-Rich Tubulin Polymerization Assay

In vitro tubulin polymerization assays were repeated in the presence of microtubule-associated proteins (MAPs). Lyophilized bovine brain microtubule-associated protein (MAP)-rich tubulin and PEM buffer (80 mM Na-PIPES (pH 6.9), 1 mM MgCl₂, 1 mM EGTA) were purchased from Cytoskeleton (Denver, Colo.). MAP-rich tubulin (0.75 mg/ml) was incubated with the test compounds, 0.5 μM Compound 1, 3 μM Taxol, 0.5 μM Compound 1+3 μM Taxol or 0.5 μM Compound 1+30 nM Taxol in PEM-0.3% DMSO. Absorbance at 340 nm was measured every minute for 60 minutes at 37° C. using a Perkin Elmer HTS 7000 spectrophotometer.

Results

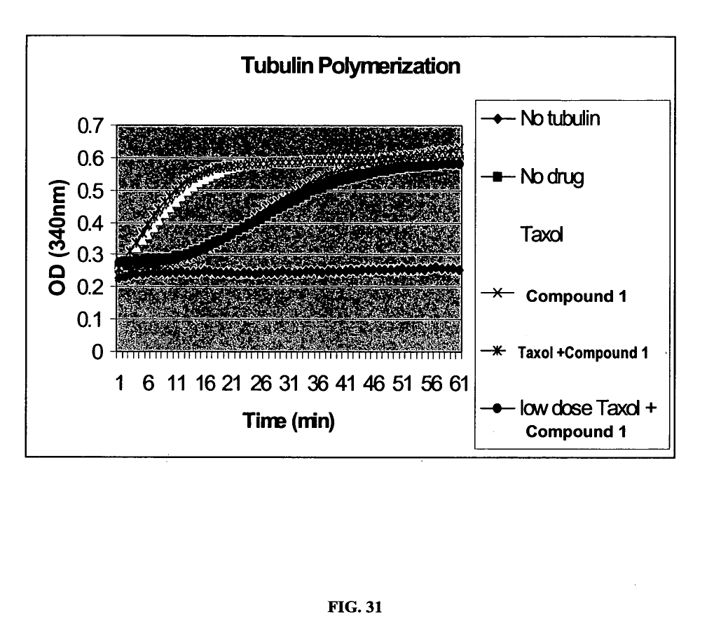

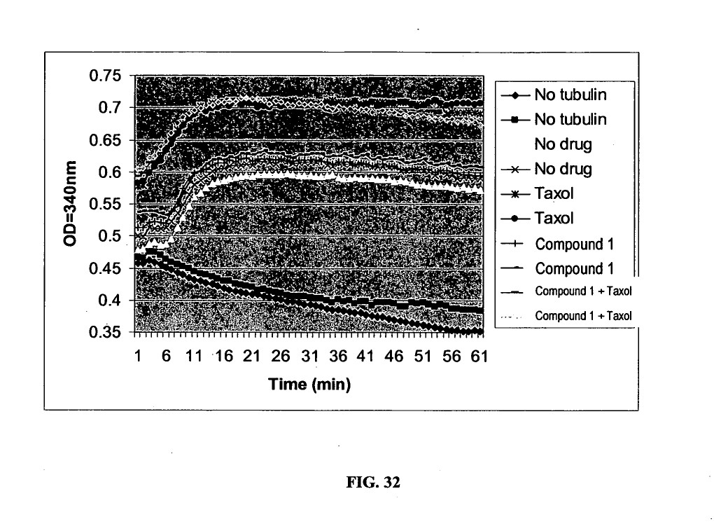

An in vitro tubulin polymerization assay using pure bovine brain tubulin showed that Compound 1 (0.5 μM) had no effect on the kinetics of tubulin polymerization (FIGS. 31 and 32). In addition, Compound 1 did not influence the effect of either high dose (3 μM) or low dose (30 nM) of Taxol on tubulin polymerization (FIGS. 31 and 32). The results suggest that Compound 1 alone or in combination with Taxol does not influence MAP-enriched tubulin polymerization.

### Example 10

### Taxol is Targeted to the Centrosomes in CHO Cells in an Compound 1—Independent Manner

Materials and Methods

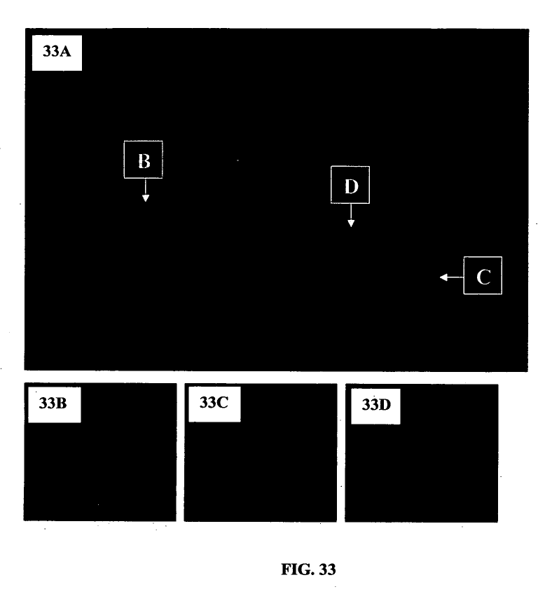

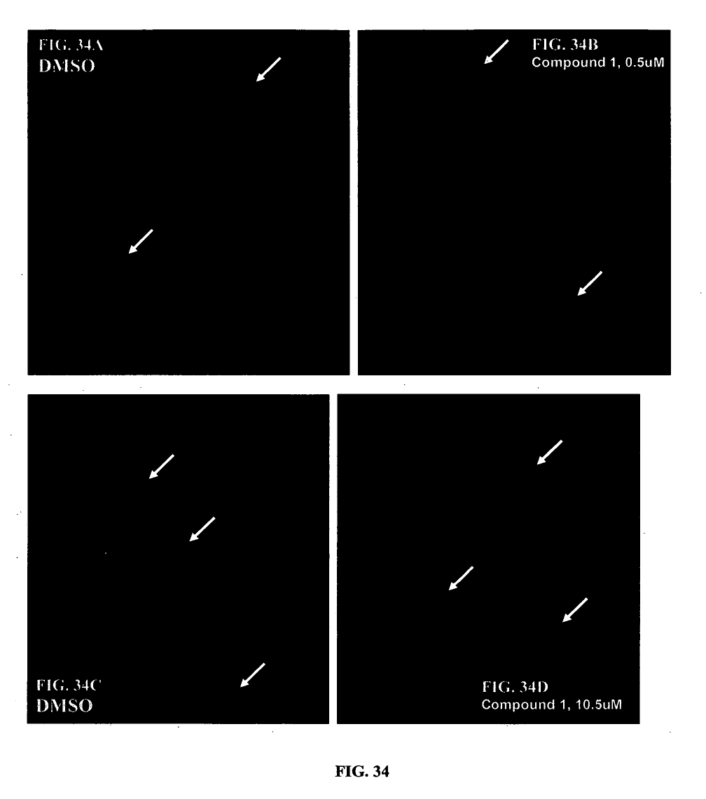

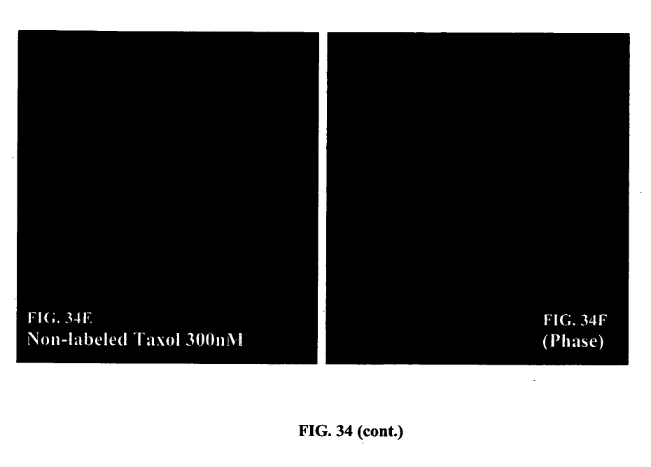

A fluorescent compound, Oregon Green 488-Taxol (Molecular Probes/Invitrogen), was utilized to determine the distribution of Taxol in CHO cells (FIG. 33). In addition, the targeting of fluorescent Taxol to centrosomes was examined in Compound 1-treated cells (FIG. 34). Non-transfected wild-type CHO cells and HeLa cells were cultured in 2-well chambered cover-slips for 24-48 hours before treatment. Oregon Green 488-Taxol stock solution was made with DMSO. The cells were incubated with 1 μM Oregon Green 488-Taxol, 1 μM Taxol alone, or an equivalent concentration of DMSO for 1 hour. In addition, CHO cells were treated with 500 nM of Compound 1 for 3-5 hours prior to Oregon Green 488-Taxol treatment to see if the Compound 1 treatment induced greater accumulation of Oregon Green 488-Taxol. Oregon Green 488-Taxol fluorescence in living cells or fixed cells was imaged using a standard filter for FITC with the Nikon TE300 microscope/digital imaging system.

Results

Experiments utilizing fluorescently-tagged Taxol showed that Taxol localizes to the centrosomal region (FIG. 33). Oregon Green-Taxol localized to microtubules (FIG. 33A), mitotic midbodies (FIG. 33D) and most likely the centrioles found within the centrosomal region (FIGS. 33B and 33C). In both HeLa and CHO cells, Compound 1 had no effect on the accumulation of Taxol at centrosomes (FIG. 34). Given that Compound 1 has a dramatic impact on centrosomal region organization, the centrosomes could be the site of Compound 1/Taxol synergy.

### Example 11

### Generation of Cell Lines for Compound 1 Microtubule Studies

Materials and Methods

MDA-435, MCF-7, CV-1, HT-29 and MCF-10A cell lines were maintained with 10% FBS-containing DMEM media. Cells of low density (˜20%) growing on 2-well chambered cover-slips (Labtek (Campbell, Calif.) or Fisher Scientific) were transfected with a mammalian expression vector encoding α-tubulin-YFP (Clontech, Palo Alto, Calif.) with the use of FuGENE 6 (Roche Molecular Biochemicals, Basel, Switzerland), according to the manufacturer's instructions. 24 hours after transfection, the cells were cultured in 400 μg/ml of G418-(Invitrogen, Carlsbad, Calif.) containing selection medium for 2 weeks. Living cells were examined using a fluorescent microscope for α-tubulin-YFP expression. Cells in single colonies that had microtubules labeled with tubulin-YFP were lifted and expanded using G418-containing media.

Results

MCF-7 and CV-1 cells were transfected successfully with α-tubulin-YFP vector and highly expressing clones were produced. In addition, a CV-1 cell line expressing alpha-tubulin-YFP was successfully generated.

Using a similar strategy, MDA-435, HT-29 and MCF-10A cell lines that express alpha-tubulin-YFP can be produced.

### Example 12

### Compound 1 Selectively Affects Blood Cancer Cell Lines

Materials and Methods

The viability and cell growth of two lymphoma cell lines, CRL-2261 (non-Hodgkin's lymphoma) and U937 (histiocytic lymphoma), were assayed following treatment with Compound 1 for 36-48 hours. Viability of the cells was determined using a kit from Molecular Probes (Eugene, Oreg.) that labels live cells green (calcein AM) and dead cells red (ethidium homodimer) according to manufacturer's instructions. Fluorescence of calcein and ethidium homodimer in cells was imaged using standard filters for FITC and Texas Red, respectively, with the Nikon TE300 microscope/digital imaging system.

Results

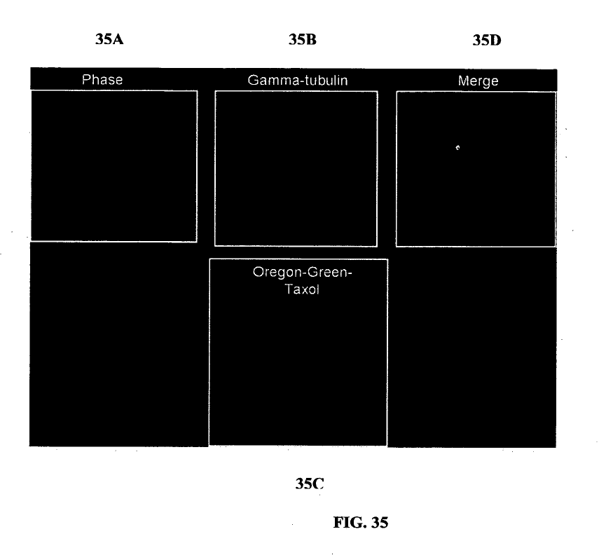

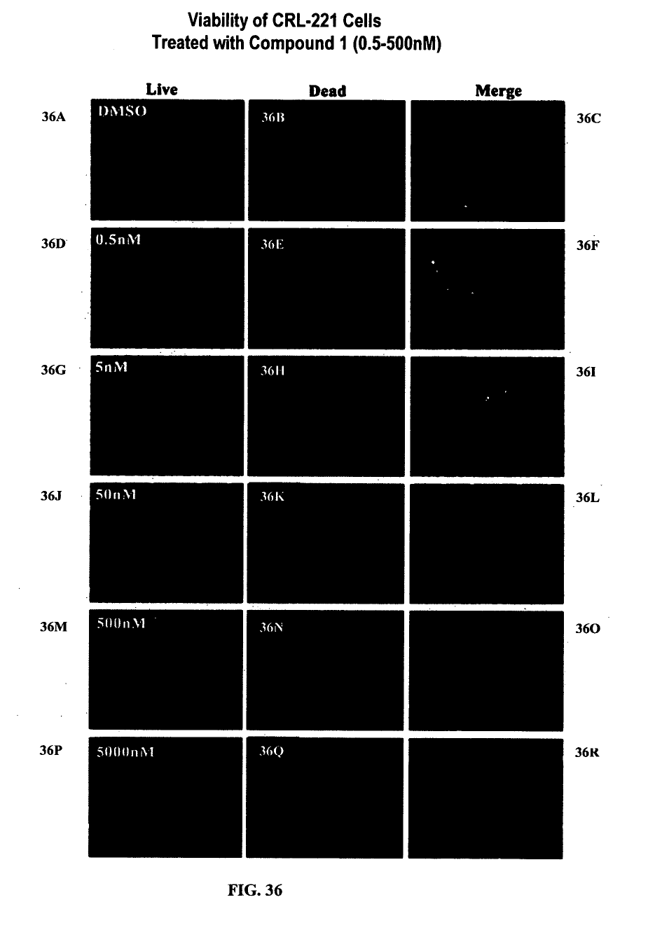

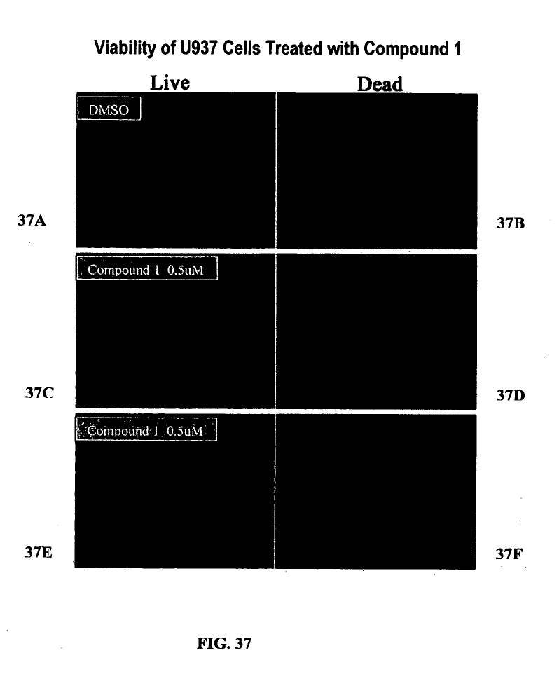

Compound 1 caused significant cell death of CRL-2261 cells (Table 1 and FIG. 36, red cells), while U937 cells were less affected (Table 2 and FIG. 37).

TABLE 1
Compound 1 significantly inhibited CRL-2261
cell growth (48 hour treatment).

| Cell | Treatment | Concentration (nM) | Total cells (×10⁷) |
|---|---|---|---|
| CRL-2261 | DMSO | 1/1000 dilution | 1.19 |
| CRL-2261 | Compound 1 | 0.5 | 1.32 |
| CRL-2261 | Compound 1 | 5 | 1.11 |
| CRL-2261 | Compound 1 | 50 | 0.26 |
| CRL-2261 | Compound 1 | 500 | 0.14 |
| CRL-2261 | Compound 1 | 5000 | 0.16 |

TABLE 2
Compound 1 had a greater effect on CRL-2261
cells than on U937 cells (36 hour treatment).

| Cells | Treatment | Concentration (nM) | Total cells (×10⁶) |
|---|---|---|---|
| U937 | DMSO | 1/1000 dilution | 3.69 |
| U937 | Compound 1 | 500 | 2.13 |
| U937 | Compound 1 | 5000 | 2.40 |
| CRL-2261 | DMSO | 1/1000 dilution | 3.09 |
| CRL-2261 | Compound 1 | 500 | 0.69 |
| CRL-2261 | Compound 1 | 5000 | 0.75 |

Cell counting with flow cytometry also demonstrated that growth of CRL-2261 cells was more significantly inhibited than growth of U937 cells (Table 2). Additional blood cancer cell lines can be tested to further determine the selectivity of Compound 1.

### Example 13

### Isolation of Centrosomes from Compound 1 Treated CHO Cells

Materials and Methods

To study the ultra-structure and proteomic/molecular composition of Compound 1-treated centrosomes, centrosomes were isolated from CHO cells by discontinuous gradient ultracentrifugation using a modification of a procedure described by Ralph Graf (Centrosomes and Spindle Bodies, Methods in Cell Biology, Vol. 67). All chemicals were obtained from Sigma (St. Louis, Mo.) unless indicated otherwise. In brief, cells in the exponential phase of growth were treated with 1 μg/ml of Cytochalasin B and 0.3 μM of Nocodazole for 1.5 hours at 37° C. The cells were then washed in PBS, PBS and 8% (w/v) sucrose, and 8% sucrose, and then lysed in 1 mM Tris (pH 8), 0.1 mM 2-mercaptoethanol, 0.1% Triton X-100. The lysates were cleared with centrifugation at 1500×g for 3 minutes at 4° C. The supernatant was then transferred to Corex tubes, underlaid with 20% Ficoll and centrifuged at 26,000×g for 15 minutes at 4° C. using an HB-4 rotor (Sorvall, Asheville, N.C.). The clear interface was collected and loaded onto a 20-62.5% linear sucrose gradient. Gradients were centrifuged at 70,000×g for 90 minutes at 4° C. using an SW-28 rotor (Beckman Instruments, Fullerton, Calif.). Fractions (0.5 ml) were collected at 4° C. by bottom puncture of centrifuge tubes, and sucrose density was determined with the use of a hand-held refractometer. Fractions between the 48% and 60% (w/w) sucrose concentrations were processed for immunofluorescence.

Results

Gamma-tubulin staining of the centrosome-enriched fraction indicates that centrosomes were successfully isolated (FIG. 35). The exact purity and yield of the procedure is currently under determination. Enriched centrosomes are being subjected to electron microscopy studies to confirm their identity.

While this invention has been particularly shown and described with references to preferred embodiments thereof, it will be understood by those skilled in the art that various changes in form and details may be made therein without departing from the scope of the invention encompassed by the appended claims.

1. A compound, wherein said compound exhibits one or more of the following:
i) disrupts organization of an actin cytoskeleton of a cell;
ii) disrupts organization of a microtubule network of a cell;
iii) induces accumulation of tubulin at centrosomes but does not induce accumulation of tubulin in a nucleus of a cell;
iv) induces accumulation of tubulin at centrosomes at a concentration of 500 nM or less within four hours;
v) induces accumulation of Hsp70 and has weak-to-moderate proteasome inhibitory activity; and
vi) does not have proteasome inhibitory activity when assayed on purified proteasomes;
with the proviso that said compound is not a compound represented by Structural Formula (I): 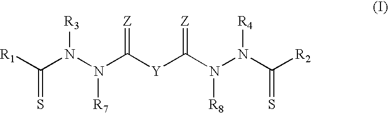
wherein Y is a covalent bond or a substituted or unsubstituted straight chained hydrocarbyl group, or, Y, taken together with both >C═Z groups to which it is bonded, is a substituted or unsubstituted aromatic group;
R₁-R₄ are independently —H, an aliphatic group, a substituted aliphatic group, an aryl group or a substituted aryl group, or R₁ and R₃ taken together with the carbon and nitrogen atoms to which they are bonded, and/or R₂ and R₄ taken together with the carbon and nitrogen atoms to which they are bonded, form a non-aromatic heterocyclic ring optionally fused to an aromatic ring;
R₅ and R₆ are each independently —H, an aliphatic or substituted aliphatic group, or R₅ is —H and R₆ is a substituted or unsubstituted aryl group, or, R₅ and R₆, taken together, are a C2-C6 substituted or unsubstituted alkylene group;
R₇-R₈ are independently —H, an aliphatic group, a substituted aliphatic group, an aryl group or a substituted aryl group; and
Z is ═O or ═S.

2. The compound of claim 1, wherein said compound disrupts organization of an actin cytoskeleton of a cell.

3. The compound of claim 1, wherein said compound disrupts organization of a microtubule network of a cell.

4. The compound of claim 1, wherein said compound induces accumulation of tubulin at centrosomes but does not induce accumulation of tubulin in a nucleus of a cell.

5. The compound of claim 1, wherein said compound induces accumulation of tubulin at centrosomes at a concentration of 500 nM or less within four hours.

6. The compound of claim 1, wherein said compound induces accumulation of Hsp70 and has weak-to-moderate proteasome inhibitory activity.

7. The compound of claim 1, wherein said compound does not have proteasome inhibitory activity when assayed on purified proteasomes.

8-12. (canceled)

13. A method of disrupting centrosome activity in a subject in need thereof comprising administering an effective amount of a compound, wherein said compound exhibits one or more of the following:
i) disrupts organization of an actin cytoskeleton of a cell;
ii) disrupts organization of a microtubule network of a cell;
iii) induces accumulation of tubulin at centrosomes but does not induce accumulation of tubulin in a nucleus of a cell;
iv) induces accumulation of tubulin at centrosomes at a concentration of 500 nM or less within four hours;
v) induces accumulation of Hsp70 and has weak-to-moderate proteasome inhibitory activity; and
vi) does not have proteasome inhibitory activity when assayed on purified proteasomes;
with the proviso that said compound is not a compound represented by Structural Formula (I): 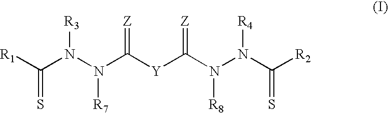
wherein Y is a covalent bond or a substituted or unsubstituted straight chained hydrocarbyl group, or, Y, taken together with both >C═Z groups to which it is bonded, is a substituted or unsubstituted aromatic group;
R₁-R₄ are independently —H, an aliphatic group, a substituted aliphatic group, an aryl group or a substituted aryl group, or R₁ and R₃ taken together with the carbon and nitrogen atoms to which they are bonded, and/or R₂ and R₄ taken together with the carbon and nitrogen atoms to which they are bonded, form a non-aromatic heterocyclic ring optionally fused to an aromatic ring;
R₅ and R₆ are each independently —H, an aliphatic or substituted aliphatic group, or R₅ is —H and R₆ is a substituted or unsubstituted aryl group, or, R₅ and R₆, taken together, are a C2-C6 substituted or unsubstituted alkylene group;
R₇-R₈ are independently —H, an aliphatic group, a substituted aliphatic group, an aryl group or a substituted aryl group; and
Z is ═O or ═S.

14. The method of claim 13, wherein said compound disrupts organization of an actin cytoskeleton of a cell.

15. The method of claim 13, wherein said compound disrupts organization of a microtubule network of a cell.

16. The method of claim 13, wherein said compound induces accumulation of tubulin at centrosomes but does not induce accumulation of tubulin in a nucleus of a cell.

17. The method of claim 13, wherein said compound induces accumulation of tubulin at centrosomes at a concentration of 500 nM or less within four hours.

18. The method of claim 13, wherein said compound induces accumulation of Hsp70 and has weak-to-moderate proteasome inhibitory activity.

19. The method of claim 13, wherein said compound does not have proteasome inhibitory activity when assayed on purified proteasomes.

20-24. (canceled)

25. The method of claim 13, wherein the subject is a human.

26. The method of claim 13, wherein said subject in need thereof has a condition selected from the group consisting of a cancer, a non-cancerous proliferative condition and a Hsp70-responsive disorder.

27. The method of claim 26, wherein said condition is a cancer.

28. A method for treating a condition in a subject comprising administering an effective amount of a compound, wherein said compound exhibits one or more of the following:
i) disrupts organization of an actin cytoskeleton of a cell;
ii) disrupts organization of a microtubule network of a cell;
iii) induces accumulation of tubulin at centrosomes but does not induce accumulation of tubulin in a nucleus of a cell;
iv) induces accumulation of tubulin at centrosomes at a concentration of 500 nM or less within four hours;
v) induces accumulation of Hsp70 and has weak-to-moderate proteasome inhibitory activity; and
vi) does not have proteasome inhibitory activity when assayed on purified proteasomes; and
wherein said condition is selected from the group consisting of fever, muscle disuse (atrophy), denervation, nerve injury, fasting, renal failure associated with acidosis, hepatic failure, uremia, diabetes, sepsis, a closed fracture, an open fracture, a non-union fracture, age-related osteoporosis, post-menopausal osteoporosis, glucocorticoid-induced osteoporosis, disuse osteoporosis, arthritis, periodontal disease and defects, cartilage defects or disorders, male pattern baldness, alopecia caused by chemotherapy, hair thinning resulting from aging, genetic disorders resulting in deficiency of hair coverage, a dry-eye disorder and cystic fibrosis.

29. The method of claim 28, wherein said compound disrupts organization of an actin cytoskeleton of a cell.

30. The method of claim 28, wherein said compound disrupts organization of a microtubule network of a cell.

31. The method of claim 28, wherein said compound induces accumulation of tubulin at centrosomes but does not induce accumulation of tubulin in a nucleus of a cell.

32. The method of claim 28, wherein said compound induces accumulation of tubulin at centrosomes at a concentration of 500 nM or less within four hours.

33. The method of claim 28, wherein said compound induces accumulation of Hsp70 and has weak-to-moderate proteasome inhibitory activity.

34. The method of claim 28, wherein said compound does not have proteasome inhibitory activity when assayed on purified proteasomes.

35. (canceled)

36. The method of claim 28, wherein the subject is a human.

37. The method of claim 28, wherein said compound is a compound represented by Structural Formula (I): 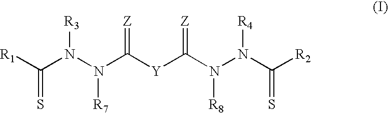
wherein Y is a covalent bond or a substituted or unsubstituted straight chained hydrocarbyl group, or, Y, taken together with both >C═Z groups to which it is bonded, is a substituted or unsubstituted aromatic group;
R₁-R₄ are independently —H, an aliphatic group, a substituted aliphatic group, an aryl group or a substituted aryl group, or R₁ and R₃ taken together with the carbon and nitrogen atoms to which they are bonded, and/or R₂ and R₄ taken together with the carbon and nitrogen atoms to which they are bonded, form a non-aromatic heterocyclic ring optionally fused to an aromatic ring;
R₅ and R₆ are each independently —H, an aliphatic or substituted aliphatic group, or R₅ is —H and R₆ is a substituted or unsubstituted aryl group, or, R₅ and R₆, taken together, are a C2-C6 substituted or unsubstituted alkylene group;
R₇-R₈ are independently —H, an aliphatic group, a substituted aliphatic group, an aryl group or a substituted aryl group; and
Z is ═O or ═S.

38. The method of claim 28, wherein said compound is a compound represented by the following structural formula: 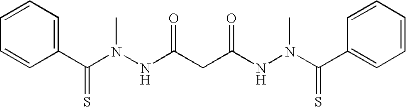
or a pharmaceutically-acceptable salt thereof.

39. The method of claim 37, wherein said compound is a disodium or dipotassium salt.

40. A method of identifying a proteasome inhibitor comprising combining:
a) a cell that expresses tubulin; and
b) a test agent;
and measuring the accumulation of tubulin:
i) at one or more centrosomes of said cell; and/or
ii) in a nucleus of said cell;
wherein an increase in the accumulation of tubulin at said one or more centrosomes and/or said nucleus, relative to a suitable control, indicates that said test agent is a proteasome inhibitor.

41. The method of claim 40, further comprising assaying the test agent using an in vitro and/or an in vivo assay for proteasome inhibitory activity and/or efficacy for treatment of a condition.

42-48. (canceled)

49. A proteasome inhibitor identified by the method of claim 40.

50. A method of identifying a centrosomal proteasome inhibitor comprising combining:
a) a cell that expresses tubulin; and
b) a test agent;
and measuring the accumulation of tubulin:
i) at one or more centrosomes of said cell; and
ii) in a nucleus of said cell;
wherein an increase in the accumulation of tubulin at said one or more centrosomes, but no increase in the accumulation of tubulin at said nucleus, relative to a suitable control, indicates that said test agent is a centrosomal proteasome inhibitor.

51. The method of claim 50, further comprising assaying the test agent using an in vitro and/or an in vivo assay for proteasome inhibitory activity and/or efficacy for treatment of a condition.

52-58. (canceled)

59. A proteasome inhibitor identified by the method of claim 50.

60. A method of identifying a proteasome inhibitor comprising combining:
a) a cell that expresses a centrosome-associated protein; and
b) a test agent;
and measuring the accumulation of said centrosome-associated protein at one or more centrosomes of said cell, wherein an increase in the accumulation of said centrosome-associated protein at said one or more centrosomes, relative to a suitable control, indicates that said test agent is a proteasome inhibitor.

61. The method of claim 60, further comprising assaying the test agent using an in vitro and/or an in vivo assay for proteasome inhibitory activity and/or efficacy for treatment of a condition.

62. The method of claim 60, wherein said centrosome-associated protein is selected from the group consisting of pericentrin, CP140, centrin, alpha-tubulin, beta-tubulin, gamma-tubulin, AKAP450, SKP1p, cyclin-dependent kinase 2-cyclin E (Cdk2-E), kendrin, Protein kinase C-theta, EB1 protein, Nek2, protein kinase A type II isozymes, Hsp70, heat shock Cognate 70 (HSC70), PH33, AIKs, human SCF(SKP2) subunit p19(SKP1), STK15/BTAK, C-Nap1, Tau-like proteins, cyclin E, p53, retinoblastoma protein pRB, BRCA 1, dynein and NuMA.

63-70. (canceled)

71. A proteasome inhibitor identified by the method of claim 60.

72. A method of identifying a nuclear proteasome inhibitor comprising combining:
a) a cell that expresses tubulin; and
b) a test agent;
and measuring the accumulation of tubulin:
i) at one or more centrosomes of said cell; and
ii) in a nucleus of said cell;
wherein an increase in the accumulation of tubulin in the nucleus, but no increase in the accumulation of tubulin at the centrosomes, relative to a suitable control, indicates that said test agent is a nuclear proteasome inhibitor.

73. A method for stabilizing one or more exogenously-expressed protein(s) in a cell comprising contacting a cell with a compound, wherein said compound exhibits one or more of the following:
i) disrupts organization of an actin cytoskeleton of a cell;
ii) disrupts organization of a microtubule network of a cell;
iii) induces accumulation of tubulin at centrosomes but does not induce accumulation of tubulin in a nucleus of a cell;
iv) induces accumulation of tubulin at centrosomes at a concentration of 500 nM or less within four hours;
v) induces accumulation of Hsp70 and has weak-to-moderate proteasome inhibitory activity; and
vi) does not have proteasome inhibitory activity when assayed on purified proteasomes;
with the proviso that said compound is not a compound represented by Structural Formula (I): 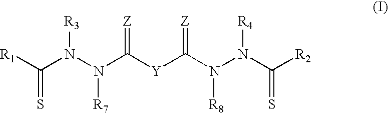
wherein Y is a covalent bond or a substituted or unsubstituted straight chained hydrocarbyl group, or, Y, taken together with both >C═Z groups to which it is bonded, is a substituted or unsubstituted aromatic group;
R₁-R₄ are independently —H, an aliphatic group, a substituted aliphatic group, an aryl group or a substituted aryl group, or R₁ and R₃ taken together with the carbon and nitrogen atoms to which they are bonded, and/or R₂ and R₄ taken together with the carbon and nitrogen atoms to which they are bonded, form a non-aromatic heterocyclic ring optionally fused to an aromatic ring;
R₅ and R₆ are each independently —H, an aliphatic or substituted aliphatic group, or R₅ is —H and R₆ is a substituted or unsubstituted aryl group, or, R₅ and R₆, taken together, are a C2-C6 substituted or unsubstituted alkylene group;
R₇-R₈ are independently —H, an aliphatic group, a substituted aliphatic group, an aryl group or a substituted aryl group; and
Z is ═O or ═S.

74. A method for increasing the efficacy of antigen presentation in a cell comprising contacting the cell with a compound followed by an antigenic peptide, wherein said compound exhibits one or more of the following:
i) disrupts organization of an actin cytoskeleton of a cell;
ii) disrupts organization of a microtubule network of a cell;
iii) induces accumulation of tubulin at centrosomes but does not induce accumulation of tubulin in a nucleus of a cell;
iv) induces accumulation of tubulin at centrosomes at a concentration of 500 nM or less within four hours;
v) induces accumulation of Hsp70 and has weak-to-moderate proteasome inhibitory activity; and
vi) does not have proteasome inhibitory activity when assayed on purified proteasomes;
with the proviso that said compound is not a compound represented by Structural Formula (I): 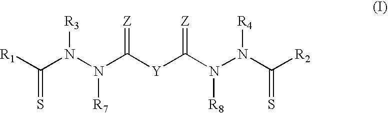
wherein Y is a covalent bond or a substituted or unsubstituted straight chained hydrocarbyl group, or, Y, taken together with both >C═Z groups to which it is bonded, is a substituted or unsubstituted aromatic group;
R₁-R₄ are independently —H, an aliphatic group, a substituted aliphatic group, an aryl group or a substituted aryl group, or R₁ and R₃ taken together with the carbon and nitrogen atoms to which they are bonded, and/or R₂ and R₄ taken together with the carbon and nitrogen atoms to which they are bonded, form a non-aromatic heterocyclic ring optionally fused to an aromatic ring;
R₅ and R₆ are each independently —H, an aliphatic or substituted aliphatic group, or R₅ is —H and R₆ is a substituted or unsubstituted aryl group, or, R₅ and R₆, taken together, are a C2-C6 substituted or unsubstituted alkylene group;
R₇-R₈ are independently —H, an aliphatic group, a substituted aliphatic group, an aryl group or a substituted aryl group; and
Z is ═O or ═S.

75. A method for identifying a compound that disrupts centrosome activity comprising combining:
a) a cell that expresses a centrosome-associated protein; and
b) a test agent;
and measuring the accumulation of the centrosome-associated protein:
i) at one or more centrosomes of the cell; and
ii) in a nucleus of the cell;
wherein an increase in the accumulation of the centrosome-associated protein at the one or more centrosomes, but no increase in the accumulation of the centrosome-associated protein at the nucleus, relative to a suitable control, indicates that said test agent is a compound that disrupts centrosome activity.

76. The method of claim 75, wherein said centrosome-associated protein is selected from the group consisting of pericentrin, CP 140, centrin, alpha-tubulin, beta-tubulin, gamma-tubulin, AKAP450, SKP1p, cyclin-dependent kinase 2-cyclin E (Cdk2-E), kendrin, Protein kinase C-theta, EB1 protein, Nek2, protein kinase A type II isozymes, Hsp70, heat shock Cognate 70 (HSC70), PH33, AIKs, human SCF(SKP2) subunit p19(SKP1), STK15/BTAK, C-Nap1, Tau-like proteins, cyclin E, p53, retinoblastoma protein pRB, BRCA1, dynein and NuMA.
